(19) United States
(12) Patent Application Publication
(10) Pub. No.: US 2011/0189305 A1
(43) Pub. Date: Aug. 4, 2011

(54) TREATMENT OF LUNG CANCER

(76) Inventor: Cynthia W. Tuthill, Menlo Park, CA (US)

(21) Appl. No.: 12/674,646

(22) PCT Filed: Aug. 21, 2008
(86) PCT No.: PCT/US08/09932

§ 371 Date: Sep. 22, 2010

Related U.S. Application Data
(60) Provisional application No. 60/957,530, filed on Aug. 23, 2007.

(51) Int. Cl. A61K 33/24; A61K 31/405; A61K 31/513; A61K 31/655; A61P 35/00

(52) U.S. Cl. 424/649; 514/419; 514/274; 514/151

(57) ABSTRACT

An immunomodulatory compound is administered to treat, prevent, inhibit, or reduce lung cancer in a subject.

The present application claims the benefit of U.S. Provisional Application Ser. No. 60/957,530, filed Aug. 23, 2007, the disclosures of which is incorporated herein in its entirety by reference.

### BACKGROUND OF THE INVENTION

1. Field of the Invention

The present invention relates to the field of treatment of lung cancer.

2. Description of Background Art

Lung cancer is the malignant transformation and expansion of lung tissue, and is responsible for 1.3 million deaths worldwide annually. It is the most common cause of cancer-related death in men, and the second most common in women.

Current research indicates that the factor with the greatest impact on risk of lung cancer is long-term exposure to inhaled carcinogens, especially tobacco smoke. The occurrence of lung cancer in others (less than one tenth) appears to be due to a combination of genetic factors. Radon gas and air pollution may also contribute to the development of lung cancer.

Treatment and prognosis depend upon the histological type of cancer, the stage (degree of spread), and the patient's performance status. Current treatments include surgery, chemotherapy, and radiotherapy. Overall, the five-year survival rate is about 14%.

There are two main types of lung cancer categorized by the size and appearance of the malignant cells seen by a histopathologist under a microscope: non-small cell (80%) and small-cell (roughly 20%) lung cancer. This classification, although based on simple histological criteria, has very important implications for clinical management and prognosis of the disease.

The non-small cell lung cancers (NSCLC) are grouped together because their prognosis and management are roughly identical. There are three main sub-types: squamous cell lung carcinoma, adenocarcinoma and large cell lung carcinoma.

Squamous cell carcinoma, accounting for 29% of lung cancers, also starts in the larger bronchi but grows slower. The size of these tumours varies on diagnosis.

Adenocarcinoma is the most common subtype of NSCLC, accounting for 32% of lung cancers. It is a form which starts near the gas-exchanging surface of the lung. Most cases of adenocarcinoma are associated with smoking. However, among people who have never smoked (“never-smokers”), adenocarcinoma is the most common form of lung cancer. A subtype of adenocarcinoma, the bronchioloalveolar carcinoma, is more common in female never-smokers, and may have different responses to treatment.

Large cell carcinoma is a fast-growing form, accounting for 9% of lung cancers, that grows near the surface of the lung.

Small cell lung cancer (SCLC, also called “oat cell carcinoma”) is a less common form of lung cancer. It tends to start in the larger breathing tubes and grows rapidly becoming quite large. The oncogene most commonly involved is L-myc. The “oat” cell contains dense neurosecretory granules which give this an endocrine/paraneoplastic syndrome association. It is initially more sensitive to chemotherapy, but ultimately carries a worse prognosis and is often metastatic at presentation. This type of lung cancer is strongly associated with smoking.

Other types of lung cancers include carcinoid, adenoid cystic carcinoma (cylindroma) and mucoepidermoid carcinoma.

### Metastatic Cancers

The lung is a common place for metastasis from tumors in other parts of the body. The adrenal glands, liver, brain, and bone are the most common sites of metastasis from primary lung cancer itself.

There remains a need in the art for methods of treatment for treating, preventing, inhibiting or reducing lung cancer.

### SUMMARY OF THE INVENTION

In accordance with the present invention, a method of treatment for treating, inhibiting, reducing or at least partly preventing lung cancer, a metastasis thereof, or a metastasis in a lung from a cancer outside the lung, or for treating, inhibiting, reducing or at least partly preventing growth of lung cancer cells, a metastasis thereof, or a metastasis of cancer cells in a lung from cancer cells outside the lung, in a subject, comprises administering to the subject a treatment-effective amount of an immunomodulator compound of formula A

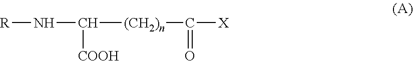

wherein, n is 1 or 2, R is hydrogen, acyl, alkyl or a peptide fragment, and X is an aromatic or heterocyclic amino acid or a derivative thereof, so as to treat, inhibit, reduce or at least partly prevent said lung cancer, a metastasis thereof, or a metastasis in a lung from a cancer outside the lung in the subject, or treat, inhibit, reduce or at least partly prevent growth of said lung cancer cells, a metastasis thereof, or a metastasis of cancer cells in a lung from cancer cells outside the lung, in the subject.

### BRIEF DESCRIPTION OF THE DRAWINGS

FIG. 1 graphically depicts tumor growth in one study of one embodiment at different dosages.

FIG. 2 graphically depicts tumor weight in the study of one embodiment at different dosages.

### DESCRIPTION OF THE PREFERRED EMBODIMENTS

In accordance with one embodiment, the present invention relates to a method of treatment for treating, at least partly preventing, inhibiting, or reducing lung cancer by administering an immunomodulator compound to a mammalian subject, preferably a human patient.

In certain embodiments, the disease is lung cancer, a metastasis thereof, or a metastasis in a lung from a cancer outside the lung. The invention can be utilized to treat, at least partly prevent, inhibit or reduce growth of lung cancer cells, a metastasis thereof, or a metastasis of cancer cells in a lung from cancer cells outside the lung, in a subject. In some embodiments, the primary lung cancer tumor, or a major portion thereof, is removed by surgery before, during or after treatment with a compound of the invention.

Immunomodulator compounds in accordance with the present invention comprise immunomodulators of Formula A:

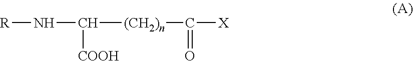

In Formula A, n is 1 or 2, R is hydrogen, acyl, alkyl or a peptide fragment, and X is an aromatic or heterocyclic amino acid or a derivative thereof. Preferably, X is L-tryptophan or D-tryptophan, most preferably L-tryptophan.

Appropriate derivatives of the aromatic or heterocyclic amino acids for “X” are: amides, mono- or di-(C₁-C₆) alklyl substituted amides, arylamides, and (C₁-C₆) alkyl or aryl esters. Appropriate acyl or alkyl moieties for “R” are: branched or unbranched alkyl groups of 1 to about 6 carbons, acyl groups from 2 to about 10 carbon atoms, and blocking groups such as carbobenzyloxy and t-butyloxycarbonyl. Preferably the carbon of the CH group shown in Formula A has a stereoconfiguration, when n is 2, that is different from the stereoconfiguration of X.

Preferred embodiments utilize compounds such as γ-D-glutamyl-L-tryptophan, γ-L-glutamyl-L-tryptophan, γ-L-glutamyl-Nᵢₙ-formyl-L-tryptophan, N-methyl-γ-L-glutamyl-L-tryptophan, N-acetyl-γ-L-glutamyl-L-tryptophan, γ-L-glutamyl-D-tryptophan, β-L-aspartyl-L-tryptophan, and β-D-aspartyl-L-tryptophan. Particularly preferred embodiments utilize γ-D-glutamyl-L-tryptophan, sometimes referred to as SCV-07. These compounds, methods for preparing these compounds, pharmaceutically acceptable salts of these compounds and pharmaceutical formulations thereof are disclosed in U.S. Pat. No. 5,916,878, incorporated herein by reference.

SCV-07, γ-D-glutamyl-L-tryptophan, is a member of a class of immunomodulatory drugs that possess γ-glutamyl or β-aspartyl moieties, which was discovered by Russian scientists and is being examined for efficacy in several indications in the U.S. by SciClone Pharmaceuticals, Inc. SCV-07 possesses a number of immunomodulatory activities in vivo and in vitro. SCV-07 increases Con-A-induced thymocyte and lymphocyte proliferation, increases Con-A-induced interleukin-2 (IL-2) production and IL-2 receptor expression by spleen lymphocytes, and stimulates expression of Thy-1.2 on bone marrow cells. In vivo, SCV-07 has a strong immunostimulatory effect on 5-FU-immune-suppressed animals and in a model of immunization with sheep red blood cells.

The Formula A compounds may be administered at any effective dosage, e.g., at dosages in the range of about 0.001-1000 mg, preferably about 0.1-100 mg and most preferably about 10 mg. Dosages may be administered one or more times per week, e.g., on a daily basis, with dosages administered one or more times per day. Administration can be by any suitable method, including orally, nasally, transdermally, sublingually, by injection, periodic infusion, continuous infusion, and the like. The dosages may be administered by intramuscular injection, although other forms of injection and infusion may be utilized, and other forms of administration such as oral or nasal inhalation or oral ingestion may be employed. Aerosols, solutions, suspensions, dispersions, tablets, capsules, syrups, etc., may be utilized.

Dosages may also be measured in milligrams per kilogram, with dosages in the range of about 0.00001-1000 mg/kg, more preferably within the range of about 0.01-100 mg/kg, still more preferably about 0.1-50 mg/kg, and still more preferably about 1-20 mg/kg.

Included are biologically active analogs having substituted, deleted, elongated, replaced, or otherwise modified portions which possess bioactivity substantially similar to that of SCV-07, e.g., an SCV-07 derived peptide having sufficient homology with SVC-07 such that it functions in substantially the same way with substantially the same activity as SCV-07.

According to one embodiment, a Formula A compound may be administered to a subject so as to substantially continuously maintain an effective amount of the Formula A compound in the subject's circulatory system during a treatment or prevention period. Although much longer treatment periods are contemplated in accordance with the present invention, embodiments of the invention include substantially continuously maintaining an effective amount of the Formula A compound in the patient's circulatory system during treatment periods of at least about 6, 10, 12 hours, or longer. In other embodiments, treatment periods are for at least about a day, and even for a plurality of days, e.g., a week or longer. However, it is contemplated that treatments, as defined above, in which effective amounts of the Formula A compound are substantially continuously maintained in the subject's circulatory system, may be separated by non-treatment periods of similar or different durations.

In accordance with one embodiment, the Formula A compound is continuously infused into a subject, e.g., by intravenous infusion, during the treatment period, so as to substantially continuously maintain an effective amount of the Formula A compound in the subject's circulatory system. The infusion may be carried out by any suitable means, such as by minipump. Alternatively, an injection regimen of the Formula A compound can be maintained so as to substantially continuously maintain an effective amount of the Formula A compound in the subject's circulatory system. Suitable injection regimens may include an injection every 1, 2, 4, 6, etc. hours, so as to substantially continuously maintain the effective amount of the Immunomodulator compound peptide in the subject's circulatory system during the treatment period.

Although it is contemplated that during continuous infusion of the Formula A compound, administration will be for a substantially longer duration, according to one embodiment the continuous infusion of the Formula A compound is for a treatment period of at least about 1 hour. More preferably, continuous infusion is carried out for longer periods, such as for periods of at least about 6, 8, 10, 12 hours, or longer. In other embodiments, continuous infusion is for at least about one day, and even for a plurality of days such as for one week or more.

In some embodiments, the Formula A compound is present in a pharmaceutically acceptable liquid carrier, such as water for injection, physiological saline, or similar, at concentrations within a range of about 0.001-1000 μg/ml, more preferably about 0.1-100 μg/ml.

Effective amounts of Formula A compound can be determined by routine dose-titration experiments.

The Formula A compound also can be administered with other agents. For example, with treatments of cancer such agents include chemotherapy agents and/or radiation.

Radiation may be administered by any suitable method, and at any suitable dosage and dosage regimen administered in the art. For example, radiation can be administered at a dosage rate of approximately 1 Gy/minute, and radiation can be administered, for example, at two doses per day of, e.g., about 4 Gy/dose on separate days of administration, separated by a day of non administration of radiation.

Chemotherapy agents that may be administered in a treatment regimen along with the Formula A compounds include any suitable chemotherapy agent, such as, without limitation, cisplatin, 5-fluorouracil (5-Fu), DTIC, and/or the like. Such chemotherapy agents may be administered at any suitable dosage and/or dosage regimen, including those set forth in the examples herein.

### Example 1

### Abbreviations

### CTX Cyclophosphamide

### F Female

### g Gram

### IR Inhibition Rate

### Ip Intraperitoneal

### Kg Kilogram

### L Length

### M Male

### mL Milliliter

### sc Subcutaneous

### SD Standard Deviation

### W Width

### Summary

In this study, SCV-07 was tested for its inhibitory effect on growth of murine lung tumor in C57/BL6 mice. A total of 70 mice were implanted subcutaneously with murine Lewis lung cancer (LLC) cells, followed by treatment with SCV-07 or cyclophosphamide (CTX) alone or in combination for 14 consecutive days. SCV-07 was administered daily by sc injection, while CTX was administered by ip injection every other day. In total, 7 groups were used: Group 1: vehicle; Group 2: CTX 20 mg/kg; Group 3: CTX 40 mg/kg; Group 4: SCV-07 5 mg/kg; Group 5: SCV-07 10 mg/kg; Group 6: SCV-07 5 mg/kg plus CTX 20 mg/kg; Group 7: SCV-07 10 mg/kg plus CTX 20 mg/kg. Tumor volume and body weight were measured every three days, and tumor weights were measured on Day 16 (necropsy day) at the end of the study.

Throughout the course of the study, animal death was not found in any group. Moreover, the statistical results of body weights showed no significant differences between SCV-07 alone groups and vehicle control group, indicating no effect of SCV-07 on animal growth. By contrast, from Day 6 onwards, CTX treatment groups showed a significant decrease in body weight, especially in the groups administered with a high dose of CTX.

For the tumor growth, on day 3, all of groups except Group 4 showed statistically significant inhibition of tumor volume compared to Group 1 (vehicle control). On Day 6, only Group 2 and Group 3 showed the inhibition. On Day 9, Group 2, Group 3 and Group 7 showed the inhibition. On Day 12, all groups except Group 4 showed the inhibition. On Day 15, the mean tumor sizes of all groups were statistically significantly smaller than Group 1. On Day 16, the mean tumor weights of all treatment groups were lower than the vehicle control group. The tumor weight inhibition rate of Group 2, Group 3, Group 4, Group 5, Group 6 and Group 7 were 45.54% (p<0.01), 90.25% (p<0.01), 18.08% (p=0.07), 30.60% (p<0.01), 48.57% (p<0.01) and 62.63% (p<0.01), respectively.

In conclusion, the tumor model used in this study is valid because vehicle group showed significant tumor growth, while the positive control drug CTX effectively reduced the tumor growth. Daily administration of SCV-07 (10 mg/kg) for 14 days significantly inhibited the tumor growth. Tumor weights in treated animals were significantly reduced in comparison with those of in the vehicle control group. Moreover, the combined treatment of CTX at the suboptimal dose (20 mg/kg) with high dose of SCV-07 (10 mg/kg) showed increased anti-tumor efficacy.

### Introduction

This study tests the anti-tumor effect of SCV-07 with murine lung cancer model to explore its potential as an anti-tumor drug in lung cancer. CTX is used as positive control. The combination effects of SCV-07 and CTX is also tested to determine if there exists additive or synergistic effect.

### Materials and Methods

### Test and Control Articles

PBS was used as the negative control article (vehicle), and CTX as the positive control. CTX was purchased from Sigma-Aldrich and aliquoted to 10 mg/vial. PBS was added to achieve the proper dose level as indicated in the study design table. The formulation was kept on ice, protected from light, and used immediately. Test article (SCV-07) is dissolved in PBS to achieve the proper dose levels as indicated on the study design table; kept on ice, protected from light, and used within one week.

### Test System and Animal Husbandry

### Murine Lung Cancer Cells (LLC)

Murine Lewis lung cancer cells were obtained from the Cell Culture Center of Chinese Academy of Medical Sciences (CAMS; Beijing, P. R. China). The cancer cells were adapted in C57BL/6 mice before use in experiment. Please refer to Section 4.3.1 for details on cell adaptation.

### Test System

Thirty-five male and thirty-five female healthy, naive, C57BL/6 mice were received from the Institute of Laboratory Animal Science, CAMS, Beijing, P. R. China. The animals were six weeks old and weighed between 18 and 22 grams at the start of the study.

### Animal Husbandry

Animals were group-housed in autoclaved shoe box cages with autoclaved wood chips as the bedding materials. The temperature of the animal room was maintained at 22 to 25° C., and the relative humidity was maintained at 40 to 60%. A 12-hour light/12-hour dark cycle was maintained except when interrupted by study-related events. Animals were fed ad libitum with sterile water and Beijing KeAoXieLi Rodent Diet (certified). All animals were acclimated for 3 days before tumor inoculation.

### Experimental Procedures

### Tumor Cell Adaptation

As per aseptic tissue culture procedures, one vial of murine lung cancer cells was thawed and centrifuged at 1000 rpm, 20-25° C. for 5 minutes. The cell pellets were suspended in 0.5 mL normal saline (NS) and subcutaneously injected into the right axilla of each mouse (approximately 1×10⁶ cells/mouse). When the tumor diameter reached approximately 1 cm, the animals were euthanized with CO₂ asphyxiation and the tumor was excised. Tumor cells were suspended in normal saline as previously described and the cell adaptation cycle was repeated one more time.

### Tumor Cell Inoculation

1×10⁶ LLC cells in a volume of 0.1 mL of normal saline were subcutaneously injected into the right axillary area of the mouse. The day of tumor inoculation was defined as Day 0.

### Study Design and Treatment Regimen

On Day 1, the animals were randomized into seven different groups based on their body weights so that the mean body weights were not statistically significantly different among groups. Dosing was initiated on Day 1. SCV-07 was administered once daily for 14 consecutive days via subcutaneous (sc) administration in a dose volume of 0.1 mL/20 g body weight, and CTX was administered by intraperitoneal injection every other day at the same dose volume. The vehicle was also dosed once daily for 14 consecutive days via sc administration of PBS at the same dose volume. Treatment regimens for all groups are outlined in Table 1.

TABLE 1
Treatment Regimen and Study Design

| Group | | | Number of | Dosing | Necropsy |
|---|---|---|---|---|---|
| Number | Group Name | Treatment | Animals | Days | Day |
| 1 | Vehicle Control | PBS | 10 | 1 through | Day 16 |
| 2 | Positive Control 1 | CTX 20 mg/kg, i.p., | 10 | 14 | |
| | | every other day | | | |
| 3 | Positive Control 2 | CTX 40 mg/kg, i.p., | 10 | | |
| | | every other day | | | |
| 4 | Test Article 1 | SCV-07, 5 mg/kg, | 10 | | |
| | | s.c., daily | | | |
| 5 | Test Article 2 | SCV-07, 10 mg/kg, | 10 | | |
| | | s.c., daily | | | |
| 6 | Combination 1 | SCV-07, 5 mg/kg, | 10 | | |
| | | s.c., daily + | | | |
| | | CTX 20 mg/kg, i.p., | | | |
| | | every other day | | | |
| 7 | Combination 2 | SCV-07, 10 mg/kg, | 10 | | |
| | | s.c., daily + | | | |
| | | CTX 20 mg/kg, i.p., | | | |
| | | every other day | | | |

### Evaluation of Anti-Tumor Effect

Throughout the course of the study, the tumor sizes and body weights of all animals were measured every 3 days—tumor size measured by caliper and body weight by laboratory balance. Animal mortality and morbidity were daily monitored and recorded. On Day 16, the animals were euthanized by CO₂ asphyxiation, and the tumors were excised, separated, and weighed.

Tumor volume was calculated using the following formula:

Tumor Volume=Length×Width×Width/2

Tumor volume inhibition rate (IR) was calculated according to the formula below:

IR(TV)=(TV_{vehicle}−TV_{drug treated})/TV_{vehicle}×100%

Where, TV is the tumor volume on the day of measurement, “vehicle” denotes the group receiving PBS, and “drug treated” denotes groups receiving SCV-07 and/or CTX.

The anti-tumor effect of SCV-07 used alone or in combination with CTX was also evaluated by tumor weight. The tumor weight of each mouse was recorded after euthanasia, and the inhibition rate of tumor weight was calculated according to the formula below:

IR(TW)=(Tumor weight_{vehicle}−Tumor weight_{drug treated})/Tumor weight_{vehicle}×100%

Mean and standard deviations were calculated using Excel.

### Statistical Analysis

Inter-group comparison was performed in terms of tumor volume, tumor weight and body weight, using a student's t test. P values of less than 0.05 were considered to be statistically significant.

### Results and Discussion

### Mortality

No animal deaths were observed in the study.

### Tumor Size

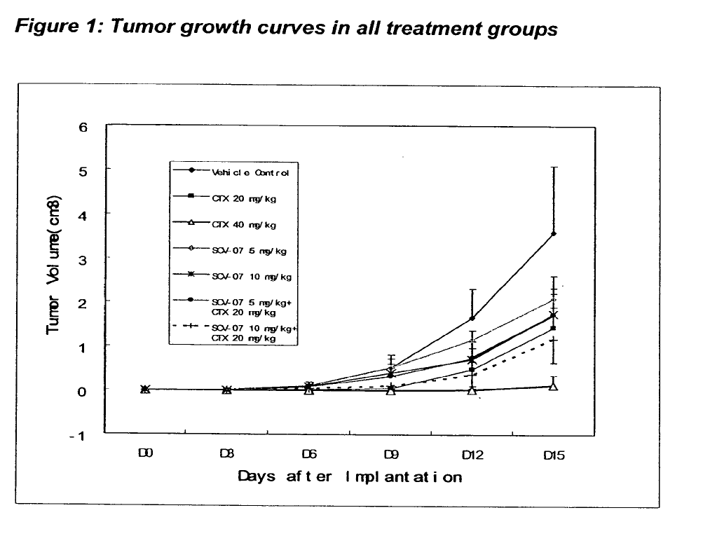

Raw measurement data of tumor size are listed in Appendixes 1-10. The calculated tumor inhibition rates and statistical comparison of each treatment group versus vehicle group are listed in the Tables 2-6. The tumor growth curves are illustrated in FIG. 1. Based on tumor volume data, all groups except Group 4 showed significant inhibition of tumor growth on Day 3. Group 2 and Group 3 showed inhibition on Day 6. On Day 9, the mean tumor sizes of Group 2, Group 3 and Group 7 were statistically significantly smaller than Group 1 (vehicle). On Day 12, the mean tumor sizes of all treatment groups except Group 4 were statistically significantly smaller than Group 1. On Day 15, the mean tumor sizes of all groups were statistically significantly smaller than Group 1. Among combination treatment groups, high dose of SCV-07 showed additive effect with CTX.

### Tumor Weight

Raw data of tumor weight were shown in Appendix 11, while statistical comparison results between each of treatment groups and vehicle control group are tabulated in Table 7. As shown in Table 7, the mean tumor weights measured on Day 16 of all treatment groups were lower than the vehicle control group. The tumor inhibition rates in Group 2, Group 3, Group 4, Group 5, Group 6, and Group 7 were 45.54% (p<0.01), 90.25% (p<0.01), 18.08% (p=0.07), 30.60% (p<0.01), 48.57% (p<0.01), and 62.63% (p<0.01), respectively. There was no statistically significant difference in tumor inhibition between Group 2 (CTX 20 mg/kg) and Group 6 (CTX 20 mg/kg+SCV-07 5 mg/kg). By contrast, the inhibition rate in Group 7 (CTX 20 mg/kg+SCV-07 10 mg/kg) was statistically significantly greater than in Group 2 (CTX 20 mg/kg), indicating an additive effect when using CTX in combination with SCV-07.

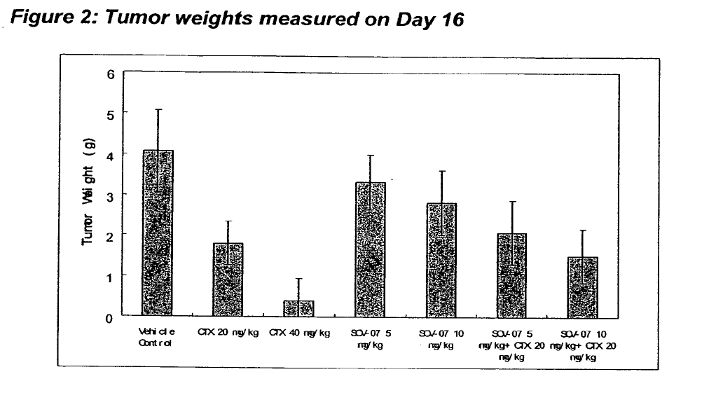

FIG. 2 illustrates the tumor weight for all groups at the end of the study (Day 16).

### Body Weight

Raw data of body weight measurement were listed in Appendixes 12-17. The results of statistical comparison of each treatment group versus vehicle group are listed in the Tables 8-13.

As shown in the tables, there are no significant differences between each of treatment groups and vehicle control group on Day 3. On Day 6, Group 3 (CTX 40 mg/kg) exhibited a body weight inhibition rate of 10.82% (P<0.05). There was no significant difference of body weights for other groups. On Day 9, the inhibition rates for group 2 (CTX 20 mg/kg), group 3 (CTX 40 mg/kg) and group 6 (CTX 20 mg/kg+SCV-07 5 mg/kg) were 12.35% (p<0.01), 16.12% (p<0.01) and 7.22% (p<0.01), respectively. With the addition of SCV-07 in Group 6, the inhibition rate of body weight gain was decreased. There was no significant difference of body weights for other groups, indicating that SCV-07 had no effect on animal body weight gain. On Day 12 the inhibition rates for Group 2 (CTX 20 mg/kg), Group 3 (CTX 40 mg/kg), and Group 6 (CTX 20 mg/kg+SCV-07 5 mg/kg) were 14.83% (p<0.01), 21.97% (p<0.01), and 10.28% (p<0.01), respectively. On Day 15, the inhibition rate in Group 3 (CTX 40 mg/kg) was 25% (p<0.01). There was no significant difference of body weights for other groups. This result indicated that CTX inhibited the body weight gain probably due to its toxicity and SCV-07 could partially reserve the inhibition, probably due to its alleviation of CTX toxicity.

### Conclusion and Discussion

In conclusion, the tumor model used in this study is valid as tumor growth can be inhibited by positive control drug CTX. Daily administration of test article, SCV-07 at 10 mg/kg for 14 days is also effective against the tumor growth. Tumor sizes in animals of all SCV-07-treated groups were significantly reduced in comparison with those of the vehicle control group from Day 12 onwards. Tumor weights, which are measured on Day 16, are also significantly reduced in the group receiving 10 mg/kg SCV-07 alone and in the groups receiving combination therapy, but not in the group receiving 5 mg/kg SCV-07 alone. Furthermore, the combined use of 10 mg/kg SCV-07 and 20 mg/kg SCV-07 collectively produced 62.63% inhibition of tumor growth, in comparison to 30.6% and 45.54% inhibition obtained when using 10 mg/kg SCV-07 or 20 mg/kg CTX alone. These results suggest that combined use of SCV-07 with CTX produces an additive effect towards the tumor growth inhibition.

The mean animal body weights in CTX treatment groups were significantly decreased, indicating a toxic effect. However, with the addition of SCV-07 in the combination therapy groups, it appears that the toxic effect of CTX can be attenuated at least partially by SCV-07. The phenomenon is apparent on Day 9, when CTX 20 mg/kg alone produces statistically significant inhibition of body weight gain, but the inhibition is abolished in the combination treatment group (CTX 20 mg/kg+SCV-07 10 mg/kg). We did not test protective effect of SCV-07 in combination with higher dose of CTX (i.e., 40 mg/kg) that produced more pronounced inhibition of body weight gain. The protective effect of SCV-07, if verified by future studies, shall be very useful when considering SCV-07 and CTX combination therapy. Taken together, combination of suboptimal dose of CTX (20 mg/kg) with high dose of SCV-07 (10 mg/kg) showed increased anti-tumor efficacy and less toxicity.

### Tables

TABLE 2
Statistical results of tumor sizes on Day 3

| | | | Number of | Average | | |
|---|---|---|---|---|---|---|
| Group | | | Animals | Weight | IR | P |
| Number | Group Name | Treatment | Surviving | (x ± SD) | (TV) % | value |
| 1 | Vehicle | PBS | 10 | 0.0172 ± 0.0078 | | |
| | Control | | | | | |
| 2 | Positive | CTX 20 mg/kg, i.p., every | 10 | 0.0022 ± 0.0043 | 87.21 | 0.0000 |
| | Control 1 | other day | | | | |
| 3 | Positive | CTX 40 mg/kg, i.p., every | 10 | 0.0014 ± 0.0043 | 91.86 | 0.0000 |
| | Control 2 | other day | | | | |
| 4 | Test Article 1 | SCV-07, 5 mg/kg, s.c., | 10 | 0.0173 ± 0.0137 | −0.29 | 0.9921 |
| | | daily | | | | |
| 5 | Test Article 2 | SCV-07, 10 mg/kg, s.c., | 10 | 0.0072 ± 0.0068 | 58.43 | 0.0066 |
| | | daily | | | | |
| 6 | Combination 1 | SCV-07, 5 mg/kg, s.c., | 10 | 0.0094 ± 0.0100 | 45.35 | 0.0677 |
| | | daily + CTX 20 mg/kg, | | | | |
| | | i.p., every other day | | | | |
| 7 | Combination 2 | SCV-07, 10 mg/kg, s.c., | 10 | 0.0070 ± 0.0058 | 59.30 | 0.0038 |
| | | daily + CTX 20 mg/kg, | | | | |
| | | i.p., every other day | | | | |

TABLE 3
Statistical results of tumor sizes on Day 6

| | | | Number of | Average | | |
|---|---|---|---|---|---|---|
| Group | | | Animals | Weight | IR | P |
| Number | Group Name | Treatment | Surviving | (x ± SD) | (TV) % | value |
| 1 | Vehicle | PBS | 10 | 0.1091 ± 0.0857 | | |
| | Control | | | | | |
| 2 | Positive | CTX 20 mg/kg, i.p., every | 10 | 0.0217 ± 0.0248 | 80.15 | 0.0062 |
| | Control 1 | other day | | | | |
| 3 | Positive | CTX 40 mg/kg, i.p., every | 10 | 0.0067 ± 0.0196 | 93.86 | 0.0017 |
| | Control 2 | other day | | | | |
| 4 | Test Article 1 | SCV-07, 5 mg/kg, s.c., | 10 | 0.0908 ± 0.0224 | 16.78 | 0.5216 |
| | | daily | | | | |
| 5 | Test Article 2 | SCV-07, 10 mg/kg, s.c., | 10 | 0.0871 ± 0.0366 | 20.17 | 0.4648 |
| | | daily | | | | |
| 6 | Combination 1 | SCV-07, 5 mg/kg, s.c., | 10 | 0.0824 ± 0.0560 | 24.44 | 0.4209 |
| | | daily + CTX 20 mg/kg, | | | | |
| | | i.p., every other day | | | | |
| 7 | Combination 2 | SCV-07, 10 mg/kg, s.c., | 10 | 0.0601 ± 0.0406 | 44.89 | 0.1199 |
| | | daily + CTX 20 mg/kg, | | | | |
| | | i.p., every other day | | | | |

TABLE 4
Statistical results of tumor sizes on Day 9

| | | | Number of | Average | | |
|---|---|---|---|---|---|---|
| Group | | | Animals | Weight | IR | P |
| Number | Group Name | Treatment | Surviving | (x ± SD) | (TV) % | value |
| 1 | Vehicle | PBS | 10 | 0.5252 ± 0.2953 | | |
| | Control | | | | | |
| 2 | Positive | CTX 20 mg/kg, i.p., every | 10 | 0.0582 ± 0.0100 | 88.92 | 0.0001 |
| | Control 1 | other day | | | | |
| 3 | Positive | CTX 40 mg/kg, i.p., every | 10 | 0.0085 ± 0.0195 | 98.39 | 0.0000 |
| | Control 2 | other day | | | | |
| 4 | Test Article 1 | SCV-07, 5 mg/kg, s.c., | 10 | 0.5286 ± 0.1945 | −0.66 | 0.9757 |
| | | daily | | | | |
| 5 | Test Article 2 | SCV-07, 10 mg/kg, s.c., | 10 | 0.4001 ± 0.2034 | 23.82 | 0.2845 |
| | | daily | | | | |
| 6 | Combination 1 | SCV-07, 5 mg/kg, s.c., | 10 | 0.3324 ± 0.1944 | 36.71 | 0.1018 |
| | | daily + CTX 20 mg/kg, | | | | |
| | | i.p., every other day | | | | |
| 7 | Combination 2 | SCV-07, 10 mg/kg, s.c., | 10 | 0.0998 ± 0.0602 | 81.01 | 0.0003 |
| | | daily + CTX 20 mg/kg, | | | | |
| | | i.p., every other day | | | | |

TABLE 5
Statistical results of tumor sizes on Day 12

| | | | Number of | Average | | |
|---|---|---|---|---|---|---|
| Group | | | Animals | Weight | IR | P |
| Number | Group Name | Treatment | Surviving | (x ± SD) | (TV) % | value |
| 1 | Vehicle | PBS | 10 | 1.6607 ± 0.6599 | | |
| | Control | | | | | |
| 2 | Positive | CTX 20 mg/kg, i.p., every | 10 | 0.4864 ± 0.1807 | 70.71 | 0.0000 |
| | Control 1 | other day | | | | |
| 3 | Positive | CTX 40 mg/kg, i.p., every | 10 | 0.0149 ± 0.0333 | 99.11 | 0.0000 |
| | Control 2 | other day | | | | |
| 4 | Test Article 1 | SCV-07, 5 mg/kg, s.c., | 10 | 1.1649 ± 0.2079 | 29.85 | 0.0360 |
| | | daily | | | | |
| 5 | Test Article 2 | SCV-07, 10 mg/kg, s.c., | 10 | 0.7196 ± 0.2452 | 56.67 | 0.0005 |
| | | daily | | | | |
| 6 | Combination 1 | SCV-07, 5 mg/kg, s.c., | 10 | 0.7505 ± 0.3700 | 54.81 | 0.0013 |
| | | daily + CTX 20 mg/kg, | | | | |
| | | i.p., every other day | | | | |
| 7 | Combination 2 | SCV-07, 10 mg/kg, s.c., | 10 | 0.3573 ± 0.2481 | 78.49 | 0.0000 |
| | | daily + CTX 20 mg/kg, | | | | |
| | | i.p., every other day | | | | |

TABLE 6
Statistical results of tumor sizes on Day 15

| | | | Number of | Average | | |
|---|---|---|---|---|---|---|
| Group | | | Animals | Weight | IR | P |
| Number | Group Name | Treatment | Surviving | (x ± SD) | (TV) % | value |
| 1 | Vehicle | PBS | 10 | 3.5911 ± 1.5089 | | |
| | Control | | | | | |
| 2 | Positive | CTX 20 mg/kg, i.p., every | 10 | 1.4398 ± 0.4698 | 59.91 | 0.0004 |
| | Control 1 | other day | | | | |
| 3 | Positive | CTX 40 mg/kg, i.p., every | 10 | 0.1222 ± 0.2207 | 96.60 | 0.0000 |
| | Control 2 | other day | | | | |
| 4 | Test Article 1 | SCV-07, 5 mg/kg, s.c., | 10 | 2.0853 ± 0.5227 | 41.93 | 0.0080 |
| | | daily | | | | |
| 5 | Test Article 2 | SCV-07, 10 mg/kg, s.c., | 10 | 1.7460 ± 0.4757 | 51.38 | 0.0017 |
| | | daily | | | | |
| 6 | Combination 1 | SCV-07, 5 mg/kg, s.c., | 10 | 1.7240 ± 0.6097 | 51.99 | 0.0019 |
| | | daily + CTX 20 mg/kg, | | | | |
| | | i.p., every other day | | | | |
| 7 | Combination 2 | SCV-07, 10 mg/kg, s.c., | 10 | 1.1728 ± 0.5247 | 67.34 | 0.0001 |
| | | daily + CTX 20 mg/kg, | | | | |
| | | i.p., every other day | | | | |

TABLE 7
Statistical results of tumor weights on Day 16

| | | | Number of | Average | | |
|---|---|---|---|---|---|---|
| Group | | | Animals | Weight | IR | P |
| Number | Group Name | Treatment | Surviving | (x ± SD) | (TW) | value |
| 1 | Vehicle | PBS | 10 | 4.062 ± 1.0113 | | |
| | Control | | | | | |
| 2 | Positive | CTX 20 mg/kg, i.p., every | 10 | 1.806 ± 0.5407 | 55.54 | 0.0000 |
| | Control 1 | other day | | | | |
| 3 | Positive | CTX 40 mg/kg, i.p., every | 10 | 0.396 ± 0.5509 | 90.25 | 0.0000 |
| | Control 2 | other day | | | | |
| 4 | Test Article 1 | SCV-07, 5 mg/kg, s.c., | 10 | 3.329 ± 0.6713 | 18.05 | 0.0722 |
| | | daily | | | | |
| 5 | Test Article 2 | SCV-07, 10 mg/kg, s.c., | 10 | 2.819 ± 0.7946 | 30.60 | 0.0068 |
| | | daily | | | | |
| 6 | Combination 1 | SCV-07, 5 mg/kg, s.c., | 10 | 2.089 ± 0.7854 | 48.57 | 0.0001 |
| | | daily + CTX 20 mg/kg, i.p., | | | | |
| | | every other day | | | | |
| 7 | Combination 2 | SCV-07, 10 mg/kg, s.c., | 10 | 1.518 + 0.6677 | 62.63 | 0.0000 |
| | | daily + CTX 20 mg/kg, i.p., | | | | |
| | | every other day | | | | |

TABLE 8
Statistical results of body weights on Day 0

| | | | Number of | Average | | |
|---|---|---|---|---|---|---|
| Group | | | Animals | Weight | IR | P |
| Number | Group Name | Treatment | Surviving | (x ± SD) | (BW) % | value |
| 1 | Vehicle | PBS | 10 | 20.75 ± 1.2869 | | |
| | Control | | | | | |
| 2 | Positive | CTX 20 mg/kg, i.p., | 10 | 20.86 ± 1.2267 | −0.53 | 0.8471 |
| | Control 1 | every other day | | | | |
| 3 | Positive | CTX 40 mg/kg, i.p., | 10 | 20.74 ± 1.0490 | 0.05 | 0.9850 |
| | Control 2 | every other day | | | | |
| 4 | Test Article 1 | SCV-07, 5 mg/kg, | 10 | 20.62 ± 1.3935 | 0.63 | 0.8309 |
| | | s.c., daily | | | | |
| 5 | Test Article 2 | SCV-07, 10 mg/kg, | 10 | 20.84 ± 1.2267 | −0.43 | 0.8746 |
| | | s.c., daily | | | | |
| 6 | Combination 1 | SCV-07, 5 mg/kg, | 10 | 20.62 ± 1.3139 | 0.63 | 0.8256 |
| | | s.c., daily + CTX 20 mg/kg, | | | | |
| | | i.p., every | | | | |
| | | other day | | | | |
| 7 | Combination 2 | SCV-07, 10 mg/kg, | 10 | 20.75 ± 1.2704 | 0.00 | 1.0000 |
| | | s.c., daily + CTX 20 mg/kg, | | | | |
| | | i.p., every | | | | |
| | | other day | | | | |

TABLE 9
Statistical results of body weights on Day 3

| | | | Number of | Average | | |
|---|---|---|---|---|---|---|
| Group | | | Animals | Weight | IR | P |
| Number | Group Name | Treatment | Surviving | (x ± SD) | (BW) % | value |
| 1 | Vehicle | PBS | 10 | 20.56 ± 1.5742 | | |
| | Control | | | | | |
| 2 | Positive | CTX 20 mg/kg, i.p., every | 10 | 20.36 ± 1.3954 | 0.97 | 0.7671 |
| | Control 1 | other day | | | | |
| 3 | Positive | CTX 40 mg/kg, i.p., every | 10 | 19.80 ± 1.0414 | 3.70 | 0.2191 |
| | Control 2 | other day | | | | |
| 4 | Test Article 1 | SCV-07, 5 mg/kg, s.c., | 10 | 20.14 ± 1.3468 | 2.04 | 0.5295 |
| | | daily | | | | |
| 5 | Test Article 2 | SCV-07, 10 mg/kg, s.c., | 10 | 20.08 ± 1.2444 | 2.33 | 0.4592 |
| | | daily | | | | |
| 6 | Combination 1 | SCV-07, 5 mg/kg, s.c., | 10 | 19.98 ± 1.4328 | 2.82 | 0.4002 |
| | | daily + CTX 20 mg/kg, | | | | |
| | | i.p., every other day | | | | |
| 7 | Combination 2 | SCV-07, 10 mg/kg, s.c., | 10 | 20.17 ± 1.6446 | 1.90 | 0.5946 |
| | | daily + CTX 20 mg/kg, | | | | |
| | | i.p., every other day | | | | |

TABLE 10
Statistical results of body weights on Day 6

| | | | Number of | Average | | |
|---|---|---|---|---|---|---|
| Group | | | Animals | Weight | IR | P |
| Number | Group Name | Treatment | Surviving | (x ± SD) | (BW) % | Value |
| 1 | Vehicle | PBS | 10 | 20.51 ± 1.5581 | | |
| | Control | | | | | |
| 2 | Positive | CTX 20 mg/kg, i.p., every | 10 | 18.89 ± 2.0041 | 7.90 | 0.0587 |
| | Control 1 | other day | | | | |
| 3 | Positive | CTX 40 mg/kg, i.p., every | 10 | 18.29 ± 1.5344 | 10.82 | 0.0049 |
| | Control 2 | other day | | | | |
| 4 | Test Article 1 | SCV-07, 5 mg/kg, s.c., | 10 | 20.33 ± 1.6674 | 0.88 | 0.8059 |
| | | daily | | | | |
| 5 | Test Article 2 | SCV-07, 10 mg/kg, s.c., | 10 | 20.44 ± 1.4909 | 0.34 | 0.9194 |
| | | daily | | | | |
| 6 | Combination 1 | SCV-07, 5 mg/kg, s.c., | 10 | 19.59 ± 1.1921 | 4.49 | 0.1554 |
| | | daily + CTX 20 mg/kg, | | | | |
| | | i.p., every other day | | | | |
| 7 | Combination 2 | SCV-07, 10 mg/kg, s.c., | 10 | 19.58 ± 2.3766 | 4.53 | 0.3144 |
| | | daily + CTX 20 mg/kg, | | | | |
| | | i.p., every other day | | | | |

TABLE 11
Statistical results of body weights on Day 9

| | | | Number of | Average | | |
|---|---|---|---|---|---|---|
| Group | | | Animals | Weight | IR | P |
| Number | Group Name | Treatment | Surviving | (x ± SD) | (BW) % | value |
| 1 | Vehicle | PBS | 10 | 22.45 ± 1.0835 | | |
| | Control | | | | | |
| 2 | Positive | CTX 20 mg/kg, i.p., every | 10 | 19.68 ± 2.1856 | 12.35 | 0.0024 |
| | Control 1 | other day | | | | |
| 3 | Positive | CTX 40 mg/kg, i.p., every | 10 | 18.83 ± 1.9922 | 16.12 | 0.0001 |
| | Control 2 | other day | | | | |
| 4 | Test Article 1 | SCV-07, 5 mg/kg, s.c., | 10 | 21.96 ± 1.5551 | 2.18 | 0.4243 |
| | | daily | | | | |
| 5 | Test Article 2 | SCV-07, 10 mg/kg, s.c., | 10 | 21.42 ± 1.7453 | 4.59 | 0.1303 |
| | | daily | | | | |
| 6 | Combination 1 | SCV-07, 5 mg/kg, s.c., | 10 | 20.83 ± 1.1016 | 7.22 | 0.0038 |
| | | daily + CTX 20 mg/kg, | | | | |
| | | i.p., every other day | | | | |
| 7 | Combination 2 | SCV-07, 10 mg/kg, s.c., | 10 | 21.14 ± 2.1966 | 5.84 | 0.1080 |
| | | daily + CTX 20 mg/kg, | | | | |
| | | i.p., every other day | | | | |

TABLE 12
Statistical results of body weights on Day 12

| | | | Number of | Average | | |
|---|---|---|---|---|---|---|
| Group | | | Animals | Weight | IR | P |
| Number | Group Name | Treatment | Surviving | (x ± SD) | (BW) % | value |
| 1 | Vehicle | PBS | 10 | 23.26 ± 2.1665 | | |
| | Control | | | | | |
| 2 | Positive | CTX 20 mg/kg, i.p., every | 10 | 19.81 ± 2.1231 | 14.83 | 0.0021 |
| | Control 1 | other day | | | | |
| 3 | Positive | CTX 40 mg/kg, i.p., every | 10 | 18.15 ± 1.3168 | 21.97 | 0.0000 |
| | Control 2 | other day | | | | |
| 4 | Test Article 1 | SCV-07, 5 mg/kg, s.c., | 10 | 22.21 ± 1.8071 | 4.51 | 0.2545 |
| | | daily | | | | |
| 5 | Test Article 2 | SCV-07, 10 mg/kg, s.c., | 10 | 21.97 ± 1.6371 | 5.55 | 0.1504 |
| | | daily | | | | |
| 6 | Combination 1 | SCV-07, 5 mg/kg, s.c., | 10 | 20.87 ± 1.2623 | 10.28 | 0.0075 |
| | | daily + CTX 20 mg/kg, | | | | |
| | | i.p., every other day | | | | |
| 7 | Combination 2 | SCV-07, 10 mg/kg, s.c., | 10 | 20.98 ± 2.4512 | 9.80 | 0.0408 |
| | | daily + CTX 20 mg/kg, | | | | |
| | | i.p., every other day | | | | |

TABLE 13
Statistical results of body weights on Day 15

| | | | Number of | Average | | |
|---|---|---|---|---|---|---|
| Group | | | Animals | Weight | IR | P |
| Number | Group Name | Treatment | Surviving | (x ± SD) | (BW) % | value |
| 1 | Vehicle | PBS | 10 | 24.01 ± 1.9076 | | |
| | Control | | | | | |
| 2 | Positive | CTX 20 mg/kg, i.p., every | 10 | 22.75 ± 2.3590 | 5.25 | 0.2056 |
| | Control 1 | other day | | | | |
| 3 | Positive | CTX 40 mg/kg, i.p., every | 10 | 17.98 ± 1.9240 | 25.11 | 0.0000 |
| | Control 2 | other day | | | | |
| 4 | Test Article 1 | SCV-07, 5 mg/kg, s.c., | 10 | 23.05 ± 2.1423 | 4.00 | 0.3039 |
| | | daily | | | | |
| 5 | Test Article 2 | SCV-07, 10 mg/kg, s.c., | 10 | 23.19 ± 1.5538 | 3.42 | 0.3058 |
| | | daily | | | | |
| 6 | Combination 1 | SCV-07, 5 mg/kg, s.c., | 10 | 22.52 ± 1.4695 | 6.21 | 0.0661 |
| | | daily + CTX 20 mg/kg, | | | | |
| | | i.p., every other day | | | | |
| 7 | Combination 2 | SCV-07, 10 mg/kg, s.c., | 10 | 22.43 ± 2.3641 | 6.58 | 0.1174 |
| | | daily + CTX 20 mg/kg, | | | | |
| | | i.p., every other day | | | | |

APPENDIX 1
Tumor measurements (cm) on Day 3

| Group | Treatment | F1 | | F2 | | F3 | | F4 | | F5 | | M1 | | M2 | | M3 | | M4 | | M5 | |
|---|---|---|---|---|---|---|---|---|---|---|---|---|---|---|---|---|---|---|---|---|---|
| 1 | Vehicle Control | 0.3 | 0.3 | 0.4 | 0.4 | 0.3 | 0.3 | 0.4 | 0.4 | 0.3 | 0.3 | 0.3 | 0.3 | 0.3 | 0.3 | 0.3 | 0.3 | 0.3 | 0.3 | 0.3 | 0.3 |
| 2 | CTX 20 mg/kg | — | — | — | — | 0.3 | 0.3 | — | — | — | — | — | — | 0.2 | 0.2 | 0.2 | 0.2 | — | — | 0.1 | 0.1 |
| 3 | CTX 40 mg/kg | — | — | — | — | 0.3 | 0.3 | 0.1 | 0.1 | — | — | — | — | — | — | — | — | — | — | — | — |
| 4 | SCV-07 5 mg/kg | 0.4 | 0.4 | 0.3 | 0.3 | 0.4 | 0.4 | — | — | 0.3 | 0.3 | 0.4 | 0.4 | — | — | 0.2 | 0.2 | 0.3 | 0.3 | 0.4 | 0.4 |
| 5 | SCV-07 10 mg/kg | 0.3 | 0.3 | 0.3 | 0.3 | 0.3 | 0.3 | — | — | | | 0.3 | 0.3 | 0.2 | 0.2 | — | — | 0.3 | 0.3 | — | — |
| 6 | SCV-07 5 mg/kg + | 0.4 | 0.4 | 0.2 | 0.2 | 0.3 | 0.3 | — | — | 0.3 | 0.3 | 0.3 | 0.3 | 0.2 | 0.2 | — | — | 0.3 | 0.3 | — | — |
| | CTX 20 mg/kg | | | | | | | | | | | | | | | | | | | | |
| 7 | SCV-07 10 mg/kg + | 0.2 | 0.2 | 0.2 | 0.2 | 0.2 | 0.2 | 0.3 | 0.3 | — | — | 0.3 | 0.3 | — | — | 0.3 | 0.3 | 0.3 | 0.3 | 0.2 | 0.2 |
| | CTX 20 mg/kg | | | | | | | | | | | | | | | | | | | | |

— Not measurable

APPENDIX 2
Tumor volumes* (cm³) on Day 3

| Group | Treatment | F1 | F2 | F3 | F4 | F5 | M1 | M2 | M3 | M4 | M5 |
|---|---|---|---|---|---|---|---|---|---|---|---|
| 1 | Vehicle Control | 0.0135 | 0.032 | 0.0135 | 0.032 | 0.0135 | 0.0135 | 0.0135 | 0.0135 | 0.0135 | 0.0135 |
| 2 | CTX 20 mg/kg | 0 | 0 | 0.0135 | 0 | 0 | 0 | 0.004 | 0.004 | 0 | 0.0005 |
| 3 | CTX 40 mg/kg | 0 | 0 | 0.0135 | 0.0005 | 0 | 0 | 0 | 0 | 0 | 0 |
| 4 | SCV-07 5 mg/kg | 0.032 | 0.0135 | 0.032 | 0 | 0.0135 | 0.032 | 0 | 0.004 | 0.0135 | 0.032 |
| 5 | SCV-07 10 mg/kg | 0.0135 | 0.0135 | 0.0135 | 0 | 0 | 0.0135 | 0.004 | 0 | 0.0135 | 0 |
| 6 | SCV-07 5 mg/kg + | 0.032 | 0.004 | 0.0135 | 0 | 0.0135 | 0.0135 | 0.004 | 0 | 0.0135 | 0 |
| | CTX 20 mg/kg | | | | | | | | | | |
| 7 | SCV-07 10 mg/kg + | 0.004 | 0.004 | 0.004 | 0.0135 | 0 | 0.0135 | 0 | 0.0135 | 0.0135 | 0.004 |
| | CTX 20 mg/kg | | | | | | | | | | |

*Tumor volumes were calculated using the formula “Tumor Volume = Length × Width × Width/2” based on the data listed in Appendix 1.

APPENDIX 3
Tumor measurements (cm) on Day 6

| | | F1 | | F2 | | F3 | | F4 | | F5 | | M1 | | M2 | | M3 | | M4 | | M5 | |
|---|---|---|---|---|---|---|---|---|---|---|---|---|---|---|---|---|---|---|---|---|---|
| Group | Treatment | L | W | L | W | L | W | L | W | L | W | L | W | L | W | L | W | L | W | L | W |
| 1 | Vehicle Control | 0.5 | 0.5 | 0.4 | 0.4 | 0.5 | 0.5 | 0.5 | 0.5 | 0.5 | 0.5 | 0.8 | 0.8 | 0.8 | 0.8 | 0.5 | 0.5 | 0.7 | 0.7 | 0.5 | 0.5 |
| 2 | CTX 20 mg/kg | 0.3 | 0.3 | | | 0.3 | 0.3 | | | | | 0.4 | 0.4 | 0.4 | 0.4 | 0.5 | 0.5 | 0.1 | 0.1 | 0.5 | 0.5 |
| 3 | CTX 40 mg/kg | | | | | 0.5 | 0.5 | 0.2 | 0.2 | | | | | | | | | | | 0.1 | 0.1 |
| 4 | SCV-07 5 mg/kg | 0.6 | 0.6 | 0.6 | 0.4 | 0.5 | 0.5 | 0.6 | 0.6 | 0.6 | 0.6 | 0.6 | 0.6 | 0.5 | 0.5 | 0.6 | 0.6 | 0.6 | 0.6 | 0.5 | 0.5 |
| 5 | SCV-07 10 mg/kg | 0.5 | 0.5 | 0.5 | 0.5 | 0.5 | 0.5 | 0.5 | 0.5 | 0.6 | 0.6 | 0.6 | 0.6 | 0.6 | 0.6 | 0.5 | 0.5 | 0.7 | 0.7 | 0.5 | 0.5 |
| 6 | SCV-07 5 mg/kg + | 0.4 | 0.4 | 0.6 | 0.6 | 0.5 | 0.5 | 0.3 | 0.3 | 0.5 | 0.5 | 0.7 | 0.7 | 0.7 | 0.7 | 0.4 | 0.4 | 0.6 | 0.6 | 0.5 | 0.5 |
| | CTX 20 mg/kg | | | | | | | | | | | | | | | | | | | | |
| 7 | SCV-07 10 mg/kg + | 0.5 | 0.5 | 0.6 | 0.6 | 0.3 | 0.3 | 0.5 | 0.5 | | | 0.5 | 0.5 | 0.3 | 0.3 | 0.6 | 0.6 | 0.6 | 0.6 | 0.5 | 0.5 |
| | CTX 20 mg/kg | | | | | | | | | | | | | | | | | | | | |

— Not measurable

APPENDIX 4
Tumor volumes* (cm³) on Day 6

| Group | Treatment | F1 | F2 | F3 | F4 | F5 | M1 | M2 | M3 | M4 | M5 |
|---|---|---|---|---|---|---|---|---|---|---|---|
| 1 | Vehicle Control | 0.0625 | 0.032 | 0.0625 | 0.0625 | 0.0625 | 0.256 | 0.256 | 0.0625 | 0.1715 | 0.0625 |
| 2 | CTX 20 mg/kg | 0.0135 | 0 | 0.0135 | 0 | 0 | 0.032 | 0.032 | 0.0625 | 0.0005 | 0.0625 |
| 3 | CTX 40 mg/kg | 0 | 0 | 0.0625 | 0.004 | 0 | 0 | 0 | 0 | 0 | 0.0005 |
| 4 | SCV-07 5 mg/kg | 0.108 | 0.072 | 0.0625 | 0.108 | 0.108 | 0.108 | 0.0625 | 0.108 | 0.108 | 0.0625 |
| 5 | SCV-07 10 mg/kg | 0.0625 | 0.0625 | 0.0625 | 0.0625 | 0.108 | 0.108 | 0.108 | 0.0625 | 0.1715 | 0.0625 |
| 6 | SCV-07 5 mg/kg + | 0.032 | 0.108 | 0.0625 | 0.0135 | 0.0625 | 0.1715 | 0.1715 | 0.032 | 0.108 | 0.0625 |
| | CTX 20 mg/kg | | | | | | | | | | |
| 7 | SCV-07 10 mg/kg + | 0.0625 | 0.108 | 0.0135 | 0.0625 | 0 | 0.0625 | 0.0135 | 0.108 | 0.108 | 0.0625 |
| | CTX 20 mg/kg | | | | | | | | | | |

*Tumor volumes were calculated using the formula “Tumor Volume = Length × Width × Width/2” based on the data listed in Appendix 3.

APPENDIX 5
Tumor measurements (cm) on Day 9

| | | F1 | | F2 | | F3 | | F4 | | F5 | | M1 | | M2 | | M3 | | M4 | | M5 | |
|---|---|---|---|---|---|---|---|---|---|---|---|---|---|---|---|---|---|---|---|---|---|
| Group | Treatment | L | W | L | W | L | W | L | W | L | W | L | W | L | W | L | W | L | W | L | W |
| 1 | Vehicle Control | 0.8 | 0.8 | 0.9 | 0.9 | 1.2 | 1.1 | 0.9 | 0.9 | 0.8 | 0.8 | 1.2 | 1.2 | 1.3 | 1.3 | 0.8 | 0.8 | 1.2 | 1.2 | 1.0 | 1.0 |
| 2 | CTX 20 mg/kg | 0.5 | 0.5 | 0.5 | 0.5 | 0.5 | 0.5 | 0.5 | 0.5 | 0.5 | 0.5 | 0.5 | 0.5 | 0.5 | 0.4 | 0.5 | 0.5 | 0.4 | 0.4 | 0.5 | 0.5 |
| 3 | CTX 40 mg/kg | 0.2 | 0.2 | | | 0.5 | 0.5 | 0.3 | 0.3 | 0.2 | 0.2 | | | | | | | | | 0.1 | 0.1 |
| 4 | SCV-07 5 mg/kg | 1 | 0.5 | 1 | 0.7 | 1.1 | 1.1 | 1.2 | 1.1 | 0.9 | 0.9 | 1.2 | 1.2 | 1 | 1 | 1 | 1 | 1 | 1 | 1 | 1 |
| 5 | SCV-07 10 mg/kg | 0.8 | 0.8 | 0.7 | 0.7 | 0.9 | 0.9 | 0.9 | 0.9 | 1 | 1 | 1.0 | 1.0 | 1.0 | 1.0 | 0.8 | 0.8 | 1.2 | 1.2 | 0.8 | 0.7 |
| 6 | SCV-07 5 mg/kg + | 0.8 | 0.8 | 1.1 | 1 | 0.7 | 0.7 | 0.7 | 0.7 | 1 | 1 | 0.8 | 0.8 | 1 | 1 | 0.7 | 0.7 | 0.7 | 0.7 | 0.7 | 0.7 |
| | CTX 20 mg/kg | | | | | | | | | | | | | | | | | | | | |
| 7 | SCV-07 10 mg/kg + | 0.5 | 0.5 | 0.7 | 0.7 | 0.4 | 0.4 | 0.6 | 0.6 | 0.2 | 0.1 | 0.5 | 0.5 | 0.6 | 0.6 | 0.7 | 0.7 | 0.7 | 0.7 | 0.6 | 0.6 |
| | CTX 20 mg/kg | | | | | | | | | | | | | | | | | | | | |

— Not measurable

APPENDIX 6
Tumor volumes* (cm³) on Day 9

| Group | Treatment | F1 | F2 | F3 | F4 | F5 | M1 | M2 | M3 | M4 | M5 |
|---|---|---|---|---|---|---|---|---|---|---|---|
| 1 | Vehicle Control | 0.256 | 0.3645 | 0.792 | 0.3645 | 0.256 | 0.5 | 1.0985 | 0.256 | 0.864 | 0.5 |
| 2 | CTX 40 mg/kg | 0.0625 | 0.0625 | 0.0625 | 0.0625 | 0.0625 | 0.0625 | 0.05 | 0.0625 | 0.032 | 0.0625 |
| 3 | SCV-07 0.01 mg/kg | 0.004 | 0 | 0.0625 | 0.0135 | 0.004 | 0 | 0 | 0 | 0 | 0.0005 |
| 4 | SCV-07 0.1 mg/kg | 0.25 | 0.35 | 0.6655 | 0.792 | 0.3645 | 0.864 | 0.5 | 0.5 | 0.5 | 0.5 |
| 5 | SCV-07 1.0 mg/kg | 0.256 | 0.1715 | 0.3645 | 0.3645 | 0.5 | 0.5 | 0.5 | 0.256 | 0.864 | 0.224 |
| 6 | SCV-07 5 mg/kg + | 0.605 | 0.605 | 0.1715 | 0.1715 | 0.5 | 0.256 | 0.5 | 0.1715 | 0.1715 | 0.1715 |
| | CTX 20 mg/kg | | | | | | | | | | |
| 7 | SCV-07 10 mg/kg + | 0.0625 | 0.1715 | 0.032 | 0.108 | 0.002 | 0.0625 | 0.108 | 0.1715 | 0.1715 | 0.108 |
| | CTX 20 mg/kg | | | | | | | | | | |

*Tumor volumes were calculated using the formula “Tumor Volume = Length × Width × Width/2” based on the data listed in Appendix 5.

APPENDIX 7
Tumor measurements (cm) on Day 12

| | | F1 | | F2 | | F3 | | F4 | | F5 | | M1 | | M2 | | M3 | | M4 | | M5 | |
|---|---|---|---|---|---|---|---|---|---|---|---|---|---|---|---|---|---|---|---|---|---|
| Group | Treatment | L | W | L | W | L | W | L | W | L | W | L | W | L | W | L | W | L | W | L | W |
| 1 | Vehicle Control | 1.2 | 1.2 | 1.2 | 1.2 | 1.7 | 1.7 | 1.5 | 1.5 | 1.5 | 1.3 | 1.7 | 1.7 | 1.6 | 1.5 | 1.2 | 1.2 | 1.7 | 1.7 | 1.5 | 1.4 |
| 2 | CTX 20 mg/kg | 0.9 | 0.9 | 1.1 | 1.1 | 0.8 | 0.8 | 1 | 1 | 0.8 | 0.8 | 1 | 1 | 1 | 1 | 1.1 | 1.1 | 0.9 | 0.9 | 1.2 | 1.1 |
| 3 | CTX 40 mg/kg | 0.3 | 0.3 | — | — | 0.6 | 0.6 | 0.3 | 0.3 | 0.3 | 0.3 | — | — | — | — | — | — | — | — | — | — |
| 4 | SCV-07 5 mg/kg | 1.2 | 1 | 1.5 | 1.2 | 1.3 | 1.3 | 1.3 | 1.3 | 1.3 | 1.3 | 1.5 | 1.3 | 1.5 | 1.2 | 1.3 | 1.3 | 1.3 | 1.3 | 1.4 | 1.3 |
| 5 | SCV-07 10 mg/kg | 1.2 | 1.2 | 1.1 | 1.1 | 1. | 1 | 1.2 | 1.2 | 1.2 | 1.2 | 1.2 | 1 | 1.2 | 1.2 | 1 | 1 | 1.3 | 1.3 | 0.8 | 0.8 |
| 6 | SCV-07 5 mg/kg + | 1 | 1 | 1.6 | 1.2 | 1 | 1 | 1 | 1 | 1 | 1 | 1.1 | 1.1 | 1.4 | 1.3 | 1.1 | 1.1 | 1.2 | 1.2 | 1 | 1 |
| | CTX 20 mg/kg | | | | | | | | | | | | | | | | | | | | |
| 7 | SCV-07 10 mg/kg + | 0.9 | 0.9 | 1 | 1 | 0.5 | 0.5 | 1 | 1 | 0.3 | 0.3 | 0.8 | 0.8 | 0.8 | 0.8 | 1.2 | 1.2 | 1 | 1 | 0.8 | 0.8 |
| | CTX 20 mg/kg | | | | | | | | | | | | | | | | | | | | |

— not measurable

APPENDIX 8
Tumor volumes* (cm³) on Day 12

| Group | Treatment | F1 | F2 | F3 | F4 | F5 | M1 | M2 | M3 | M4 | M5 |
|---|---|---|---|---|---|---|---|---|---|---|---|
| 1 | Vehicle Control | 0.864 | 0.864 | 2.4565 | 1.6875 | 1.4625 | 2.4565 | 1.92 | 0.864 | 2.4565 | 1.575 |
| 2 | CTX 20 mg/kg | 0.3645 | 0.6655 | 0.256 | 0.5 | 0.256 | 0.5 | 0.5 | 0.6655 | 0.3645 | 0.792 |
| 3 | CTX 40 mg/kg | 0.0135 | 0 | 0.108 | 0.0135 | 0.0135 | 0 | 0 | 0 | 0 | 0 |
| 4 | SCV-07 5 mg/kg | 0.72 | 1.35 | 1.0985 | 1.0985 | 1.0985 | 1.4625 | 1.35 | 1.0985 | 1.0985 | 1.274 |
| 5 | SCV-07 10 mg/kg | 0.864 | 0.6655 | 0.5 | 0.864 | 0.864 | 0.72 | 0.864 | 0.5 | 1.0985 | 0.256 |
| 6 | SCV-07 5 mg/kg + | 0.5 | 1.536 | 0.5 | 0.5 | 0.5 | 0.6655 | 1.274 | 0.6655 | 0.864 | 0.5 |
| | CTX 20 mg/kg | | | | | | | | | | |
| 7 | SCV-07 10 mg/kg + | 0.3645 | 0.5 | 0.0625 | 0.5 | 0.0135 | 0.256 | 0.256 | 0.864 | 0.5 | 0.256 |
| | CTX 20 mg/kg | | | | | | | | | | |

*Tumor volumes were calculated using the formula “Tumor Volume = Length × Width × Width/2” based on the data listed in Appendix 7.

APPENDIX 9
Tumor measurements (cm) on Day 15

| | | F1 | | F2 | | F3 | | F4 | | F5 | | M1 | | M2 | | M3 | | M4 | | M5 | |
|---|---|---|---|---|---|---|---|---|---|---|---|---|---|---|---|---|---|---|---|---|---|
| Group | Treatment | L | W | L | W | L | W | L | W | L | W | L | W | L | W | L | W | L | W | L | W |
| 1 | Vehicle Control | 1.5 | 1.5 | 1.8 | 1.8 | 2 | 1.7 | 2.4 | 1.8 | 2 | 1.5 | 2.4 | 2 | 1.8 | 1.8 | 1.5 | 1.5 | 2.4 | 2 | 2 | 1.8 |
| 2 | CTX 20 mg/kg | 1.4 | 1.3 | 1.6 | 1.2 | 1.3 | 1.2 | 1.5 | 1.5 | 1.7 | 1.5 | 1.3 | 1.3 | 1.2 | 1.2 | 1.7 | 1.5 | 1.3 | 1.2 | 1.5 | 1.4 |
| 3 | CTX 40 mg/kg | 0.5 | 0.5 | | | 1.1 | 1.1 | 0.9 | 0.9 | 0.5 | 0.5 | | | 0.5 | 0.5 | | | | | 0.2 | 0.2 |
| 4 | SCV-07 5 mg/kg | 1.7 | 1.4 | 1.7 | 1.1 | 1.7 | 1.4 | 1.7 | 1.7 | 1.4 | 1.4 | 2 | 1.5 | 1.8 | 1.5 | 1.5 | 1.4 | 1.6 | 1.4 | 1.8 | 1.6 |
| 5 | SCV-07 10 mg/kg | 1.4 | 1.4 | 1.7 | 1.7 | 1.7 | 1.7 | 1.4 | 1.4 | 1.4 | 1.4 | 1.5 | 1.5 | 1.4 | 1.4 | 1.5 | 1.5 | 1.7 | 1.6 | 1.4 | 1.4 |
| 6 | SCV-07 5 mg/kg + | 1.2 | 1.2 | 1.7 | 1.6 | 1.2 | 1.2 | 1.4 | 1.4 | 1.5 | 1.5 | 1.7 | 1.7 | 1.7 | 1.7 | 1.7 | 1.5 | 1.5 | 1.5 | 1.4 | 1.4 |
| | CTX 20 mg/kg | | | | | | | | | | | | | | | | | | | | |
| 7 | SCV-07 10 mg/kg + | 1.4 | 1.4 | 1.4 | 1.4 | 1 | 1 | 1.3 | 1.3 | 0.5 | 0.5 | 1.4 | 1.3 | 1.5 | 1.5 | 1.5 | 1.4 | 1.5 | 1.5 | 1.3 | 1.3 |
| | CTX 20 mg/kg | | | | | | | | | | | | | | | | | | | | |

APPENDIX 10
Tumor volumes* (cm³) on Day 15

| Group | Treatment | F1 | F2 | F3 | F4 | F5 | M1 | M2 | M3 | M4 | M5 |
|---|---|---|---|---|---|---|---|---|---|---|---|
| 1 | Vehicle Control | 1.6875 | 2.916 | 3.4 | 5.184 | 3 | 5.76 | 2.916 | 1.6875 | 5.76 | 3.6 |
| 2 | CTX 20 mg/kg | 1.274 | 1.536 | 1.014 | 1.6875 | 2.1675 | 1.0985 | 0.864 | 2.1675 | 1.014 | 1.575 |
| 3 | CTX 40 mg/kg | 0.0625 | 0 | 0.6655 | 0.3645 | 0.0625 | 0 | 0.0625 | 0 | 0 | 0.004 |
| 4 | SCV-07 5 mg/kg | 2.023 | 1.5895 | 2.023 | 2.4565 | 1.372 | 3 | 2.43 | 1.575 | 1.792 | 2.592 |
| 5 | SCV-07 10 mg/kg | 1.372 | 2.4565 | 2.4565 | 1.372 | 1.372 | 1.6875 | 1.372 | 1.6875 | 2.312 | 1.372 |
| 6 | SCV-07 5 mg/kg + | 0.864 | 2.312 | 0.864 | 1.372 | 1.6875 | 2.4565 | 2.4565 | 2.1675 | 1.6875 | 1.372 |
| | CTX 20 mg/kg | | | | | | | | | | |
| 7 | SCV-07 10 mg/kg + | 1.372 | 1.372 | 0.5 | 1.0985 | 0.0625 | 1.274 | 1.6875 | 1.575 | 1.6875 | 1.0985 |
| | CTX 20 mg/kg | | | | | | | | | | |

*Tumor volumes were calculated using the formula “Tumor Volume = Length × Width × Width/2” based on the data listed in Appendix 9.

APPENDIX 11
Tumor weight* (g) on Day 16

| Group | Treatment | F1 | F2 | F3 | F4 | F5 | M1 | M2 | M3 | M4 | M5 |
|---|---|---|---|---|---|---|---|---|---|---|---|
| 1 | Vehicle Control | 3.01 | 3.61 | 4.49 | 4.20 | 2.90 | 5.34 | 5.33 | 2.79 | 5.23 | 3.72 |
| 2 | CTX 20 mg/kg | 2.05 | 1.64 | 1.44 | 1.72 | 1.72 | 1.63 | 1.43 | 2.90 | 1.05 | 2.48 |
| 3 | CTX 40 mg/kg | 0.29 | 0.12 | 1.80 | 0.84 | 0.39 | 0.28 | 0.02 | 0.03 | 0.15 | 0.04 |
| 4 | SCV-07 5 mg/kg | 2.87 | 2.05 | 3.23 | 4.03 | 2.94 | 4.40 | 3.18 | 3.11 | 3.71 | 3.77 |
| 5 | SCV-07 10 mg/kg | 2.86 | 2.96 | 4.03 | 2.05 | 2.7 | 3.18 | 2.20 | 2.42 | 4.10 | 1.69 |
| 6 | SCV-07 5 mg/kg + | 1.60 | 3.40 | 1.26 | 1.60 | 1.40 | 2.46 | 3.39 | 2.07 | 2.21 | 1.50 |
| | CTX 20 mg/kg | | | | | | | | | | |
| 7 | SCV-07 10 mg/kg + | 1.57 | 2.00 | 0.56 | 2.53 | 0.47 | 0.99 | 1.67 | 2.00 | 1.96 | 1.43 |
| | CTX 20 mg/kg | | | | | | | | | | |

APPENDIX 12
Body weights (g) on Day 0

| Group | Treatment | F1 | F2 | F3 | F4 | F5 | M1 | M2 | M3 | M4 | M5 |
|---|---|---|---|---|---|---|---|---|---|---|---|
| 1 | Vehicle Control | 19.5 | 19.4 | 19.7 | 19.5 | 19.6 | 21.9 | 21.8 | 22.2 | 22.2 | 21.7 |
| 2 | CTX 20 mg/kg | 19.8 | 19.7 | 19.1 | 20.2 | 19.9 | 22.2 | 22.1 | 21.5 | 22.2 | 21.9 |
| 3 | CTX 40 mg/kg | 19.3 | 20.1 | 19.5 | 19.7 | 20.3 | 21.7 | 21.7 | 21.7 | 21.7 | 21.7 |
| 4 | SCV-07 5 mg/kg | 19.8 | 19.2 | 19.2 | 19.9 | 18.7 | 21.9 | 21.3 | 22.4 | 22 | 21.8 |
| 5 | SCV-07 10 mg/kg | 19.9 | 19.3 | 19.6 | 19.7 | 20 | 21.9 | 22.2 | 21.6 | 22 | 22.2 |
| 6 | SCV-07 5 mg/kg + | 19.2 | 19.1 | 19.5 | 19.5 | 19.7 | 22.1 | 21.4 | 21.8 | 22.2 | 21.7 |
| | CTX 20 mg/kg | | | | | | | | | | |
| 7 | SCV-07 10 mg/kg + | 20 | 19.5 | 19.6 | 19.4 | 19.4 | 21.6 | 22.4 | 21.5 | 21.9 | 22.2 |
| | CTX 20 mg/kg | | | | | | | | | | |

APPENDIX 13
Body weights (g) on Day 3

| Group | Treatment | F1 | F2 | F3 | F4 | F5 | M1 | M2 | M3 | M4 | M5 |
|---|---|---|---|---|---|---|---|---|---|---|---|
| 1 | Vehicle Control | 19.5 | 18.4 | 19.8 | 19 | 18.9 | 21.9 | 21.6 | 22.4 | 21.9 | 22.2 |
| 2 | CTX 20 mg/kg | 18.7 | 19.6 | 18.6 | 19.7 | 18.9 | 21.5 | 22 | 21.1 | 21.5 | 22 |
| 3 | CTX 40 mg/kg | 18.9 | 19.1 | 18 | 18.8 | 20.1 | 20.1 | 20.5 | 20.5 | 20.7 | 21.3 |
| 4 | SCV-07 5 mg/kg | 19.2 | 18.9 | 19.3 | 19.6 | 17.8 | 21.9 | 21.2 | 21.3 | 21.2 | 21 |
| 5 | SCV-07 10 mg/kg | 18.8 | 19 | 19 | 19 | 19.3 | 21.6 | 22.3 | 20.7 | 20.8 | 20.3 |
| 6 | SCV-07 5 mg/kg + | 18.4 | 18.1 | 19 | 19.1 | 18.9 | 21. | 21.6 | 21.9 | 21.4 | 20.4 |
| | CTX 20 mg/kg | | | | | | | | | | |
| 7 | SCV-07 10 mg/kg + | 19.3 | 18.1 | 19 | 19 | 18.1 | 21.6 | 22 | 22.4 | 21.4 | 20.8 |
| | CTX 20 mg/kg | | | | | | | | | | |

APPENDIX 14
Body weights (g) on Day 6

| Group | Treatment | F1 | F2 | F3 | F4 | F5 | M1 | M2 | M3 | M4 | M5 |
|---|---|---|---|---|---|---|---|---|---|---|---|
| 1 | Vehicle Control | 18.7 | 18.8 | 19.6 | 20 | 18.7 | 21.8 | 21.8 | 22.5 | 22.5 | 20.7 |
| 2 | CTX 20 mg/kg | 17 | 16.6 | 17.3 | 16.7 | 17.8 | 20 | 22 | 20.2 | 20.6 | 20.7 |
| 3 | CTX 40 mg/kg | 17 | 17.6 | 15.8 | 16.7 | 17.8 | 19.7 | 19.3 | 19.4 | 19 | 20.6 |
| 4 | SCV-07 5 mg/kg | 20.4 | 19 | 18.2 | 19.5 | 17.8 | 22.9 | 21.7 | 21.8 | 21.2 | 20.8 |
| 5 | SCV-07 10 mg/kg | 18.5 | 19.6 | 19.1 | 19.2 | 19.5 | 22.2 | 22.9 | 21.7 | 20.5 | 21.2 |
| 6 | SCV-07 5 mg/kg + | 17.8 | 20.1 | 18.4 | 18.4 | 19 | 21.4 | 20.5 | 19.2 | 20.7 | 20.4 |
| | CTX 20 mg/kg | | | | | | | | | | |
| 7 | SCV-07 10 mg/kg + | 16.5 | 18.2 | 17.9 | 16.9 | 17.4 | 21.6 | 21.4 | 22.2 | 22.1 | 21.6 |
| | CTX 20 mg/kg | | | | | | | | | | |

APPENDIX 15
Body weights (g) on Day 9

| Group | Treatment | F1 | F2 | F3 | F4 | F5 | M1 | M2 | M3 | M4 | M5 |
|---|---|---|---|---|---|---|---|---|---|---|---|
| 1 | Vehicle Control | 21.5 | 21.8 | 21.6 | 21.2 | 21.2 | 23.6 | 22.9 | 23.8 | 23.3 | 23.6 |
| 2 | CTX 20 mg/kg | 17.5 | 18.8. | 17. | 17.5 | 18 | 21.4 | 22.5 | 22 | 20 | 21.2 |
| 3 | CTX 40 mg/kg | 17.5 | 19.1 | 15.4 | 17.1 | 17.2 | 20.4 | 20.3 | 18.8 | 21.5 | 21 |
| 4 | SCV-07 5 mg/kg | 21.8 | 21.6 | 19.5 | 20.6 | 20 | 24. | 23.7 | 23 | 23.2 | 22.2 |
| 5 | SCV-07 10 mg/kg | 18.9 | 20.7 | 19.7 | 20.1 | 20.3 | 23.3 | 24.4 | 22.4 | 22.2 | 22.2 |
| 6 | SCV-07 5 mg/kg + | 19.9 | 21.1 | 18.6 | 21.2 | 19.9 | 22 | 22.2 | 20.9 | 20.8 | 21.7 |
| | CTX 20 mg/kg | | | | | | | | | | |
| 7 | SCV-07 10 mg/kg + | 18.3 | 20.8 | 18.9 | 20 | 18.4 | 21.9 | 23.5 | 24.5 | 22.6 | 22.5 |
| | CTX 20 mg/kg | | | | | | | | | | |

APPENDIX 16
Body weights (g) on Day 12

| Group | Treatment | F1 | F2 | F3 | F4 | F5 | M1 | M2 | M3 | M4 | M5 |
|---|---|---|---|---|---|---|---|---|---|---|---|
| 1 | Vehicle Control | 21.5 | 21.5 | 20.7 | 21.8 | 20.8 | 25.7 | 25.0 | 24.4 | 25.8 | 25.4 |
| 2 | CTX 20 mg/kg | 17.9 | 18.3 | 17.9 | 18.3 | 17 | 20.9 | 22.7 | 22.1 | 21.9 | 21.1 |
| 3 | CTX 40 mg/kg | 17.3 | 20 | 16.7 | 17 | 18.2 | 18.5 | 17.4 | 16.8 | 20 | 19.6 |
| 4 | SCV-07 5 mg/kg | 21.7 | 20.8 | 20 | 19.9 | 20.4 | 23.8 | 23.8 | 23.8 | 23.9 | 24 |
| 5 | SCV-07 10 mg/kg | 19.7 | 21.2 | 20.5 | 20.4 | 21.5 | 23.7 | 25 | 23 | 22.4 | 22.3 |
| 6 | SCV-07 5 mg/kg + | 19.2 | 21.7 | 18.9 | 20.7 | 20.2 | 21.5 | 23 | 20.7 | 22.1 | 20.7 |
| | CTX 20 mg/kg | | | | | | | | | | |
| 7 | SCV-07 10 mg/kg + | 17.5 | 19.6 | 18.7 | 19.8 | 18.6 | 22.2 | 23.4 | 24.5 | 23.7 | 21.8 |
| | CTX 20 mg/kg | | | | | | | | | | |

APPENDIX 17
Body weights (g) on Day 15

| Group | Treatment | F1 | F2 | F3 | F4 | F5 | M1 | M2 | M3 | M4 | M5 |
|---|---|---|---|---|---|---|---|---|---|---|---|
| 1 | Vehicle Control | 22.5 | 22.9 | 22.7 | 22.3 | 21.4 | 25.7 | 24.5 | 25.4 | 25.4 | 27.3 |
| 2 | CTX 20 mg/kg | 23.7 | 25.1 | 25.4 | 23.0 | 24.2 | 21.3 | 20.9 | 18.7 | 25.1 | 20.1 |
| 3 | CTX 40 mg/kg | 16.9 | 19.8 | 17.9 | 17.6 | 16.7 | 18.2 | 15.2 | 16.0 | 20.7 | 20.8 |
| 4 | SCV-07 5 mg/kg | 22.4 | 21.3 | 20.3 | 20.5 | 21.8 | 26.3 | 24.6 | 23.0 | 25.6 | 24.7 |
| 5 | SCV-07 10 mg/kg | 20.7 | 22.4 | 22.4 | 21.5 | 22.7 | 24.0 | 26.0 | 24.0 | 24.5 | 23.7 |
| 6 | SCV-07 5 mg/kg + | 20.5 | 23.2 | 20.2 | 22.6 | 21.3 | 23.1 | 25.1 | 23.1 | 23.3 | 22.8 |
| | CTX 20 mg/kg | | | | | | | | | | |
| 7 | SCV-07 10 mg/kg + | 20.8 | 21.3 | 19.8 | 20.5 | 19.0 | 23.6 | 25.1 | 25.4 | 25.0 | 23.6 |
| | CTX 20 mg/kg | | | | | | | | | | |

### Example 2

An Evaluation of SCV-07 in Combination with Radiation for Efficacy in the Reduction of Tumor Growth.

### Summary

In this study, the impact of SCV-07 on tumor growth with or without radiation treatment was tested using the H146 lung cancer model in mice. Tumor bearing mice were treated with saline or SCV-07 with or without radiation therapy once a day for twenty days. SCV-07 showed no evidence of toxicity in this study based on observations of survival and weight change and it did not alter the response of H146 tumors to radiation. When given alone, SCV-07 was effective in reducing tumor growth in a dose dependent manner, with animals receiving SCV-07 twice daily for 20 days at 1 mg/kg showing tumor growth inhibition of 9.1% and animals receiving SCV-07 twice daily for 20 days at 10 mg/kg showing 35.9% tumor growth inhibition. When combined with a single dose of radiation therapy, treatment with SCV-07 at 10 mg/kg twice daily for 20 days resulted in a 78.3% tumor growth inhibition, or a TGI of 40.5% relative to the animals treated with radiation alone. Based on these observations, SCV-07 appears to be effective in reducing the growth of tumors in a lung cancer model when given either alone or in combination with radiation therapy.

### Objective

To evaluate the efficacy of SCV-07 in inhibiting tumor growth using a NCl H146 small cell lung cancer model in mice, both as mono-therapy and in conjunction with radiotherapy.

### Study DESIGN

Ninety-Six (96) female nude mice (nu/nu) were be randomly assigned into 8 treatment groups. Each mouse was inoculated into their lower left flank with 1×10⁵ NCl—H146 (H146) lung cancer cells in a volume of 0.1 mL with Matrigel. Treatment began once tumors reached a volume of 75-125 mm³. The groups were treated with vehicle, radiation, SCV-07 or radiation and SCV-07 as detailed in Table 2.1. Initiation of drug treatment was designated as day 1. Mice in groups 1 and 4 received vehicle by subcutaneous (sc) injection for 20 days. Mice in groups 2-4 and 6-8 received SCV-07 in vehicle once a day by sc injection on days 1 through 20, and mice in groups 6-8 received radiation (2 doses of 4 Gy/dose on days 0 and 2). Radiation was done by anesthetizing the mice in these groups with ketamine (120 mg/kg) and xylazine (6 mg/kg), and placing them under a lead shield such that the region of the flank with tumor was exposed to the radiation. Radiation was delivered using a Philips 160 kV source at a focal distance of approximately 40 cm, and a dose rate of approximately 1.0 Gy/min. Tumors were measured on alternating days throughout the duration of the study. Mice in groups 1-8 were sacrificed on day 21 and remaining tumors were excised, measured, weighed, photographed and fixed in formalin for later analysis.

TABLE 2.1
SCI-05: Study Design

| | Number | Tumor | RT | Drug | | | |
|---|---|---|---|---|---|---|---|
| | of | cell | Days 0 | Treatment | | | |
| Group | animals | inoculum | and 2 | & Dosing | Route | Frequency | Schedule |
| 1 | 12 | 1 × 10⁵ | none | Vehicle | sc | bid | Day 1 to 20 |
| 2 | 12 | 1 × 10⁵ | none | SCV-07 | sc | bid | Day 1 to 20 |
| | | | | 100 μg/kg | | | |
| 3 | 12 | 1 × 10⁵ | none | SCV-07 | sc | bid | Day 1 to 20 |
| | | | | 1.0 mg/kg | | | |
| 4 | 12 | 1 × 10⁵ | none | SCV-07 | sc | bid | Day 1 to 20 |
| | | | | 10.0 mg/kg | | | |
| 5 | 12 | 1 × 10⁵ | 4 Gy | Vehicle | sc | bid | Day 1 to 20 |
| | | | focal | | | | |
| 6 | 12 | 1 × 10⁵ | 4 Gy | SCV-07 | sc | bid | Day 1 to 20 |
| | | | focal | 100 μg/kg | | | |
| 7 | 12 | 1 × 10⁵ | 4 Gy | SCV-07 | sc | bid | Day 1 to 20 |
| | | | focal | 1.0 mg/kg | | | |
| 8 | 12 | 1 × 10⁵ | 4 Gy | SCV-07 | sc | bid | Day 1 to 20 |
| | | | focal | 10.0 mg/kg | | | |

### Weights and Survival

To assess possible toxicity, animals were weighed every day and their survival recorded. Any animals exhibiting a loss of >20% of starting weight during the course of the study were euthanized. Any animals whose tumors grew to over 4000 mm³ were also euthanized.

### Materials and Methods

### Tissue Culture.

H146 human lung cancer cells were obtained from ATCC. These cells were grown in DMEM supplemented with 10% Fetal Calf Serum (FCS), 1% penicillin and streptomycin, and 2 mM L-Glutamine. Cells were sub-cultured by removing the medium, rinsing twice with sterile calcium- and magnesium-free phosphate buffered saline (PBS) and adding 1 to 2 ml of 0.25% trypsin/0.03% EDTA solution. The flask was incubated at 37° C. until cells detached. Cells were then sub-cultured at a ratio of 1:3.

### Location of Study Performance

The study was performed at Biomodels AAALAC accredited facility in Watertown Mass. Animal use approval for this study was obtained from Biomodels IACUC.

### Animals

Female nude mice, homozygous for the nu gene (nu+/nu+) (Charles River Labs), aged 5 to 6 weeks, with a mean pre-treatment body weight of 24 grams were used. Animals were individually numbered using an ear punch, housed in groups of 6 animals per cage, and acclimatized prior to study commencement. During the acclimatization period of at least 2 days, the animals were observed daily in order to reject animals that presented in poor condition.

### Housing

The study was performed in animal rooms provided with filtered air at a temperature of 70° F.+/−5° F. and 50%+/−20% relative humidity. Animal rooms were set to maintain a minimum of 12 to 15 air changes per hour. The room was on an automatic timer for a light/dark cycle of 12 hours on and 12 hours off with no twilight.

Sterilized Bed-O-Cobs® bedding was used. Bedding was changed a minimum of once per week.

Cages, tops, bottles, etc. were washed with a commercial detergent and allowed to air dry. Prior to use, these items were wrapped and autoclaved. A commercial disinfectant was used to disinfect surfaces and materials introduced into the hood. Floors were swept daily and mopped a minimum of twice weekly with a commercial detergent. Walls and cage racks were sponged a minimum of once per month with a dilute bleach solution. A cage card or label with the appropriate information necessary to identify the study, dose, animal number and treatment group marked all cages. The temperature and relative humidity were recorded during the study, and the records retained.

### Diet

Animals were fed with sterile Labdiet® 5053 (pre-sterilized) rodent chow and sterile water was provided ad libitum.

### Animal Randomization and Allocations

Mice were randomly and prospectively divided into eight (8) groups prior to the initiation of treatment. Each animal was identified by ear punching corresponding to an individual number. A cage card was used to identify each cage and marked with the study number (SCI-05), treatment group number and animal numbers.

### Assessment of Results

Statistical differences between treatment groups were determined using Student's t-test, Mann-Whitney U test and chi-square analysis with a critical value of 0.05.

### Experimental Procedures

Tumors were measured once every two days with micro-calipers, and tumor volume was calculated as 4/3πr³, where r is equal to the sum of the length and the width divided by 4. The tumor growth index (TGI) was calculated using the formula 100-(Vc*100/Vt), where Vc is the mean volume of the tumors in the control group and Vt is the mean volume of the tumors in the test group.

### Results and Discussion

### Survival

No animal deaths occurred as a direct result of treatment during the course of this study.

### Animal Weights

There were no significant differences in mean daily weight changes between vehicle-treated groups and animals who received SCV-07 as a mono-therapy (p=0.7) or in animals who received radiation only and SCV-07 in conjunction with radiotherapy (p=0.68). The mice receiving vehicle only gained an average of 13.2% of their starting weight by Day 21. Mice treated with either 100 μg/kg, 1.0 mg/kg or 10 mg/kg SCV-07 gained between 10.2% and 12.3% of their starting weight by Day 21. Mice treated with vehicle and exposed to radiation gained an average on 3.2% of their starting weight by Day 21. Mice treated with either 100 μg/kg, 1.0 mg/kg or 10 mg/kg SCV-07 and were exposed to radiation gained between 2.8% and 3.6% of their starting weight by Day 21.

The significance of these differences was evaluated by calculating the mean area under the curve (AUC) for the percentage weight change for each animal and comparing the groups using a One-Way ANOVA test.

### Tumor Volumes

Tumor volumes were calculated from the length and width measurements taken on alternating days by calculating the mean radius (r), which was the sum of length and width divided by 4, and using the formula 4/3 πr³ to calculate the volume.

Tumors from animals treated with 100 μg/ml grew at rates faster than vehicle control animals. Among the non-irradiated animals, mice treated with 10 mg/kg of SCV-07 showed the best improvement in tumor growth inhibition. The mean tumor volume at the end of the study period for vehicle treated animals was 4436.6 mm², 4923 mm² for 100 μg/kg SCV-07 treated animals, 4033.4 mm² for 1 mg/kg SCV-07 treated animals, and 2842.4 mm² for 10 mg/kg SCV-07 treated animals.

Among the irradiated animals, mice treated with 10 mg/kg of SCV-07 showed the best improvement in tumor growth inhibition. The mean tumor volume at the end of the study period for vehicle treated animals was 1618.5 mm², 1322.3 mm² for 100 μg/kg SCV-07 treated animals, 1923.9 mm² for 1 mg/kg SCV-07 treated animals, and 962.8 mm² for 10 mg/kg SCV-07 treated animals.

Further analysis of the data was performed by calculating the mean area under the curve (AUC) for the tumor volume for each animal and comparing the groups using a One-Way ANOVA test. This analysis did not reveal significant differences between any of the treated groups and the saline control group (p=0.13 for the non-irradiated animals, and p=0.14 for irradiated animals). However, a direct comparison of vehicle treated and 10 mg/kg SCV-07 with a Mann-Whitney Rank Sum analysis was significantly different (p=0.026).

The tumor growth inhibition (TGI) was calculated using the formula 100-(Vc*100/Vt), where Vc is the mean volume of the tumors in the control group and Vt is the mean volume of the tumors in the test group. Table 2.2 shows the tumor growth inhibition for animals treated with 100 μg/kg, 1 mg/kg, 10 mg/kg SCV-07 alone or in combination with radiation. When compared to unirradiated controls, animals treated with 1 mg/kg SCV-07 alone had a tumor growth inhibition of 9.1%, and animals treated with 10 mg/kg SCV-07 alone had a tumor growth inhibition of 35.9%. Animals treated with radiation alone had a TGI of 63.5% when compared to unirradiated controls, while animals treated with SCV-07 plus radiation had TGI values of 70.2% (100 μg/kg), 50.3% (1 mg/kg) and 78.3% (10 mg/kg). When compared to the group receiving radiation plus vehicle the groups treated with radiation plus SCV-07 had a TGI of 18.3% at 100 μg/kg and 40.5% at 10 mg/kg.

TABLE 2.2
Table 2.2. Tumor Growth Inhibition (TGI).

| | | Drug | | Mean Tumor | | |
|---|---|---|---|---|---|---|
| | RT Days | Treatment | | Volume | | |
| Group | 0 and 2 | & Dosing | | (mm³) | TGI (%) | TGI (%) |
| 1 | — | Vehicle | | 4436.5 | — | |
| 2 | — | SCV-07 | | 4922.8 | ** | |
| | | 100 | μg/kg | | | |
| 3 | — | SCV-07 | | 4033.4 | 9.1 | |
| | | 1.0 | mg/kg | | | |
| | | 2.0 | | | | |
| 4 | — | SCV-07 | | 2842.4 | 35.9 | |
| | | 10.0 | mg/kg | | | |
| 5 | 4 Gy | Vehicle | | 1618.5 | 63.5 | — |
| | focal | | | | | |
| 6 | 4 Gy | SCV-07 | | 1322.3 | 70.2 | 18.3 |
| | focal | 100 | μg/kg | | | |
| 7 | 4 Gy | SCV-07 | | 1923.9 | 50.3 | ** |
| | focal | 1.0 | mg/kg | | | |
| | | 2.0 | | | | |
| 8 | 4 Gy | SCV-07 | | 962.8 | 78.3 | 40.5 |
| | focal | 10.0 | mg/kg | | | |

TGI was calculated from the last tumor measurement using the formula 100-(Vc*100/Vt), where Vc is the mean volume of the tumors in the contol group and Vt is the mean volume of the tumors in the test group.
**Mean tumor volumes in groups 2 and 7 exceeded the vehicle control animals (9.8% and 15.87%, respectively).

### Conclusions

SCV-07 showed no evidence of toxicity in this study based on observations of survival and weight change.

Animals treated with 10 mg/kg SCV-07 alone showed significant reduction in tumor growth inhibition (TGI=68%) compared to vehicle control animals (P=0.026).

Although not statistically significant, animals treated with 100 μg/kg SCV-07 alone or with 1 mg/kg SCV-07 alone showed reductions in tumor growth compared to animals who received vehicle only.

Although not statistically significant, irradiated animals treated with SCV-07 at 100 μg/kg or 10 mg/kg showed reductions in tumor growth relative to irradiated vehicle control animals.

APPENDIX 2.1.
Daily Animal Weight

| | | Weight | | | | | | | | | | | | | | | | | | | | |
|---|---|---|---|---|---|---|---|---|---|---|---|---|---|---|---|---|---|---|---|---|---|---|
| | | | Day | | Day | | Day | | | | Day | | | | Day | | | | Day | | | |
| Group | No. | Day 1 | 2 | Day 3 | 4 | Day 5 | 6 | Day 7 | Day 8 | Day 9 | 10 | Day 11 | Day 12 | Day 13 | 14 | Day 15 | Day 16 | Day 17 | 18 | Day 19 | Day 20 | Day 21 |
| 1 | 1 | 24.22 | 23.81 | 23.97 | 23.63 | 23.56 | 25.61 | 23.71 | 24.2 | 24.9 | 24.86 | 24.95 | 25.04 | 26.32 | 25.44 | 26.43 | 28.7 | 27.62 | | | | |
| 1 | 2 | 26.48 | 27.72 | 27.62 | 27.73 | 26.78 | 27.71 | 26.36 | 26.63 | 26.71 | 26.89 | 27.56 | 27.53 | 28.21 | 27.51 | 28.63 | 28.19 | 29.51 | | | | |
| 1 | 3 | 22.49 | 22.8 | 23 | 23.11 | 23.34 | 23.21 | 23.01 | 23.73 | 23.56 | 23.35 | 23.38 | 23.28 | 24.15 | 23.8 | 24.08 | 24.06 | 24.78 | 25.38 | 25.54 | 25.41 | 25.19 |
| 1 | 4 | 24.89 | 25.39 | 25.94 | 26.71 | 26.7 | 28.32 | 26.85 | 27.52 | 27.81 | 27.06 | 27.48 | 27.35 | 28.08 | 28.07 | 29.28 | 28.99 | 29.32 | | | | |
| 1 | 5 | 25.69 | 25.58 | 25.8 | 25.9 | 25.87 | 26.23 | 25.75 | 26.03 | 27.05 | 26.69 | 26.72 | 26.94 | 27.62 | 28.75 | 29.16 | 30.54 | 31.74 | | | | |
| 1 | 6 | 24.97 | 26.54 | 27.55 | 29.86 | 27.7 | 26.65 | 25.93 | 23.93 | 26.78 | 27.02 | 25.66 | 26.15 | 26.71 | 26.56 | 26.43 | 26.89 | 27.13 | 27.25 | 26.5 | 27.43 | 28.37 |
| 1 | 7 | 26.64 | 27.18 | 27.39 | 27.87 | 28.14 | 27.48 | 28.21 | 28.44 | 29.11 | 29.41 | 30.24 | 29.65 | 29.02 | 27.73 | 29.23 | 31.18 | 26.71 | | | | |
| 1 | 8 | 27.43 | 28.12 | 28.37 | 28.56 | 28.72 | 29.75 | 29.39 | 29.93 | 31.55 | 31.41 | 31.86 | 31.97 | 33.03 | 31.77 | 30.86 | 30.99 | 29.79 | | | | |
| 1 | 9 | 27.24 | 29.48 | 29.61 | 27.56 | 30.1 | 30.02 | 28.98 | 29.49 | 29.18 | 28.77 | 28.38 | 28.25 | 28.62 | 27.74 | 27.64 | 27.7 | 28.26 | | | | |
| 1 | 10 | 23.86 | 24.09 | 24.59 | 24.62 | 25.01 | 25.4 | 24.83 | 25.07 | 25.34 | 24.63 | 24.92 | 24.89 | 25.13 | 25.38 | 25.67 | 26.32 | 27.29 | | | | |
| 1 | 11 | 25.04 | 25.31 | 25.54 | 26.03 | 26.25 | 26.11 | 25.31 | 26.02 | 26.02 | 26.04 | 26.3 | 26.11 | 26.4 | 25.06 | 25.37 | 25.97 | 26.19 | 25.84 | 25.92 | 26.45 | 25.87 |
| 1 | 12 | 25.56 | 25.66 | 26.49 | 25.96 | 26.02 | 27.33 | 26.07 | 26.81 | 27.61 | 27.72 | 28.4 | 28.91 | 29.74 | 29.76 | 31.51 | 31.62 | 34.69 | | | | |
| 2 | 13 | 23.33 | 24.24 | 23.54 | 23.25 | 22.95 | 23.19 | 22.44 | 22.45 | 22.32 | 21.63 | 21.55 | 21.12 | 21.27 | 20.98 | 25.31 | 21.55 | 21.85 | 22.27 | 22.56 | 21.88 | 23.21 |
| 2 | 14 | 23.49 | 24.15 | 24.31 | 24.83 | 24.17 | 24.63 | 24.41 | 25.4 | 24.63 | 24.52 | 24.68 | 25.14 | 25.5 | 24.94 | 25.45 | 25.58 | 25.81 | 26.93 | 26.87 | 26.82 | 28.61 |
| 2 | 15 | 21.12 | 21.62 | 21.57 | 22.26 | 22.13 | 22.87 | 22.25 | 23.38 | 23.39 | 23.32 | 24.31 | 24.12 | 24.4 | 23.42 | 23.95 | 24.04 | 24.69 | | | | |
| 2 | 16 | 25.34 | 27.17 | 27.05 | 27.87 | 27.63 | 28.32 | 27.57 | 23.43 | 28.44 | 28.36 | 28.42 | 28.53 | 29.09 | 29.02 | 29.26 | 29.34 | 30.14 | | | | |
| 2 | 17 | 23.81 | 24.75 | 24.23 | 24.51 | 23.62 | 24.73 | 23.85 | 24.2 | 24.12 | 23.75 | 23.95 | 24.81 | 24.69 | 24.51 | 24.82 | 24.85 | 26.29 | | | | |
| 2 | 18 | 26.25 | 27.62 | 27.72 | 28.09 | 27.38 | 28.41 | 27.66 | 27.81 | 28.18 | 27.17 | 27.9 | 27.09 | 28.31 | 27.54 | 27.53 | 25.33 | 27.77 | | | | |
| 2 | 19 | 23.52 | 23.55 | 23.75 | 24.23 | 23.58 | 24.17 | 24.37 | 24.12 | 24.62 | 24.21 | 24.76 | 24.69 | 25.03 | 25.71 | 25.48 | 27 | 27.41 | | | | |
| 2 | 20 | 27.05 | 28.22 | 28.04 | 28.63 | 28.59 | 29.52 | 29.36 | 31.1 | 32.42 | 32.28 | 33.1 | 34.51 | 35.6 | 36.51 | 38.88 | 39.72 | 39.18 | | | | |
| 2 | 21 | 22.89 | 24.07 | 24.19 | 24.54 | 24.34 | 24.43 | 24.33 | 24.96 | 25.24 | 25.05 | 25.74 | 26.22 | 24.9 | 25.44 | 27.69 | 26.23 | 19.73 | 20.08 | 21.18 | 22.97 | 24.47 |
| 2 | 22 | 27.22 | 28.42 | 27.98 | 28.47 | 27.95 | 28.85 | 28.35 | 29.11 | 27.73 | 28.12 | 27.95 | 28.02 | 28.93 | 27.98 | 24.56 | 28.01 | 28.59 | 29.42 | 29.76 | 30.57 | 31.9 |
| 2 | 23 | 24.41 | 25.08 | 24.72 | 24.81 | 24.42 | 25.02 | 24.36 | 25.53 | 24.91 | 24.71 | 25.01 | 25.22 | 25.62 | 24.64 | 21.53 | 25.33 | 24.98 | 25.41 | 25.04 | 25.04 | 25.72 |
| 2 | 24 | 23.73 | 24.13 | 24.83 | 25.45 | 24.92 | 24.63 | 24.82 | 25.06 | 24.86 | 24.81 | 24.8 | 24.56 | 26.34 | 24.13 | 25.13 | 24.78 | 27.14 | | | | |
| 3 | 25 | 25.6 | 27.18 | 27.14 | 27.96 | 27.53 | 28.33 | 28.33 | 23.58 | 28.73 | 28.58 | 29.12 | 28.92 | 29.13 | 27.87 | 26.98 | 25.7 | 25.39 | | | | |
| 3 | 26 | 24.48 | 25.72 | 25.6 | 25.35 | 25.79 | 26.31 | 25.73 | 25.62 | 25.99 | 26.27 | 25.88 | 26.65 | 27.21 | 26.39 | 28.39 | 26.83 | 27.8 | | | | |
| 3 | 27 | 24.53 | 25.21 | 25.71 | 25.56 | 25.23 | 25.83 | 25.6 | 25.34 | 25.75 | 2.89 | 26.11 | 26.59 | 27.33 | 27.02 | 27.6 | 27.84 | 28.91 | | | | |
| 3 | 28 | 26.48 | 27.22 | 26.81 | 26.82 | 26.44 | 26.32 | 26.14 | 26.52 | 26.71 | 26.76 | 26.69 | 27.07 | 28.31 | 27.32 | 27.85 | 28.03 | 28.27 | 27.8 | 27.85 | 28.32 | 28.38 |
| 3 | 29 | 26.69 | 26.89 | 26.61 | 27.01 | 26.81 | 27.52 | 27.78 | 28.06 | 28.17 | 27.92 | 27.84 | 28.16 | 28.35 | 27.72 | 24.2 | 28.72 | 29.18 | 29.45 | 29.08 | 28.73 | 29 |
| 3 | 30 | 23.08 | 23.45 | 23.45 | 23.71 | 23.81 | 22.45 | 24.45 | 24.81 | 25.24 | 25.4 | 25.77 | 26.54 | 26.07 | 27.71 | 26.73 | 27.32 | 26.44 | | | | |
| 3 | 31 | 21.47 | 22.3 | 22.44 | 22.49 | 22.8 | 24.65 | 22.6 | 23.04 | 23.56 | 23.85 | 23.72 | 23.81 | 29.41 | 24.59 | 24.81 | 26.4 | 26.25 | 26.01 | 26.47 | 27.1 | 28.13 |
| 3 | 32 | 27.78 | 29.54 | 29.27 | 29.91 | 29.77 | 29.51 | 29.23 | 29.95 | 29.94 | 29.74 | 29.44 | 29.91 | 30.71 | 29.75 | 29.87 | 30.68 | 31.4 | 31.52 | 33.23 | 32.64 | 34.72 |
| 3 | 33 | 25.54 | 26.22 | 26.23 | 26.39 | 25.75 | 26.85 | 26.02 | 26.6 | 26.62 | 26.81 | 27.34 | 28.09 | 29.13 | 28.69 | 30.12 | 30.53 | 31.38 | | | | |
| 3 | 34 | 28.85 | 29.47 | 28.9 | 29.55 | 29.01 | 29.63 | 28.63 | 29.19 | 28.62 | 29.02 | 28.41 | 29.04 | 24.08 | 28.46 | 28.98 | 29.1 | 30.11 | | | | |
| 3 | 35 | 23.89 | 24.89 | 24.3 | 24.82 | 24.54 | 24.78 | 24.83 | 24.68 | 24.69 | 24.43 | 24.17 | 24.31 | 24.6 | 24.55 | 27.2 | 25.14 | 25.13 | | | | |
| 3 | 36 | 24.13 | 24.89 | 24.81 | 25.01 | 24.28 | 24.49 | 24.01 | 24.65 | 24.57 | 24.55 | 24.51 | 24.05 | 23.71 | 23.87 | 23.56 | 22.98 | 23.83 | | | | |
| 4 | 37 | 24.58 | 25.71 | 25.71 | 26.31 | 23.6 | 24.12 | 25.77 | 26.5 | 26.46 | 26.44 | 26.59 | 26.08 | 26.43 | 26.23 | 26.5 | 26.37 | 26.34 | | | | |
| 4 | 38 | 20.02 | 20.51 | 19.65 | 19.79 | 19.38 | 20.16 | 19.24 | 19.57 | 19.41 | 19.38 | 19.8 | 19.71 | 19.78 | 19.36 | 19.45 | 19.75 | 20.46 | 21.6 | 21.66 | 22.28 | 23.08 |
| 4 | 39 | 25.88 | 27.38 | 27.55 | 27.37 | 27.19 | 27.27 | 27.22 | 27.95 | 27.52 | 27.55 | 27.85 | 27.58 | 27.61 | 26.73 | 26.98 | 27.26 | 27.38 | | | | |
| 4 | 40 | 20.19 | 22.33 | 22.29 | 22.76 | 22.92 | 23.1 | 22.18 | 22.21 | 22.56 | 22.37 | 23.11 | 22.55 | 22.52 | 22.05 | 22.31 | 22.46 | 23.34 | 23.41 | 24.23 | 25.08 | 25.71 |
| 4 | 41 | 23.36 | 25.08 | 24.56 | 25.59 | 25.28 | 26 | 25.12 | 25.19 | 24.94 | 25.29 | 25.66 | 25.41 | 25.47 | 24.42 | 24.23 | 25.54 | 25.85 | 25.98 | 26.51 | 27.22 | 28.04 |
| 4 | 42 | 23.92 | 25.16 | 24.82 | 25.2 | 24.94 | 25.29 | 24.58 | 24.75 | 24.58 | 24.41 | 24.86 | 25.02 | 25.22 | 24.77 | 24.17 | 25.76 | 26.59 | 26.85 | 27.51 | 28.16 | 29.12 |
| 4 | 43 | 23.39 | 23.33 | 23.29 | 23.86 | 24.32 | 24.46 | 24.81 | 25.58 | 26.05 | 25.73 | 25.84 | 25.61 | 25.42 | 24.52 | 25.33 | 24.96 | 25.19 | | | | |
| 4 | 44 | 23.49 | 24.53 | 24.49 | 25.06 | 24.61 | 24.45 | 23.43 | 23.44 | 22.79 | 22.71 | 23.58 | 23.09 | 22.57 | 21.73 | 22.43 | 22.07 | 22.55 | 22.84 | 22.79 | 23.7 | 24.01 |
| 4 | 45 | 24.75 | 24.68 | 24.07 | 23.84 | 23.69 | 23.58 | 22.81 | 22.48 | 21.66 | 21.03 | 21.18 | 20.51 | 21.32 | 21.66 | 21.75 | 22.04 | 22.16 | 22.05 | 22.22 | 21.59 | 21.59 |
| 4 | 46 | 25.62 | 27.02 | 26.11 | 26.58 | 24.98 | 26.68 | 26.1 | 26.24 | 26.24 | 26.34 | 26.11 | 26.1 | 24.46 | 25.69 | 23.5 | 26.58 | 27.28 | | | | |
| 4 | 47 | 22.16 | 23.43 | 23.24 | 23.52 | 23.24 | 26.19 | 23.4 | 23.64 | 24.02 | 24.23 | 24.1 | 24.2 | 26.68 | 23.62 | 26.57 | 24.1 | 25.32 | | | | |
| 4 | 48 | 24.93 | 25.52 | 25.16 | 25.52 | 25.62 | 26.55 | 25.36 | 25.99 | 25.58 | 25.32 | 26.01 | 26.18 | 26.16 | 25.54 | 26.18 | 26.32 | 26.76 | | | | |

| | | Weight | | | | | | | | | | | | | | | | | | | | | |
|---|---|---|---|---|---|---|---|---|---|---|---|---|---|---|---|---|---|---|---|---|---|---|---|
| | | | Day | | Day | | Day | | Day | | Day | | Day | | Day | | Day | | Day | | Day | | |
| Group | No. | Day 0 | 1 | Day 2 | 3 | Day 4 | 5 | Day 6 | 7 | Day 8 | 9 | Day 10 | 11 | Day 12 | 13 | Day 14 | 15 | Day 16 | 17 | Day 18 | 19 | Day 20 | Day 21 |
| 5 | 49 | 25.45 | 23.69 | 23.64 | 22.77 | 22.99 | 22.34 | 23 | 24.43 | 23.46 | 22.09 | 22.27 | 22.51 | 22.81 | 23.92 | 23.93 | 23.5 | 23.96 | 24.25 | 23.71 | 23.84 | 24.04 | 23.88 |
| 5 | 50 | 22.89 | 21.55 | 21.6 | 21.13 | 21.46 | 21.29 | 21.97 | 22.44 | 22.14 | 21.96 | 22.24 | 22.03 | 21.99 | 22.84 | 22.9 | 22.56 | 22.85 | 22.26 | 23.37 | 22.72 | 23.1 | 23.69 |
| 5 | 51 | 22.49 | 21.91 | 22.47 | 21.39 | 21.72 | 21.36 | 22.23 | 22.52 | 21.94 | 22.09 | 22.29 | 21.85 | 22.18 | 22.21 | 23.01 | 22.75 | 22.59 | 22.49 | 22.77 | 22.34 | 22.71 | 23.38 |
| 5 | 52 | 24.85 | 23.65 | 23.98 | 21.15 | 24.35 | 24.05 | 25 | 23.17 | 24.61 | 24.96 | 23.46 | 25.26 | 25.44 | 25.9 | 26.42 | 26.25 | 27.7 | 27.02 | 27.14 | 27.6 | 28.89 | 28.4 |
| 5 | 53 | 24.48 | 23.51 | 23.84 | 22.35 | 23.47 | 23.63 | 24.1 | 24.5 | 24.36 | 23.71 | 24.9 | 23.27 | 23.47 | 23.95 | 24.22 | 23.9 | 23.83 | 24.12 | 24.58 | 24.2 | 24.24 | 24.38 |
| 5 | 54 | 22.5 | 21.97 | 21.72 | 23.02 | 21.72 | 22.27 | 22.82 | 22.81 | 22.48 | 21.74 | 22.04 | 22.15 | 21.96 | 22.48 | 23.62 | 22.83 | 23.06 | 22.01 | 23.18 | 22.49 | 23.29 | 23.49 |
| 5 | 55 | 22.92 | 22.02 | 22.58 | 23.35 | 21.87 | 21.2 | 22.66 | 24.63 | 22.56 | 22.81 | 22.6 | 22.36 | 21.98 | 22.27 | 22.91 | 22.27 | 22.26 | 22.64 | 22.54 | 22.68 | 22.83 | 22.59 |
| 5 | 56 | 22.11 | 21.65 | 22.34 | 21.84 | 21.6 | 21.65 | 21.7 | 22.44 | 22.25 | 22.12 | 21.93 | 22.14 | 22.05 | 22.85 | 23.44 | 23.4 | 23.3 | 23.17 | 24.02 | 23.94 | 23.31 | 23.62 |
| 5 | 57 | 24.7 | 23.64 | 24.03 | 23.52 | 23.11 | 23.34 | 24.09 | 23.91 | 23.46 | 23.5 | 23.91 | 23.4 | 23.52 | 23.31 | 24.24 | 23.8 | 24.34 | 24.01 | 24.64 | 23.95 | 24.14 | 24.31 |
| 5 | 58 | 23.36 | 22.3 | 22.63 | 23.39 | 22.28 | 22.29 | 22.91 | 23.18 | 23.19 | 27.74 | 22.6 | 22.37 | 22.72 | 22.98 | 23.82 | 24.05 | 24.13 | 23.72 | 24.07 | 23.32 | 23.98 | 24.17 |
| 5 | 59 | 20.78 | 20.82 | 21.29 | 21.95 | 21.08 | 21.18 | 22.18 | 22.5 | 22.69 | 21.6 | 22.4 | 22.36 | 22.29 | 22.5 | 22.63 | 22.72 | 22.34 | 22.29 | 22.92 | 22.54 | 22.85 | 22.9 |
| 5 | 60 | 23.92 | 22.52 | 22.39 | 21.95 | 22.16 | 21.74 | 23.12 | 23.14 | 23.47 | 23.09 | 23.83 | 24.33 | 24.26 | 24.64 | 25.61 | 25.34 | 25.31 | 24.49 | 25.32 | 24.44 | 24.81 | 24.3 |
| 6 | 61 | 22.89 | 21.71 | 25.97 | 21.95 | 22.16 | 22.13 | 24.02 | 23.7 | 25.46 | 23.56 | 22.12 | 22.95 | 23.05 | 23.1 | 23.91 | 23.04 | 23.36 | 23.07 | 26.33 | 23.31 | 23.35 | 22.89 |
| 6 | 62 | 24.62 | 23.09 | 24.21 | 23.47 | 22.91 | 23.05 | 25.92 | 23.91 | 23.19 | 23.21 | 22.07 | 22.86 | 23.1 | 23.12 | 23.82 | 23.59 | 23.85 | 23.87 | 24.11 | 23.92 | 24.69 | 24.49 |
| 6 | 63 | 23.65 | 23.25 | 22.8 | 21.63 | 21.92 | 21.2 | 23.09 | 23.1 | 22.96 | 22.65 | 22.83 | 23.05 | 23.41 | 23.35 | 24.42 | 24.02 | 24.3 | 23.95 | 25.3 | 24.55 | 24.5 | 24.5 |
| 6 | 64 | 24.93 | 24.12 | 24.3 | 24.45 | 23.21 | 23.62 | 24.57 | 25.81 | 25.21 | 25.2 | 24.98 | 25.12 | 25.3 | 25.96 | 27 | 26.72 | 27.14 | 26.99 | 28.62 | 28.12 | 28.4 | 28.41 |
| 6 | 65 | 23.84 | 22.14 | 23.43 | 22.79 | 22.89 | 22.42 | 24.34 | 24 | 24.33 | 23.84 | 23.86 | 23.95 | 24.28 | 24.28 | 24.35 | 24.12 | 24.25 | 23.44 | 24.36 | 24.17 | 24.89 | 24.72 |
| 6 | 66 | 26.09 | 25.53 | 25.98 | 26.26 | 26.19 | 25.6 | 26.74 | 26.36 | 24.52 | 25.47 | 25.72 | 25.4 | 25.34 | 25.84 | 26.7 | 25.82 | 25.82 | 25.86 | 24.7 | 26.38 | 28.39 | 26.28 |
| 6 | 67 | 23.31 | 22 | 22.91 | 22.7 | 23.01 | 23.14 | 23.95 | 23.8 | 23.98 | 23.56 | 23.62 | 23.77 | 23.68 | 23.84 | 24.67 | 23.71 | 24.02 | 21.74 | 22.02 | 23.92 | 24.14 | 24.47 |
| 6 | 68 | 22.97 | 21.12 | 23.22 | 22.21 | 22.45 | 22.42 | 24.25 | 24.5 | 24.08 | 24.2 | 24.34 | 24.07 | 24.18 | 24.19 | 24.52 | 24.09 | 24.06 | 23.41 | 24.03 | 23.56 | 23.77 | 24.13 |
| 6 | 69 | 23.84 | 22.63 | 23.68 | 22.79 | 22.05 | 22.4 | 23.89 | 23.62 | 23.89 | 22.64 | 23.69 | 23.51 | 23.6 | 23.52 | 23.56 | 23.25 | 23.41 | 23.15 | 23.53 | 23.24 | 23.29 | 23.05 |
| 6 | 70 | 23.82 | 22.31 | 22.74 | 22.18 | 21.1 | 21.99 | 22.23 | 22.49 | 24.04 | 21.86 | 23.05 | 21.74 | 22.19 | 21.91 | 22.88 | 22.37 | 22.62 | 22.74 | 23.6 | 22.98 | 23.17 | 23.32 |
| 6 | 71 | 26.44 | 24.89 | 22.06 | 25.35 | 25.13 | 24.83 | 25.75 | 26 | 22.15 | 25.49 | 25.65 | 25.43 | 25.42 | 25.84 | 26.4 | 25.92 | 26.09 | 25.37 | 25.42 | 25.86 | 25.47 | 26.12 |
| 6 | 72 | 21.95 | 22.08 | 22.2 | 22.22 | 22.3 | 21.79 | 22.65 | 22.58 | 23.05 | 22.61 | 22.69 | 22.95 | 23.09 | 22.96 | 23.62 | 23.01 | 23.3 | 23.14 | 23.9 | 23.27 | 23.71 | 23.99 |
| 7 | 73 | 22.83 | 22.26 | 22.32 | 22.78 | 22.8 | 22.95 | 23.76 | 25.4 | 23.21 | 22.92 | 24.16 | 23.14 | 23.28 | 22.7 | 23.61 | 23.01 | 23.68 | 22.9 | 23.58 | 22.76 | 23.57 | 23.38 |
| 7 | 74 | 28.37 | 28.09 | 28.17 | 27.24 | 27.65 | 26.79 | 28.34 | 27.5 | 21.65 | 28.06 | 28.21 | 28.34 | 28.69 | 29.35 | 30.05 | 29.92 | 30.36 | 29.74 | 30.97 | 30.28 | 30.88 | 30.92 |
| 7 | 75 | 24.41 | 24.1 | 25.07 | 24.22 | 24.66 | 23.59 | 25.14 | 25.06 | 24.48 | 24.17 | 23.19 | 24.9 | 25.28 | 25.08 | 25.3 | 25.04 | 25.57 | 25.48 | 26.88 | 26.73 | 27.24 | 26.61 |
| 7 | 76 | 21.98 | 21.97 | 22.43 | 21.12 | 21.73 | 21.43 | 22.72 | 22.83 | 25.59 | 22.67 | 22.72 | 22.06 | 23.54 | 22.78 | 23.81 | 25.21 | 25.12 | 24.35 | 25.3 | 23.58 | 25.41 | 24.83 |
| 7 | 77 | 26.33 | 25.4 | 25.08 | 25.22 | 24.83 | 24.4 | 26 | 26.19 | 25.39 | 25.62 | 21.48 | 26.1 | 26.43 | 25.71 | 26.99 | 26.69 | 26.69 | 26.26 | 26.28 | 25.84 | 26.66 | 26.49 |
| 7 | 78 | 25.94 | 25.13 | 25.6 | 24.85 | 24.27 | 23.71 | 24.63 | 25.18 | 24.93 | 24.82 | 25.22 | 25.25 | 25.56 | 25.83 | 27.3 | 27.12 | 27.53 | 27.62 | 28 | 28.37 | 29.64 | 30 |
| 7 | 79 | 25.68 | 23.06 | 23.34 | 22.79 | 23.14 | 22.89 | 24.14 | 24.71 | 23.65 | 23.74 | 28.82 | 24.55 | 24.72 | 24.83 | 25.53 | 23.22 | 23.63 | 22.96 | 24.21 | 24.13 | 25.01 | 25.3 |
| 7 | 80 | 25.4 | 22.58 | 22.97 | 22.8 | 22.98 | 22.23 | 23.44 | 23.15 | 22.69 | 22.35 | 22.58 | 22.54 | 22.8 | 22.93 | 23.51 | 23.04 | 23.42 | 23.11 | 24.06 | 23.26 | 24.04 | 24.43 |
| 7 | 81 | 23 | 22.7 | 22.97 | 22.7 | 23.73 | 23.51 | 24.73 | 25.38 | 24.1 | 24.28 | 24.56 | 24.18 | 24.22 | 24.12 | 24.77 | 24.26 | 24.45 | 23.58 | 24.66 | 23.28 | 24.33 | 23.67 |
| 7 | 82 | 25.84 | 22.8 | 23.04 | 22.92 | 22.45 | 22.39 | 23.47 | 23.84 | 23.58 | 23.74 | 23.4 | 23.83 | 23.92 | 23.94 | 24.6 | 24.48 | 25.02 | 24.62 | 25.5 | 25.12 | 25.77 | 26.98 |
| 7 | 83 | 24.81 | 23.44 | 23.52 | 23.88 | 23.27 | 23.05 | 24.04 | 24.06 | 23.54 | 23.11 | 2.1 | 22.92 | 22.92 | 23.26 | 24.15 | 23.41 | 23.73 | 23.42 | 23.4 | 23.26 | 23.89 | 23.48 |
| 7 | 84 | 22.27 | 21.52 | 22.55 | 21.15 | 21.58 | 21.03 | 22.1 | 22.12 | 27.54 | 21.5 | 23.96 | 21.26 | 21.19 | 21.24 | 21.64 | 20.83 | 20.81 | 20.81 | 21.73 | 21.15 | 21.44 | 21.59 |
| 8 | 85 | 20.9 | 20.3 | 20.66 | 20 | 26.98 | 21.13 | 21.2 | 21.34 | 21.13 | 21.07 | 24.83 | 21.43 | 21.42 | 21.55 | 21.86 | 21.25 | 21.48 | 21.21 | 21.63 | 20.67 | 21.22 | 21.14 |
| 8 | 86 | 24.4 | 24.05 | 24.64 | 24.33 | 24.59 | 24.49 | 24.42 | 24.55 | 24.53 | 24.78 | 24.21 | 22.94 | 24.99 | 24.78 | 25.47 | 24.65 | 24.92 | 24.85 | 25.25 | 23.82 | 24.6 | 24.4 |
| 8 | 87 | 23.39 | 22.8 | 22.18 | 22.23 | 22.24 | 21.56 | 23.84 | 23.26 | 22.42 | 22.39 | 25.01 | 23.52 | 23.27 | 23.29 | 24.39 | 23.58 | 23.62 | 23.44 | 24.28 | 23.31 | 23.62 | 23.38 |
| 8 | 88 | 24.43 | 23.33 | 23.76 | 22.92 | 23.42 | 23.44 | 24.67 | 25 | 23.87 | 23.48 | 24.73 | 23.7 | 23.96 | 24.1 | 24 | 23.48 | 23.85 | 23.71 | 23.54 | 23.68 | 24.15 | 23.94 |
| 8 | 89 | 24.05 | 23.35 | 23.78 | 23.45 | 24.65 | 24.16 | 25.63 | 25.26 | 24.19 | 24.41 | 24.62 | 24.73 | 24.96 | 24.54 | 25.61 | 25.07 | 25.59 | 25.42 | 25.98 | 25.74 | 26.05 | 26.01 |
| 8 | 90 | 23.52 | 22.27 | 23.37 | 22.53 | 23.45 | 24.26 | 24.66 | 24.58 | 24.52 | 24.76 | 21.15 | 24.57 | 24.8 | 24.87 | 24.13 | 25.02 | 25.49 | 24.62 | 25.23 | 25.14 | 25.97 | 26.08 |
| 8 | 91 | 20.28 | 19.98 | 22.79 | 20.98 | 20.46 | 20.32 | 20.87 | 20.8 | 20.45 | 23.6 | 21.7 | 21.49 | 21.66 | 23.7 | 22.67 | 22.05 | 22.3 | 22.13 | 22.33 | 21.76 | 22.16 | 22.13 |
| 8 | 92 | 24.88 | 24.09 | 24.75 | 24.12 | 25.01 | 24.12 | 25.41 | 25.6 | 25.19 | 24.83 | 23.38 | 25.45 | 25.79 | 25.77 | 26.68 | 25.97 | 25.91 | 25.87 | 25.63 | 25.31 | 25.37 | 25.44 |
| 8 | 93 | 25.69 | 24.97 | 25.07 | 24.28 | 24.77 | 23.02 | 23 | 22.6 | 22.53 | 22.94 | 23.96 | 24.51 | 24.14 | 23.96 | 23.65 | 24.89 | 25.59 | 25.1 | 26.14 | 25.55 | 26.35 | 27.12 |
| 8 | 94 | 23.98 | 23.13 | 23.35 | 22.98 | 22.9 | 22.54 | 23.33 | 24.9 | 23.89 | 20.91 | 25.51 | 21.43 | 23.87 | 23.44 | 23.92 | 23.12 | 23.42 | 23.17 | 23.93 | 23.27 | 24.25 | 24.42 |
| 8 | 95 | 23.66 | 23.6 | 23.22 | 22.92 | 22.6 | 22.27 | 21.5 | 23.8 | sac | | | | | | | | | | | | | |
| 8 | 96 | 23.71 | 23.14 | 21.22 | 23.25 | 23.79 | 23.54 | 24.61 | 24.41 | 24.18 | 23.72 | 23.72 | 23.63 | 23.69 | 24.99 | 25.81 | 25.44 | 23.69 | 23.22 | 23.72 | 22.86 | 23.38 | 23.42 |

APPENDIX 2.2.
Tumor Dimensions

| | | Day 1 | | Day 3 | | Day 5 | | Day 7 | | Day 9 | | Day 11 | | Day 13 | | Day 15 | | Day 17 | | Day 19 | | Day 21 | |
|---|---|---|---|---|---|---|---|---|---|---|---|---|---|---|---|---|---|---|---|---|---|---|---|
| | | | Dim | | Dim | | Dim | | Dim | | Dim | | Dim | | Dim | | Dim | | Dim | | Dim | | Dim |
| Group | | Dim 1 | 2 | Dim 1 | 2 | Dim 1 | 2 | Dim 1 | 2 | Dim 1 | 2 | Dim 1 | 2 | Dim 1 | 2 | Dim 1 | 2 | Dim 1 | 2 | Dim 1 | 2 | Dim 1 | 2 |
| | No. | | | | | | | | | | | | | | | | | | | | | | |
| 1 | 1 | 11.23 | 6.75 | 13.44 | 8.83 | 14.47 | 9.6 | 14.02 | 10.75 | 9.85 | 14.29 | 16.52 | 14.44 | 21.32 | 14.99 | 21.35 | 14.75 | 20.58 | 16.71 | | | | |
| 1 | 2 | 6.51 | 4.01 | 7.53 | 5.82 | 6.77 | 8.31 | 8.16 | 9.9 | 10.12 | 10.96 | 13.53 | 11.71 | 14.06 | 12.14 | 16.09 | 15.87 | 16.35 | 18.98 | | | | |
| 1 | 3 | 4.77 | 7.57 | 8.29 | 5.57 | 6.24 | 9.06 | 8.36 | 6.27 | 6.73 | 7.77 | 12.71 | 8.85 | 13.45 | 8.38 | 14.28 | 11.05 | 17.99 | 13.4 | 13.85 | 19.42 | 22.3 | 17.45 |
| 1 | 4 | 8.27 | 6.22 | 9.8 | 9.1 | 11.04 | 9.94 | 77.67 | 12.07 | 12.55 | 11.35 | 18.17 | 14.51 | 19.13 | 15.99 | 21.29 | 19.38 | 19.76 | 23.73 | | | | |
| 1 | 5 | 8.16 | 10.21 | 11.38 | 8.81 | 13.41 | 9.61 | 10.29 | 17.33 | 16.09 | 10.91 | 15.96 | 13.5 | 17.45 | 14.31 | 15.75 | 17.07 | 12.92 | 17.4 | | | | |
| 1 | 6 | 3.46 | 3.67 | 7.92 | 4.89 | 7.05 | 4.59 | 6.72 | 6.44 | 7.48 | 8.13 | 12.38 | 9.66 | 13.63 | 10.62 | 15.14 | 14.67 | 16.49 | 15.61 | 17.74 | 18.2 | 20.47 | 18.67 |
| 1 | 7 | 13.81 | 10.99 | 17.4 | 13.99 | 15.29 | 20.56 | 21.5 | 17.35 | 22.19 | 18.02 | 25.16 | 21.95 | 24.02 | 22.38 | 18.47 | 22.3 | 23.66 | 20.94 | | | | |
| 1 | 8 | 11.45 | 10.85 | 14.07 | 12.06 | 15.37 | 13.72 | 15.13 | 18.73 | 20.43 | 18.34 | 23.77 | 21.23 | 26.22 | 22.3 | 26.22 | 23.8 | 18.27 | 20.22 | | | | |
| 1 | 9 | 6.97 | 5.1 | 9.81 | 6.33 | 8.58 | 6.79 | 991 | 8.11 | 8.35 | 8.74 | 13.59 | 10.9 | 13.21 | 12.03 | 12.23 | 13.67 | 16.11 | 16.94 | | | | |
| 1 | 10 | 12.56 | 7.66 | 13.52 | 10.15 | 15.22 | 10.4 | 14.35 | 11.46 | 16.27 | 10.62 | 19.28 | 13.71 | 19.5 | 14.35 | 14.96 | 21.79 | 24.58 | 16.15 | | | | |
| 1 | 11 | 8.19 | 7.19 | 6.74 | 5.01 | 6.9 | 4.88 | 5.84 | 4.41 | 3.04 | 4.11 | 6.95 | 4.97 | 6.29 | 4.77 | 6.75 | 7.06 | 7.61 | 7.54 | 8.53 | 7.75 | 10.73 | 9.41 |
| 1 | 12 | 8.89 | 8.57 | 12.78 | 10.4 | 15.82 | 13.3 | 15.55 | 14.08 | 15.47 | 15.91 | 25.78 | 21.27 | 27.33 | 24.56 | 27.39 | 24.56 | 28.2 | 32.78 | | | | |
| 2 | 13 | 4.84 | 4.53 | 5.08 | 4.06 | 7.53 | 5.59 | 8.12 | 5.97 | 5.25 | 7.18 | 10.04 | 6.99 | 11.2 | 7.32 | 10.84 | 7.86 | 9.76 | 14.5 | 15.2 | 11.81 | 11.65 | 17.98 |
| 2 | 14 | 3.54 | 5.66 | 6.88 | 5.36 | 6.63 | 6.29 | 7.92 | 6.6 | 8.96 | 6.86 | 13.55 | 10.75 | 12.85 | 10.32 | 10.32 | 12.45 | 12.17 | 18 | 17.11 | 19.26 | 23.23 | 15.62 |
| 2 | 15 | 6.42 | 8.48 | 8.34 | 7.4 | 8.24 | 8.73 | 10.19 | 10.57 | 9.69 | 12.41 | 17.02 | 13.82 | 16.14 | 15.71 | 15.79 | 16.01 | 18.84 | 20.51 | | | | |
| 2 | 16 | 6.89 | 7.29 | 8.77 | 8.13 | 11.89 | 9.32 | 14.72 | 10.48 | 13.17 | 11.54 | 22.2 | 16.26 | 24.46 | 16.15 | 21.06 | 16.57 | 21.33 | 19.04 | | | | |
| 2 | 17 | 8.38 | 6.42 | 9.6 | 7.75 | 9.32 | 10.5 | 10.45 | 9.54 | 12.71 | 10.67 | 15.68 | 13.61 | 17.05 | 13.82 | 16.79 | 17.23 | 17.77 | 18.64 | | | | |
| 2 | 18 | 4.05 | 4.15 | 7.29 | 4.45 | 6.98 | 5.28 | 5.6 | 8.47 | 8.34 | 7.1 | 12.8 | 9.45 | 14.32 | 9.81 | 14.88 | 14.31 | 13.15 | 16.82 | | | | |
| 2 | 19 | 10.05 | 6.81 | 13.57 | 8.24 | 14.72 | 9.63 | 10.53 | 18.09 | 12.48 | 19.39 | 25.23 | 17.05 | 25.51 | 18.23 | 18.25 | 25.51 | 22.54 | 30.53 | | | | |
| 2 | 20 | 12.12 | 12.56 | 13.9 | 11.89 | 18.42 | 12.53 | 16.3 | 20.47 | 20.7 | 16.15 | 26.53 | 19.84 | 26.76 | 24.31 | 32.03 | 31.56 | 33.33 | 29.77 | | | | |
| 2 | 21 | 10.25 | 8.18 | 13.2 | 8.91 | 13.91 | 9.32 | 9.65 | 14.5 | 10.76 | 15.12 | 19.82 | 16.32 | 13.18 | 10.73 | 10.37 | 13.46 | 16.79 | 14.4 | 17.8 | 19.51 | 18.08 | 21.6 |
| 2 | 22 | 4.77 | 5.84 | 7.02 | 6.41 | 7.24 | 6.5 | 6.86 | 7.3 | 6.13 | 5.94 | 11.01 | 9.3 | 11.85 | 9.77 | 21.41 | 17.2 | 12.74 | 16.81 | 22.26 | 20.76 | 23.51 | 18.06 |
| 2 | 23 | 5.99 | 4.81 | 6.89 | 4.51 | 5.03 | 6.28 | 4.24 | 6.5 | 4.39 | 6.24 | 8.54 | 7.08 | 8.94 | 6.24 | 6.26 | 8.57 | 8.8 | 11.28 | 11.93 | 11.45 | 9.68 | 10.99 |
| 2 | 24 | 5.76 | 4.2 | 5.96 | 4.57 | 5.54 | 4.42 | 6.88 | 6.48 | 6.14 | 6.01 | 10.64 | 8.98 | 20.32 | 16.24 | 20.23 | 16.57 | 21.3 | 20.29 | | | | |
| 3 | 25 | 9.69 | 8.01 | 12.16 | 10.13 | 14.51 | 11.34 | 18.26 | 14.58 | 19.51 | 16.81 | 22.36 | 19.62 | 23.61 | 20.15 | 14.4 | 15.98 | 18.93 | 18.17 | | | | |
| 3 | 26 | 5.53 | 3.9 | 7.45 | 6.39 | 7.41 | 8.2 | 7.97 | 9.75 | 9.39 | 10.2 | 14.86 | 12.62 | 15.64 | 13.6 | 12.94 | 16.9 | 17.15 | 20.18 | | | | |
| 3 | 27 | 6.17 | 6.66 | 8.78 | 6.96 | 9.82 | 7.75 | 6.87 | 12.04 | 9.43 | 11.69 | 16.35 | 11.46 | 17.6 | 13.56 | 19.41 | 17.26 | 19.18 | 23.35 | | | | |
| 3 | 28 | 6.74 | 3.05 | 9.08 | 5.31 | 8.28 | 4.4 | 5.58 | 8.91 | 5.62 | 8.06 | 11.61 | 7.59 | 11.74 | 7.05 | 12.99 | 8.78 | 14.08 | 9.5 | 11.06 | 12.96 | 15.08 | 13.54 |
| 3 | 29 | 7.15 | 8.85 | 9.65 | 7.53 | 8.09 | 8.23 | 9.18 | 6.63 | 9.76 | 7.41 | 14.25 | 14.32 | 13.21 | 11.2 | 13.23 | 13.41 | 15.05 | 13.89 | 16.57 | 17.01 | 17.98 | 15.95 |
| 3 | 30 | 11.72 | 9.67 | 20.48 | 11.1 | 21.87 | 10.99 | 11.71 | 22.31 | 12.86 | 21.75 | 28.05 | 16.69 | 30.33 | 16.65 | 30.61 | 23.09 | 18.18 | 29.13 | | | | |
| 3 | 31 | 6.98 | 9 | 10.58 | 7.52 | 13.18 | 7.81 | 11.11 | 9.85 | 11.3 | 11.29 | 15.86 | 11.71 | 13.73 | 10.5 | 21.06 | 15.59 | 16.56 | 20.96 | 19.42 | 22.48 | 18.14 | 24.31 |
| 3 | 32 | 3.62 | 3.69 | 5.36 | 4.4 | 5.43 | 4.77 | 5.62 | 6.73 | 6.76 | 7.29 | 11.7 | 9.53 | 12.07 | 8.72 | 13.55 | 14.5 | 17.04 | 17.67 | 21.72 | 23.21 | 20.87 | 24.54 |
| 3 | 33 | 9.12 | 6.29 | 11.14 | 8.03 | 12.06 | 7.9 | 14.85 | 10.38 | 10.03 | 15.54 | 22.54 | 13.52 | 23.17 | 14.48 | 23.12 | 19.47 | 19.81 | 26.95 | | | | |
| 3 | 34 | 4.55 | 6.01 | 7.04 | 4.84 | 6.23 | 5.22 | 6.51 | 7.47 | 7.14 | 7.64 | 11.31 | 10.08 | 19.33 | 14.65 | 12.13 | 15.14 | 12.96 | 15.14 | | | | |
| 3 | 35 | 7.14 | 6.32 | 7.8 | 5.36 | 7.77 | 5.16 | 8.42 | 6.01 | 7.12 | 8.04 | 11.4 | 9.68 | 12.78 | 10.49 | 16.96 | 19.19 | 15.96 | 12.68 | | | | |
| 3 | 36 | 10.31 | 11.39 | 14.47 | 14.06 | 13.03 | 12.76 | 15.83 | 14.87 | 16.31 | 24.69 | 21.6 | 19.63 | 21.43 | 20.15 | 19.31 | 19.98 | 20.77 | 20.99 | | | | |
| 4 | 37 | 9.04 | 7.21 | 12.55 | 8.46 | 13.59 | 10.41 | 10.01 | 15.32 | 14.14 | 13.4 | 18.11 | 15.42 | 19.86 | 15.86 | 20.45 | 18.74 | 23.04 | 19.09 | | | | |
| 4 | 38 | 7.09 | 5.5 | 8.32 | 5.72 | 7.12 | 4.91 | 6.23 | 8.1 | 8.39 | 7.39 | 11.37 | 9.07 | 11.11 | 10.18 | 11.18 | 13.29 | 12.74 | 13.63 | 18.16 | 17.13 | 23.01 | 19.45 |
| 4 | 39 | 5.85 | 7.98 | 9.65 | 6.35 | 6.59 | 7.45 | 8.25 | 10.87 | 11.42 | 9 | 15.86 | 10.8 | 14.8 | 9.05 | 16.45 | 13.87 | 11.99 | 18.66 | | | | |
| 4 | 40 | 6.12 | 5.22 | 9.09 | 6.36 | 7.67 | 7.8 | 6.5 | 9.14 | 5.62 | 7.17 | 10.87 | 8.75 | 14.35 | 8.41 | 14.28 | 8.45 | 18.59 | 10.26 | 13.49 | 17.11 | 22.54 | 14.21 |
| 4 | 41 | 7.38 | 4.89 | 8.72 | 6.19 | 7.06 | 6.86 | 7.32 | 9.37 | 6.99 | 8.22 | 13.65 | 10.31 | 14.74 | 9.73 | 13.23 | 13.2 | 14.22 | 16.73 | 13.8 | 16.96 | 19.71 | 16.43 |
| 4 | 42 | 5.97 | 7.08 | 8.86 | 6.5 | 8.9 | 10.25 | 9.34 | 8.63 | 9.02 | 11.35 | 14.75 | 13.85 | 16.48 | 15.74 | 19.32 | 17.88 | 14.88 | 20.38 | 20.04 | 18.81 | 22.22 | 24.5 |
| 4 | 43 | 10.34 | 8.19 | 12.71 | 9.88 | 10.54 | 12.11 | 11 | 9.47 | 11.69 | 10.5 | 13.68 | 11.98 | 13.49 | 11.45 | 12.88 | 11.79 | 13.69 | 13.5 | | | | |
| 4 | 44 | 4.47 | 3.96 | 5.33 | 4.7 | 5.16 | 5.3 | 5.02 | 5.57 | 4.75 | 5.31 | 8.11 | 6.52 | 8.62 | 6.81 | 7.85 | 5.06 | 11.55 | 12.82 | 12.04 | 10.46 | 16.93 | 12.66 |
| 4 | 45 | 7.86 | 3.96 | 7.64 | 4.56 | 6.32 | 4.64 | 3.96 | 7.24 | 2.9 | 6.06 | 9.27 | 4.96 | 8.65 | 3.92 | 8.42 | 7.96 | 6.19 | 7.81 | 9.51 | 7.18 | 8.29 | 9.58 |
| 4 | 46 | 4.5 | 5.66 | 7.46 | 5.54 | 7.11 | 6.5 | 6.18 | 8.11 | 7.82 | 6.55 | 12 | 9.93 | 14.6 | 15.54 | 12.63 | 11.89 | 16.77 | 14.28 | | | | |
| 4 | 47 | 7.28 | 6.59 | 9.49 | 8.19 | 9.39 | 10.33 | 12.59 | 12.94 | 13.19 | 13.46 | 17.15 | 15.96 | 13.45 | 12.32 | 14.45 | 14.12 | 15.7 | 13.55 | | | | |
| 4 | 48 | 9.23 | 8.72 | 10.64 | 10.48 | 9.93 | 11.81 | 11.32 | 12.52 | 11.93 | 12.38 | 16.47 | 14.18 | 16.54 | 15.08 | 16.45 | 14.87 | 15.73 | 17.1 | | | | |
| | Animal | | | | | | | | | | | | | | | | | | | | | | |
| 5 | 49 | 9.49 | 9.71 | 9.18 | 10.31 | 9.7 | 9.41 | 9.52 | 10.04 | 10.18 | 10 | 10.87 | 9.64 | 12.09 | 10.93 | 11.97 | 12.13 | 11.47 | 10.65 | 12.97 | 11.22 | 12.57 | 13 |
| 5 | 50 | 7.47 | 6.33 | 6.75 | 7.19 | 9.51 | 7.36 | 10.47 | 8.65 | 11.33 | 8.95 | 11.86 | 9.53 | 13.1 | 10.91 | 14.74 | 11.75 | 10.98 | 14.4 | 14.31 | 16.83 | 18.2 | 15.15 |
| 5 | 51 | 7.23 | 5.98 | 7.35 | 7.88 | 7.93 | 8.95 | 6.73 | 9.57 | 10.26 | 8.81 | 9.63 | 8 | 10.85 | 9.53 | 10.49 | 9.23 | 7.76 | 9.69 | 9.76 | 12.34 | 12.61 | 10.82 |
| 5 | 52 | 7.09 | 5.57 | 5.07 | 5.54 | 7.05 | 6.17 | 7.53 | 6.12 | 9.92 | 8.31 | 11.56 | 8.32 | 10.79 | 10.01 | 16.71 | 10.98 | 14.97 | 8.84 | 15.34 | 21.77 | 24.95 | 18.24 |
| 5 | 53 | 9.83 | 6.12 | 7.65 | 6.4 | 6.31 | 10 | 9.58 | 6.5 | 12.39 | 7.43 | 12.35 | 7.05 | 14.12 | 9.41 | 15.2 | 10.08 | 9.46 | 14.6 | 10.58 | 16.77 | 18.06 | 12.73 |
| 5 | 54 | 4.08 | 4.89 | 4.78 | 5.1 | 4.28 | 4.09 | 4.64 | 4.65 | 5.62 | 5.37 | 5.92 | 4.76 | 7.75 | 7 | 8.1 | 6.92 | 7.05 | 6.53 | 9.12 | 8.71 | 10.77 | 9.41 |
| 5 | 55 | 7.36 | 6.04 | 6.49 | 6.27 | 7.3 | 8.33 | 8.09 | 9.66 | 9.34 | 8.36 | 9.17 | 7.74 | 11.17 | 9.58 | 12.17 | 10.03 | 9.56 | 10.54 | 11.13 | 11.85 | 14.17 | 12.35 |
| 5 | 56 | 6.73 | 4.7 | 5.06 | 6 | 7.4 | 5.3 | 7.12 | 6.88 | 5.46 | 4.77 | 8.73 | 7.39 | 10.11 | 9.9 | 10.45 | 11.91 | 11.66 | 9.96 | 13 | 14.92 | 13.64 | 14.55 |
| 5 | 57 | 4.01 | 4.94 | 4.28 | 4.35 | 4.73 | 6.33 | 3.85 | 6.2 | 5.43 | 4.01 | 5.48 | 3.53 | 5.58 | 4.09 | 4.27 | 3.88 | 12.78 | 4.55 | 3.46 | 3.83 | 3.9 | 3.44 |
| 5 | 58 | 7.77 | 13.35 | 13.18 | 8.13 | 14.28 | 8.22 | 9.19 | 14.37 | 16.21 | 10.76 | 17.46 | 9.64 | 18.89 | 12.1 | 20.31 | 12.14 | 10.48 | 17.07 | 20.83 | 13.15 | 21.61 | 13.98 |
| 5 | 59 | 4.12 | 4.48 | 4.25 | 5 | 4.03 | 5.05 | 4.37 | 6.06 | 8.19 | 7.22 | 6.54 | 6.13 | 9.32 | 9.7 | 6.14 | 6.92 | 6.6 | 7.21 | 9.63 | 13.39 | 14.81 | 11.07 |
| 5 | 60 | 9.7 | 8.58 | 8.6 | 9.13 | 9.31 | 8.17 | 7.44 | 8.49 | 9.64 | 7.9 | 9.31 | 7.92 | 10.26 | 9.23 | 9.75 | 9.23 | 7.94 | 8.65 | 8.33 | 9.96 | 10.47 | 9.66 |
| 6 | 61 | 6.82 | 6.46 | 6.59 | 6.94 | 7.69 | 7.42 | 7.42 | 8.01 | 8.7 | 7.96 | 9.44 | 8.46 | 10.68 | 10.4 | 12.49 | 10.79 | 10.16 | 9.84 | 11.05 | 12.89 | 14.96 | 12.71 |
| 6 | 62 | 4.92 | 4.6 | 4.39 | 4.7 | 5.76 | 4.43 | 6.19 | 4.31 | 5.9 | 4.93 | 6.2 | 5.62 | 6.39 | 6.19 | 8.53 | 6.89 | 6.32 | 7.34 | 2.95 | 8.1 | 9.89 | 8.11 |
| 6 | 63 | 9.81 | 8.57 | 10.51 | 8.01 | 10.51 | 8.99 | 9.38 | 10.24 | 12.08 | 10.31 | 12.44 | 9.47 | 13.8 | 10.89 | 14.01 | 11.48 | 10.3 | 13.32 | 11.04 | 15.53 | 16.66 | 12.54 |
| 6 | 64 | 5.49 | 4.6 | 6 | 4.06 | 6.26 | 4.62 | 3.92 | 5.99 | 8.26 | 6.58 | 7.8 | 5.92 | 9.84 | 7.34 | 10.1 | 7.97 | 7.97 | 9.16 | 10.69 | 12.28 | 14.75 | 13.16 |
| 6 | 65 | 9.3 | 7.24 | 9.91 | 7.86 | 9.99 | 8.34 | 10.59 | 8.13 | 12.15 | 10.83 | 12.62 | 10.59 | 14.37 | 12.27 | 14.96 | 12.79 | 11.07 | 14.3 | 12.98 | 16.64 | 18.7 | 15.76 |
| 6 | 66 | 6.33 | 6.13 | 7.04 | 7.37 | 6.49 | 7 | 6.68 | 8.23 | 10.54 | 8.11 | 9.17 | 7.88 | 13.19 | 10.01 | 13.53 | 10.67 | 9.79 | 13.22 | 11.63 | 14.52 | 15.87 | 12.99 |
| 6 | 67 | 11.04 | 9.33 | 9.11 | 10.8 | 11.9 | 8.92 | 11.6 | 8.52 | 13.57 | 11.01 | 14.56 | 12.47 | 15.41 | 13.2 | 16.09 | 12.5 | 10.88 | 15.16 | 12.63 | 17.44 | 14.04 | 18.36 |
| 6 | 68 | 5.96 | 3.96 | 5.07 | 4.74 | 4.3 | 5.71 | 5.52 | 7.34 | 8.3 | 6.39 | 8.98 | 6.33 | 10.91 | 8.6 | 10.9 | 8.98 | 7.62 | 8.25 | 9.7 | 11.43 | 13.98 | 12.23 |
| 6 | 69 | 3.72 | 6.91 | 6.15 | 3.83 | 3.66 | 3.93 | 5.97 | 2.9 | 6.87 | 4.64 | 4.11 | 2.86 | 6.08 | 4.75 | 7.01 | 5 | 6.68 | 5.11 | 9.24 | 6.8 | 7.66 | 10.69 |
| 6 | 70 | 5.34 | 4.53 | 4.66 | 5.11 | 5.87 | 5.65 | 7.47 | 6.97 | 7.82 | 6.61 | 7.56 | 6.02 | 8.17 | 7.51 | 9.9 | 8.34 | 7.42 | 9.11 | 9.18 | 12.12 | 13.39 | 10.73 |
| 6 | 71 | 8.94 | 9.19 | 8.99 | 9.19 | 9.94 | 10.06 | 8.75 | 9.25 | 10.98 | 10.19 | 11.64 | 10.33 | 12.6 | 11.64 | 12.05 | 11.82 | 11.03 | 10.47 | 11.69 | 12.15 | 12.46 | 12.27 |
| 6 | 72 | 5.44 | 4.16 | 5.49 | 4.02 | 6.27 | 5.2 | 4.46 | 5.1 | 7.16 | 6.11 | 6.62 | 5.59 | 7.77 | 6.9 | 9.15 | 8.25 | 8.52 | 8.03 | 10.05 | 11.17 | 13.51 | 11.9 |
| 7 | 73 | 5.14 | 5.89 | 7.07 | 4.42 | 6.69 | 6.4 | 5.57 | 5.56 | 7.27 | 7.49 | 8.24 | 5.5 | 7.97 | 6.91 | 9.22 | 6.81 | 5.31 | 6.14 | 7.4 | 6.85 | 9.34 | 6.27 |
| 7 | 74 | 3.73 | 3.54 | 4.89 | 4 | 4.68 | 5.11 | 5.87 | 6.03 | 7.02 | 6.52 | 6.48 | 6.47 | 7.38 | 6.99 | 9.07 | 9.21 | 8.06 | 7.01 | 9.67 | 8.5 | 10.02 | 10.26 |
| 7 | 75 | 6.56 | 7.7 | 8.19 | 8.93 | 8.99 | 8.33 | 9.93 | 12.05 | 11.57 | 10.46 | 12.93 | 10.96 | 13.55 | 12.91 | 16.86 | 13.64 | 11.23 | 15.82 | 22.18 | 17.03 | 21.06 | 15.7 |
| 7 | 76 | 8.1 | 7.18 | 7.65 | 8.7 | 10.41 | 9.42 | 9.19 | 8.9 | 11.68 | 10.55 | 15.83 | 12.19 | 19.36 | 13.82 | 12.49 | 10.27 | 9.88 | 11.45 | 22.24 | 15.6 | 25.55 | 15.85 |
| 7 | 77 | 12.47 | 7.06 | 13.84 | 8.69 | 13.66 | 8.61 | 8.85 | 13.11 | 15.37 | 9.72 | 15.76 | 10.32 | 17.14 | 11.42 | 17.77 | 12.71 | 11.55 | 16.02 | 12.12 | 17.06 | 18.09 | 13.08 |
| 7 | 78 | 7.13 | 6.2 | 6.19 | 9.41 | 9.68 | 9.03 | 10.73 | 9.22 | 14.01 | 12.06 | 14.29 | 9.36 | 4.59 | 12.76 | 18.67 | 13.85 | 17.26 | 14.56 | 16.31 | 21.44 | 21.77 | 17.17 |
| 7 | 79 | 4.88 | 5.23 | 5.63 | 6.06 | 6.64 | 6.52 | 6.35 | 6.23 | 7.74 | 8.21 | 9.1 | 7.52 | 10.24 | 8.77 | 14.62 | 13.84 | 12.17 | 12.56 | 15.01 | 14.76 | 14.7 | 14.45 |
| 7 | 80 | 5.98 | 7.5 | 7.6 | 8.5 | 7.65 | 7.08 | 7.24 | 7.58 | 9.27 | 8.19 | 9.06 | 7.65 | 9.96 | 10.14 | 12.08 | 10.21 | 9 | 10.96 | 10.89 | 13.23 | 14.65 | 12.09 |
| 7 | 81 | 8.55 | 7.61 | 8.32 | 9.61 | 8.65 | 9.78 | 10.33 | 8.6 | 12.19 | 10.46 | 11.63 | 10.14 | 13.17 | 11.66 | 14.48 | 13.89 | 12.07 | 15.04 | 13.43 | 16.15 | 17.68 | 13.8 |
| 7 | 82 | 13.57 | 13.13 | 14.97 | 14.63 | 15.81 | 14.44 | 14.57 | 16.03 | 15.44 | 16.73 | 17.19 | 15.63 | 19.51 | 18.84 | 19.88 | 19.05 | 14.79 | 19.22 | 21.24 | 17.45 | 20.1 | 23.22 |
| 7 | 83 | 7.51 | 8.53 | 8.47 | 8.66 | 9.64 | 8.21 | 8.73 | 8.83 | 10.54 | 9.19 | 12.05 | 9.04 | 12.2 | 10.6 | 13.11 | 11.42 | 9.75 | 12.22 | 11.63 | 13.48 | 14.51 | 11.88 |
| 7 | 84 | 5.3 | 4.04 | 4.65 | 5.18 | 5.9 | 6.05 | 7.19 | 6.44 | 8.44 | 7.35 | 7.2 | 6.37 | 10.57 | 8.4 | 10.62 | 11.11 | 6.7 | 9.94 | 8.65 | 11.02 | 11.18 | 8.55 |
| 8 | 85 | 4.13 | 5.25 | 3.91 | 3.13 | 5.67 | 6.5 | 5.54 | 3.63 | 7.09 | 6.6 | 5.82 | 4.57 | 6.85 | 5.85 | 6.26 | 6.31 | 6.31 | 5.82 | 7.63 | 6.63 | 8.49 | 7.6 |
| 8 | 86 | 5.5 | 4.8 | 5.19 | 4.33 | 10.31 | 5.89 | 6.34 | 4.43 | 12.06 | 8.69 | 6.59 | 4.62 | 7.77 | 6.98 | 7.81 | 6.97 | 7.17 | 6.54 | 8.52 | 9.74 | 11.8 | 9.9 |
| 8 | 87 | 3.58 | 4.11 | 3.73 | 4.44 | 6.08 | 4.28 | 4.69 | 5.55 | 6.47 | 5.2 | 9.94 | 7.62 | 8.04 | 6.13 | 7.69 | 6.22 | 6.79 | 7.66 | 8.35 | 9.68 | 11 | 9.4 |
| 8 | 88 | 7.66 | 4.17 | 7.99 | 4.76 | 5.31 | 5.31 | 5.09 | 8.64 | 8.99 | 6.4 | 10.47 | 5.88 | 10.25 | 5.99 | 10.58 | 7.14 | 6.66 | 9.97 | 8.02 | 11.95 | 13.61 | 8.06 |
| 8 | 89 | 4.84 | 6.9 | 5.26 | 7.88 | 9.43 | 5.81 | 6.11 | 9.03 | 7.31 | 6.19 | 12.93 | 8.18 | 13.08 | 10.51 | 13.92 | 10.86 | 12.97 | 10.52 | 12.94 | 16.14 | 17.29 | 14.33 |
| 8 | 90 | 5.04 | 6.53 | 5.82 | 6.16 | 6.11 | 5.35 | 4.64 | 6.47 | 7.9 | 6.96 | 8.72 | 5.87 | 8.22 | 7.34 | 8.58 | 6.64 | 8.04 | 6.3 | 8.54 | 10.85 | 12.08 | 9.59 |
| 8 | 91 | 6.75 | 5.46 | 7.4 | 5.38 | 7.02 | 7.02 | 8.44 | 8.42 | 9.04 | 6.66 | 9.28 | 9.24 | 11.05 | 7.99 | 9.86 | 10.8 | 9.5 | 9.56 | 11.17 | 10.96 | 12.65 | 13.3 |
| 8 | 92 | 6.56 | 4.56 | 6.26 | 6.7 | 7.8 | 6.67 | 7.28 | 6.73 | 9.31 | 7.38 | 9.92 | 7.18 | 10.61 | 8.24 | 11.54 | 9.32 | 10.43 | 10.19 | 11.76 | 10.26 | 13.44 | 10.55 |
| 8 | 93 | 10.32 | 11.25 | 9.48 | 11.54 | 12.53 | 9.89 | 11.31 | 9.47 | 12.52 | 10.02 | 7.59 | 5.11 | 13.76 | 9.84 | 14.22 | 12.03 | 13.13 | 12.4 | 11 | 13.99 | 14.89 | 12.07 |
| 8 | 94 | 6.92 | 4.94 | 6.55 | 6.51 | 6.14 | 7.16 | 5.96 | 7.26 | 9.12 | 8.71 | 5.82 | 4.57 | 10.52 | 8.03 | 9.7 | 7.6 | 8.43 | 9.29 | 8.2 | 9.18 | 10.47 | 13.37 |
| 8 | 95 | 15.76 | 9.75 | 8.81 | 15.01 | 15.14 | 9.2 | 15.76 | 7.7 | | | | | | | | | | | | | | |
| 8 | 96 | 6.51 | 6.75 | 6.9 | 6.96 | 7.43 | 10.26 | 11.1 | 8.77 | 11.54 | 9.42 | 11.75 | 9.1 | 13.44 | 10.24 | 13.32 | 10.93 | 10.06 | 11.42 | 13.82 | 12.28 | 13.16 | 15.81 |

APPENDIX 2.3
Tumor Weights (in grams)

| Group | Animal # | Weight |
|---|---|---|
| 1 | 1 | 4.83 |
| 1 | 2 | 2.2 |
| 1 | 3 | 3.084 |
| 1 | 4 | 4.042 |
| 1 | 5 | 1.38 |
| 1 | 6 | 5.76 |
| 1 | 7 | 4.778 |
| 1 | 8 | 3.488 |
| 1 | 9 | 1.78 |
| 1 | 10 | 3.667 |
| 1 | 11 | 0.416 |
| 1 | 12 | 10.6 |
| 2 | 13 | 1.85 |
| 2 | 14 | |
| 2 | 15 | 2.95 |
| 2 | 16 | 5.84 |
| 2 | 17 | 2.56 |
| 2 | 18 | 1.56 |
| 2 | 19 | 7.102 |
| 2 | 20 | 16.56 |
| 2 | 21 | 3.878 |
| 2 | 22 | 4.74 |
| 2 | 23 | 0.866 |
| 2 | 24 | 2.821 |
| 3 | 25 | 3.26 |
| 3 | 26 | 2.64 |
| 3 | 27 | 3.126 |
| 3 | 28 | 1.674 |
| 3 | 29 | 3.22 |
| 3 | 30 | 5.65 |
| 3 | 31 | 5.194 |
| 3 | 32 | 5.36 |
| 3 | 33 | 5.35 |
| 3 | 34 | 0.994 |
| 3 | 35 | 1.01 |
| 3 | 36 | 2.91 |
| 4 | 37 | 3.7 |
| 4 | 38 | 5.116 |
| 4 | 39 | 0.928 |
| 4 | 40 | 3.142 |
| 4 | 41 | 4.344 |
| 4 | 42 | 9.198 |
| 4 | 43 | 0.766 |
| 4 | 44 | 1.77 |
| 4 | 45 | 0.332 |
| 4 | 46 | 1.31 |
| 4 | 47 | 1.448 |
| 4 | 48 | 1.38 |
| 5 | 49 | 0.74 |
| 5 | 50 | 1.36 |
| 5 | 51 | 0.47 |
| 5 | 52 | 2.43 |
| 5 | 53 | 1.04 |
| 5 | 54 | 0.41 |
| 5 | 55 | 0.66 |
| 5 | 56 | 0.7 |
| 5 | 57 | 0.02 |
| 5 | 58 | 1.19 |
| 5 | 59 | 0.57 |
| 5 | 60 | 0.35 |
| 6 | 61 | 0.76 |
| 6 | 62 | 0.17 |
| 6 | 63 | 0.98 |
| 6 | 64 | 0.83 |
| 6 | 65 | 1.76 |
| 6 | 66 | 0.83 |
| 6 | 67 | 1.04 |
| 6 | 68 | 0.63 |
| 6 | 69 | 0.23 |
| 6 | 70 | 0.51 |
| 6 | 71 | 0.68 |
| 6 | 72 | 0.45 |
| 7 | 73 | 0.1 |
| 7 | 74 | 0.3 |
| 7 | 75 | 2.83 |
| 7 | 76 | 3.93 |
| 7 | 77 | 1.32 |
| 7 | 78 | 3.3 |
| 7 | 79 | 1.22 |
| 7 | 80 | 0.72 |
| 7 | 81 | 1.24 |
| 7 | 82 | 3.52 |
| 7 | 83 | 0.91 |
| 7 | 84 | 0.43 |
| 8 | 85 | 0.31 |
| 8 | 86 | 0.39 |
| 8 | 87 | 0.31 |
| 8 | 88 | 0.33 |
| 8 | 89 | 1.05 |
| 8 | 90 | 0.49 |
| 8 | 91 | 0.64 |
| 8 | 92 | 0.48 |
| 8 | 93 | 0.67 |
| 8 | 94 | 0.62 |
| 8 | 95 | |
| 8 | 96 | 0.89 |

### Example 3

### An Anti-Tumor Efficacy Study of Combined Treatment of SCV-07 and Cisplatin in Mice Bearing Subcutaneous LLC Tumor

### Abbreviations

BW Body Weight

CO₂ Carbon Dioxide

CDDP Cis-Diamine Dichloroplatinum (Cisplatin)

g Gram

kg Kilogram

L Length

LLC Lewis Lung Carcinoma

mg Milligram

mL Milliliter

PBS Phosphate Buffered Saline

PI Percent Inhibition

SD Standard Deviation

TV Tumor Volume

TW Tumor Weight

VBI Vital Bridge (China), Inc.

W Width

### Summary

In this study, the inhibitory effect of SCV-07 along with cisplatin (CDDP) was evaluated on the growth of a subcutaneous cancer grown from murine Lewis lung carcinoma (LLC) cells inoculated in C57/BL6 mice. A total of 70 mice were implanted subcutaneously with murine LLC cells, followed by treatment with SCV-07 or CDDP alone or in combination for 14 consecutive days. SCV-07 was administered daily by sc injection, while CDDP was administered by ip injection on days 1, 6, and 12. In total, 7 groups were used: Group 1: vehicle; Group 2: CDDP 2 mg/kg; Group 3: CDDP 6 mg/kg; Group 4: SCV-07 10 mg/kg; Group 5: SCV-07 20 mg/kg; Group 6: SCV-07 10 mg/kg plus CDDP 2 mg/kg; Group 7: SCV-07 20 mg/kg plus CDDP 2 mg/kg. Body weights were recorded once every 3 days, tumor sizes were measured once every other days, and tumor weights were measured on Day 16 (necropsy day) at the end of the study.

Throughout the course of the study, no animal died. The statistical results of body weights showed no significant differences between either of SCV-07 treatment alone groups (Groups 4 and 5) and the vehicle control group (Group 1), indicating no effect of SCV-07 on animal growth. By contrast, from Day 3 onwards, the group receiving 6 mg/kg CDDP treatment (Group 3) consistently showed statistically significant decreases in body weight. Group 2, the group receiving 2 mg/kg CDDP treatment, showed statistically significant decreases in body weight only on Day 15, while Group 7, which received 2 mg/kg CDDP in combination with 20 mg/kg SCV-07, consistently exhibited statistically significant decreases in body weight relative to Group 1 from Day 3 onwards.

Tumor measurement data showed that the mean tumor volumes of Group 2 and Group 3 were statistically significantly smaller than that of Group 1 on Day 6. On Days 8, 10, 12 and 14, the mean tumor volumes of all groups were statistically significantly smaller than Group 1. On Day 16, the mean tumor weights of all treatment groups were lower than Group 1. The tumor inhibition calculated based on tumor weight were 58.90% (p<0.01), 77.35% (p<0.01), 16.84% (p<0.05), 37.45% (p<0.01), 40.81% (p<0.01) and 56.13% (p<0.01), for Group 2, Group 3, Group 4, Group 5, Group 6, and Group 7, respectively.

In summary, the tumor model used in this study was valid as the positive control drug CDDP effectively reduced the tumor growth. Treatment with SCV-07 (10 mg/kg or 20 mg/kg) inhibited tumor growth as reflected by the smaller tumor volumes and lower tumor weights in these groups relative to those of the vehicle control group. The treatment regimens of SCV-07 (10 or 20 mg/kg) in combination with CDDP (2 mg/kg) led to higher inhibition of tumor growth than SCV-07 treatment alone, but without increased anti-tumor efficacy compared to CDDP alone (no additive effect).

### Introduction

This study was designed to evaluate the anti-tumor effect of SCV-07 used alone or in combination with CDDP in murine Lewis lung cancer (LLC) model in order to explore its therapeutic potential for the treatment of lung cancer. CDDP was also used as the positive control drug to validate the cancer model.

### Materials and Methods

### Test and Control Articles

PBS was used as the negative control article (vehicle), and CDDP as the positive control. CDDP was purchased from PUMC hospital. Manufactured by Qilu Pharmaceutical Co., LTD, each vial of the medicine contains 10 mg CDDP powder. Prior to use, PBS was added to one vial of CDDP to achieve the proper dose level as indicated in the dose formulation table (Table 3.1). The formulation was kept on ice, protected from light, and used immediately. Test article (SCV-07) was dissolved in PBS to achieve the proper dose levels as indicated on table 1; kept on ice, protected from light, and used within one week.

TABLE 3.1
Dose Formulation

| | | Dose | |
|---|---|---|---|
| | Dose level | Volume | Concentration |
| Treatment | (mg/kg) | (mL/kg) | (mg/mL) |
| SCV-07 | 10 | 5 | 2 |
| | 20 | 5 | 4 |
| CDDP | 2 | 5 | 0.4 |
| | 6 | 5 | 1.2 |

### Test System and Animal Husbandry

### Murine Lung Cancer Cells (LLC)

Murine Lewis lung cancer cells were obtained from the Cell Culture Center of Chinese Academy of Medical Sciences (CAMS; Beijing, P. R. China). The cancer cells were adapted in C57BL/6 mice before used in experiment. Refer to Section 4.3.1 for details on cell adaptation.

### Test System

Thirty-five male and thirty-five female, healthy, naive, C57BL/6 mice were received from the Institute of Laboratory Animal Science, CAMS, Beijing, P. R. China. The animals were six weeks old and weighed between 18 and 22 grams at the start of the study.

### Animal Husbandry

Animals were group-housed in autoclaved shoe box cages with autoclaved wood chips as the bedding materials. The temperature of the animal room was maintained at 22 to 25° C., and the relative humidity was maintained at 40 to 60%. A 12-hour light/12-hour dark cycle was maintained except when interrupted by study-related events. Animals were fed ad libitum with sterile water and Beijing KeAoXieLi Rodent Diet (certified). All animals were acclimated for 3 days before tumor inoculation.

### Experimental Procedures

### Tumor Cell Adaptation

One vial of LLC cells was removed from the liquid nitrogen stock, and placed into a 37° C. water bath. Gentle swirling was conducted until the content of the vial was thawed. Using aseptic tissue culture technique, the cells were immediately centrifuged with a TD5A-WS centrifuge at 1000 rpm, 20-25° C., for 5 min. After centrifugation, the cells were suspended in 0.1 to 0.5 mL normal saline (NS) and then subcutaneously injected into 10 mice (0.1 mL/mouse, about 1×10⁶ cells). After 1 or 2 weeks, when the tumor diameter was approximately 1 cm, the animals were euthanized with CO₂ overdose and the tumors excised. The procedure was repeated with another 20 mice to generate a sufficient number of LLC cells with adequate transplantability.

### Tumor Cell Inoculation

On the day of tumor implantation, approximately 1.2×10⁶ cells in 0.1 mL were subcutaneously injected on the right axillary area of each mouse. The day of tumor implantation was defined as Day 0.

### Study Design and Treatment Regimen

On Day 1, the animals were randomly assigned into different weight-matched groups, and dosing was started using the regimen according to Table 3.2. Briefly, SCV-07 was administered once daily via subcutaneous injection for 14 consecutive days at a site different from that of tumor cell implantation, while CDDP was intraperitoneally administered on Days 1, 6, and 12.

### Evaluation of Anti-Tumor Effect

From Day 1 to Day 14, mortality and morbidity were checked twice daily, the body weights were recorded once every 3 days, and tumors were measured using a caliper once every 2 days. At the end of the study (Day 16), the animals were euthanized by CO₂ asphyxiation, and the tumors were excised and weighed.

Tumor volume was calculated using the following formula:

Tumor Volume=Length×Width×Width/2

Tumor volume inhibition (PI) was calculated according to the formula below:

PI(TV)=(TV_{vehicle}−TV_{drug treated})/TV_{vehicle}×100%

Where, TV is the tumor volume on the day of measurement, “vehicle” denotes the group receiving PBS, and “drug treated” denotes groups receiving SCV-07 and/or CDDP.

The anti-tumor effect of SCV-07 used alone or in combination with CDDP was also evaluated by tumor weight. The tumor weight of each mouse was recorded after euthanasia, and the percent inhibition of tumor weight was calculated according to the formula below:

PI(TW)=(TW_{vehicle}−TW_{drug treated})/TW_{vehicle}×100%

Mean and standard deviations were calculated using Excel.

TABLE 3.2
Treatment Regimen and Study Design

| Group | | | Number of | Dosing | Necropsy |
|---|---|---|---|---|---|
| Number | Group Name | Treatment | Animals | Days | Day |
| 1 | Vehicle Control | PBS | 10 | Days 1-14 | Day 16 |
| 2 | Positive Control 1 | CDDP 2 mg/kg, i.p., on | 10 | | |
| | | days 1, 6, and 12 | | | |
| 3 | Positive Control 2 | CDDP 6 mg/kg, i.p., on | 10 | | |
| | | days 1, 6, and 12 | | | |
| 4 | Test Article 1 | SCV-07, 10 mg/kg, s.c., | 10 | | |
| | | daily | | | |
| 5 | Test Article 2 | SCV-07, 20 mg/kg, s.c., | 10 | | |
| | | daily | | | |
| 6 | Combination 1 | SCV-07, 10 mg/kg, s.c., | 10 | | |
| | | daily + CDDP 2 mg/kg, | | | |
| | | i.p., on days 1, 6, and 12 | | | |
| 7 | Combination 2 | SCV-07, 20 mg/kg, s.c., | 10 | | |
| | | daily + CDDP 2 mg/kg, | | | |
| | | i.p., on days 1, 6, and 12 | | | |

### Statistical Analysis

Inter-group comparisons were performed in terms of tumor volume, tumor weight and body weight, using a student's t test. P values of less than 0.05 were considered to be statistically significant.

### Results and Discussion

### Mortality

Throughout the course of the study, no animal died.

### Tumor Size

Raw measurement data of tumor size were tabulated in Appendixes 3.1-3.14. The calculated mean tumor volumes and statistical testing results of each treatment group versus the vehicle group were tabulated in the Tables 3-9. Tumors were not measurable in any group on day 2. On day 4, mean tumor volumes in all of groups except Group 4 were statistically significantly smaller than that of Group 1 (vehicle control). On Day 6, only Group 2 and Group 3 showed the smaller mean tumor volumes. On Days 8, 10, 12 and 14, the mean tumor volumes of all groups were statistically significantly smaller than Group 1.

### Tumor Weight

Raw data of tumor weights measured on Day 16 were tabulated in Appendix 3.15. The calculated percent inhibition (PI) values based on tumor weight and the statistical comparison results between each of treatment groups and the vehicle group were tabulated in Table 3.10. As shown in Table 3.10, the mean tumor weights of all treatment groups were lower than that of the vehicle group. The PI values of Group 2, Group 3, Group 4, Group 5, Group 6 and Group 7 were 58.90% (p<0.01), 77.35% (p<0.01), 16.84% (p<0.05), 37.45% (p<0.01), 40.81% (p<0.01) and 56.13% (p<0.01), respectively. Although the combination treatment groups (i.e., Groups 6 and 7) had higher PI values than the corresponding groups (i.e., Groups 4 and 5) that received SCV-07 treatment alone, these PI values were not higher than that of Group 2 which received CDDP treatment alone. There were no statistically significant differences in PI between either of the combination treatment groups and the CDDP treatment alone group (Group 2), indicating no additive effect when using CDDP in combination with SCV-07.

### Body Weight

Raw data of body weight measurement were listed in Appendixes 3.16-3.21. The results of statistical comparison of each treatment group versus the vehicle group were tabulated in the Tables 3.11-3.16.

As shown in the Tables 3.11-3.16, there were no statistically significant differences between each of the treatment groups and the vehicle control group on Day 0. On Day 3, Group 3 (CDDP 6 mg/kg) and Group 7 (CDDP 2 mg/kg+SCV-07 20 mg/kg) exhibited decreases in body weights by 9.33% (P<0.01) and 8.31% (P<0.01), respectively, relative to the vehicle group. There were no statistically significant differences in body weight for other groups in comparison to the vehicle group. On Day 6, the body weights of Groups 3 and 7 were 10.45% (p<0.01) and 6.58% (p<0.01) lower than the vehicle group. On Day 9, the body weights of Groups 3, 6, and 7 were 14.51% (p<0.01), 8.70% (p<0.05), and 11.41% (p<0.01) lower than the vehicle group, respectively. On Day 12, the body weights of Groups 3 and 7 were 13.62% (P<0.01) and 6.65% (P<0.05) lower than the vehicle group, respectively. On Day 15, the body weights of Groups 2, 3, 6, and 7 were 12.51% (p<0.01), 24.38% (P<0.01), 10.42% (P<0.05), and 14.56% (P<0.01) lower than the vehicle group, respectively. Throughout the course of the study, there were no statistically significant differences in body weights of Groups 4 and 5 that received SCV-07 treatment alone, indicating that SCV-07 had no significant effect on animal body weight, when used alone. On the contrary, the CDDP treatment was associated with the loss of body weight, and the observation was especially pronounced in Group 3, the group receiving high dose (6 mg/kg) of CDDP. The combined use of CDDP with SCV-07 did not alleviate the weight loss, but may in fact make the CDDP-associated weight loss more pronounced. Higher weight loss was noted in Group 7, the group receiving CDDP in combination with high dose of SCV-07, than in Group 2 which received CDDP alone.

### Conclusion and Discussion

In conclusion, the tumor model used in this study was valid as tumor growth was inhibited by positive control drug CDDP. Daily administration of test article SCV-07 at 10 mg/kg and 20 mg/kg was effective against the tumor growth. Mean tumor volumes in animals of all SCV-07-treated groups were significantly reduced in comparison to that of the vehicle control group from Day 8 onwards. Tumor weights, which were measured on Day 16, were also significantly reduced in the groups receiving 10 mg/kg or 20 mg/kg SCV-07 alone and in the groups receiving combination therapy. The combined use of 10 mg/kg or 20 mg/kg SCV-07 with 2 mg/kg CDDP produced 40.81% and 56.13% inhibition of tumor growth, in comparison to 16.84% and 37.45% inhibition obtained when using 10 mg/kg or 20 mg/kg SCV-07 alone, and 58.90% and 77.35% inhibition obtained when using 2 mg/kg or 6 mg/kg CDDP alone. These results suggested that the combined use of SCV-07 and CDDP produced no additive effect towards the inhibition of the tumor growth.

The mean body weights in CDDP treatment groups were significantly decreased, indicating a toxic effect. When used alone, SCV-07 did not cause any statistically significant loss of body weights in all SCV-07-treated groups, indicating that SCV-07 had no effect on animal body weight. However, when SCV-07 was used in combination with CDDP, it may exacerbate the CDDP-associated weight loss, as higher weight losses were noted in Group 7, the group receiving CDDP in combination with higher dose of SCV-07 throughout the course of the study.

### Tables

TABLE 3.3
Mean tumor volume (cm³) on Day 2

| | | | | Tumor | | |
|---|---|---|---|---|---|---|
| | | | Number of | Volume | | |
| Group | Group | | Survived | (Mean ± | PI | P |
| Number | Name | Treatment | Animals | SD) | (TV) | Value |
| 1 | Vehicle | PBS | 10 | 0 | — | — |
| | Control | | | | | |
| 2 | Positive | CDDP | 10 | 0 | — | — |
| | Control 1 | 2 mg/kg | | | | |
| 3 | Positive | CDDP | 10 | 0 | — | — |
| | Control 2 | 6 mg/kg | | | | |
| 4 | Test Article 1 | SCV-07 | 10 | 0 | — | — |
| | | 10 mg/kg | | | | |
| 5 | Test Article 2 | SCV-07 | 10 | 0 | — | — |
| | | 20 mg/kg | | | | |
| 6 | Combination 1 | SCV-07, 10 mg/kg + | 10 | 0 | — | — |
| | | CDDP 2 mg/kg | | | | |
| 7 | Combination 2 | SCV-07, 20 mg/kg + | 10 | 0 | — | — |
| | | CDDP 2 mg/kg | | | | |

TABLE 3.4
Mean tumor volume (cm³) on Day 4

| | | | Number of | Tumor | | |
|---|---|---|---|---|---|---|
| Group | Group | | Survived | Volume | PI | P |
| Number | Name | Treatment | Animals | (Mean ± SD) | (TV) | Value |
| 1 | Vehicle | PBS | 10 | 0.007 ± 0.0024 | | |
| | Control | | | | | |
| 2 | Positive | CDDP | 10 | 0.000 ± 0.0006 | 93.90 | 0.0000 |
| | Control 1 | 2 mg/kg | | | | |
| 3 | Positive | CDDP | 10 | 0.000 ± 0.0002 | 98.66 | 0.0000 |
| | Control 2 | 6 mg/kg | | | | |
| 4 | Test Article 1 | SCV-07 | 10 | 0.006 ± 0.0040 | 16.97 | 0.3989 |
| | | 10 mg/kg | | | | |
| 5 | Test Article 2 | SCV-07 | 10 | 0.004 ± 0.0016 | 42.01 | 0.0032 |
| | | 20 mg/kg | | | | |
| 6 | Combination 1 | SCV-07, 10 mg/kg + | 10 | 0.004 ± 0.0039 | 51.10 | 0.0168 |
| | | CDDP 2 mg/kg | | | | |
| 7 | Combination 2 | SCV-07, 20 mg/kg + | 10 | 0.003 ± 0.0013 | 54.15 | 0.0002 |
| | | CDDP 2 mg/kg | | | | |

TABLE 3.5
Mean tumor volume (cm³) on Day 6

| | | | Number of | Tumor | | |
|---|---|---|---|---|---|---|
| Group | Group | | Survived | Volume | PI | P |
| Number | Name | Treatment | Animals | (Mean ± SD) | (TV) | Value |
| 1 | Vehicle | PBS | 10 | 0.071 ± 0.0222 | | |
| | Control | | | | | |
| 2 | Positive | CDDP | 10 | 0.015 ± 0.0072 | 78.57 | 0.0000 |
| | Control 1 | 2 mg/kg | | | | |
| 3 | Positive | CDDP | 10 | 0.004 ± 0.0039 | 93.76 | 0.0000 |
| | Control 2 | 6 mg/kg | | | | |
| 4 | Test Article 1 | SCV-07 | 10 | 0.096 ± 0.0348 | −35.62 | 0.0689 |
| | | 10 mg/kg | | | | |
| 5 | Test Article 2 | SCV-07 | 10 | 0.069 ± 0.0229 | 3.31 | 0.8185 |
| | | 20 mg/kg | | | | |
| 6 | Combination 1 | SCV-07, 10 mg/kg + | 10 | 0.069 ± 0.0229 | 3.31 | 0.8185 |
| | | CDDP 2 mg/kg | | | | |
| 7 | Combination 2 | SCV-07, 20 mg/kg | 10 | 0.055 ± 0.0241 | 22.63 | 0.1384 |
| | | CDDP 2 mg/kg | | | | |

TABLE 3.6
Mean tumor volume (cm³) on Day 8

| | | | Number of | Tumor | | |
|---|---|---|---|---|---|---|
| Group | Group | | Survived | Volume | PI | P |
| Number | Name | Treatment | Animals | (Mean ± SD) | (TV) | Value |
| 1 | Vehicle | PBS | 10 | 0.449 ± 0.0880 | | |
| | Control | | | | | |
| 2 | Positive | CDDP | 10 | 0.089 ± 0.0458 | 80.19 | 0.0000 |
| | Control 1 | 2 mg/kg | | | | |
| 3 | Positive | CDDP | 10 | 0.035 ± 0.0291 | 92.13 | 0.0000 |
| | Control 2 | 6 mg/kg | | | | |
| 4 | Test Article 1 | SCV-07 | 10 | 0.329 ± 0.1108 | 26.73 | 0.0153 |
| | | 10 mg/kg | | | | |
| 5 | Test Article 2 | SCV-07 | 10 | 0.270 ± 0.0929 | 39.73 | 0.0003 |
| | | 20 mg/kg | | | | |
| 6 | Combination 1 | SCV-07, 10 mg/kg + | 10 | 0.185 ± 0.0504 | 58.70 | 0.0000 |
| | | CDDP 2 mg/kg | | | | |
| 7 | Combination 2 | SCV-07, 20 mg/kg + | 10 | 0.161 ± 0.0449 | 64.12 | 0.0000 |
| | | CDDP 2 mg/kg | | | | |

TABLE 3.7
Mean tumor volume (cm³) on Day 10

| | | | Number of | Tumor | | |
|---|---|---|---|---|---|---|
| Group | Group | | Survived | Volume | PI | P |
| Number | Name | Treatment | Animals | (Mean ± SD) | (TV) | Value |
| 1 | Vehicle | PBS | 10 | 1.102 ± 0.2442 | | |
| | Control | | | | | |
| 2 | Positive | CDDP | 10 | 0.221 ± 0.0873 | 79.98 | 0.0000 |
| | Control 1 | 2 mg/kg | | | | |
| 3 | Positive | CDDP | 10 | 0.092 ± 0.0658 | 91.67 | 0.0000 |
| | Control 2 | 6 mg/kg | | | | |
| 4 | Test Article 1 | SCV-07 | 10 | 0.695 ± 0.2090 | 36.92 | 0.0008 |
| | | 10 mg/kg | | | | |
| 5 | Test Article 2 | SCV-07 | 10 | 0.621 ± 0.1795 | 43.70 | 0.0001 |
| | | 20 mg/kg | | | | |
| 6 | Combination 1 | SCV-07, 10 mg/kg + | 10 | 0.402 ± 0.1256 | 63.55 | 0.0000 |
| | | CDDP 2 mg/kg | | | | |
| 7 | Combination 2 | SCV-07, 20 mg/kg + | 10 | 0.393 ± 0.0892 | 64.37 | 0.0000 |
| | | CDDP 2 mg/kg | | | | |

TABLE 3.8
Mean tumor volume (cm³) on Day 12

| | | | Number of | Tumor | | |
|---|---|---|---|---|---|---|
| Group | Group | | Survived | Volume | PI | P |
| Number | Name | Treatment | Animals | (Mean ± SD) | (TV) | Value |
| 1 | Vehicle | PBS | 10 | 1.565 ± 0.3264 | | |
| | Control | | | | | |
| 2 | Positive | CDDP | 10 | 0.481 ± 0.0951 | 69.28 | 0.0000 |
| | Control 1 | 2 mg/kg | | | | |
| 3 | Positive | CDDP | 10 | 0.185 ± 0.1165 | 88.16 | 0.0000 |
| | Control 2 | 6 mg/kg | | | | |
| 4 | Test Article 1 | SCV-07 | 10 | 1.081 ± 0.3261 | 30.92 | 0.0038 |
| | | 10 mg/kg | | | | |
| 5 | Test Article 2 | SCV-07 | 10 | 0.902 ± 0.3294 | 42.41 | 0.0003 |
| | | 20 mg/kg | | | | |
| 6 | Combination 1 | SCV-07, 10 mg/kg + | 10 | 0.649 ± 0.1757 | 58.55 | 0.0000 |
| | | CDDP 2 mg/kg | | | | |
| 7 | Combination 2 | SCV-07, 20 mg/kg + | 10 | 0.648 ± 0.2096 | 58.58 | 0.0000 |
| | | CDDP 2 mg/kg | | | | |

TABLE 3.9
Mean tumor volume (cm³) on Day 14

| | | | Number of | Tumor | | |
|---|---|---|---|---|---|---|
| Group | Group | | Survived | Volume | PI | P |
| Number | Name | Treatment | Animals | (Mean ± SD) | (TV) | Value |
| 1 | Vehicle | PBS | 10 | 2.388 ± 0.5225 | | |
| | Control | | | | | |
| 2 | Positive | CDDP | 10 | 0.802 ± 0.0858 | 66.44 | 0.0000 |
| | Control 1 | 2 mg/kg | | | | |
| 3 | Positive | CDDP | 10 | 0.374 ± 0.2229 | 84.35 | 0.0000 |
| | Control 2 | 6 mg/kg | | | | |
| 4 | Test Article 1 | SCV-07 | 10 | 1.467 ± 0.3658 | 38.58 | 0.0002 |
| | | 10 mg/kg | | | | |
| 5 | Test Article 2 | SCV-07 | 10 | 1.259 ± 0.2956 | 47.30 | 0.0000 |
| | | 20 mg/kg | | | | |
| 6 | Combination 1 | SCV-07, 10 mg/kg + | 10 | 1.027 ± 0.2889 | 57.01 | 0.0000 |
| | | CDDP 2 mg/kg | | | | |
| 7 | Combination 2 | SCV-07, 20 mg/kg + | 10 | 0.907 ± 0.2647 | 62.03 | 0.0000 |
| | | CDDP 2 mg/kg | | | | |

TABLE 3.10
Mean tumor weight (g) on Day 16

| | | | | Tumor | | |
|---|---|---|---|---|---|---|
| | | | Number of | Weight | | |
| Group | Group | | Survived | (Mean ± | PI | P |
| Number | Name | Treatment | Animals | SD) | (TW) | Value |
| 1 | Vehicle | PBS | 10 | 4.08 ± 0.6159 | | |
| | Control | | | | | |
| 2 | Positive | CDDP | 10 | 1.68 ± 0.4358 | 58.90 | 0.0000 |
| | Control 1 | 2 mg/kg | | | | |
| 3 | Positive | CDDP | 10 | 0.92 ± 0.4679 | 77.35 | 0.0000 |
| | Control 2 | 6 mg/kg | | | | |
| 4 | Test Article 1 | SCV-07 | 10 | 3.39 ± 0.5793 | 16.84 | 0.0193 |
| | | 10 mg/kg | | | | |
| 5 | Test Article 2 | SCV-07 | 10 | 2.55 ± 0.4791 | 37.45 | 0.0000 |
| | | 20 mg/kg | | | | |
| 6 | Combination 1 | SCV-07, 10 mg/kg + | 10 | 2.42 ± 0.4954 | 40.81 | 0.0000 |
| | | CDDP 2 mg/kg | | | | |
| 7 | Combination 2 | SCV-07, 20 mg/kg + | 10 | 1.79 ± 0.5690 | 56.13 | 0.0000 |
| | | CDDP 2 mg/kg | | | | |

TABLE 3.11
Mean body weight (g) on Day 0

| | | | Number of | Tumor | | |
|---|---|---|---|---|---|---|
| Group | Group | | Survived | Volume | PI | P |
| Number | Name | Treatment | Animals | (Mean ± SD) | (BW) | Value |
| 1 | Vehicle | PBS | 10 | 17.40 ± 0.8069 | | |
| | Control | | | | | |
| 2 | Positive | CDDP | 10 | 17.52 ± 0.9402 | −0.69 | 0.7629 |
| | Control 1 | 2 mg/kg | | | | |
| 3 | Positive | CDDP | 10 | 17.57 ± 0.8970 | −0.98 | 0.6612 |
| | Control 2 | 6 mg/kg | | | | |
| 4 | Test Article 1 | SCV-07 | 10 | 17.39 ± 0.7156 | 0.06 | 0.9769 |
| | | 10 mg/kg | | | | |
| 5 | Test Article 2 | SCV-07 | 10 | 17.15 ± 0.5442 | 1.44 | 0.4272 |
| | | 20 mg/kg | | | | |
| 6 | Combination 1 | SCV-07, 10 mg/kg + | 10 | 17.10 ± 0.9250 | 1.72 | 0.4496 |
| | | CDDP 2 mg/kg | | | | |
| 7 | Combination 2 | SCV-07, 20 mg/kg + | 10 | 16.97 ± 0.6019 | 2.47 | 0.1935 |
| | | CDDP 2 mg/kg | | | | |

TABLE 3.12
Mean body weight (g) on Day 3

| | | | Number of | Tumor | | |
|---|---|---|---|---|---|---|
| Group | Group | | Survived | Volume | PI | P |
| Number | Name | Treatment | Animals | (Mean ± SD) | (BW) | Value |
| 1 | Vehicle | PBS | 10 | 17.57 ± 0.8486 | | |
| | Control | | | | | |
| 2 | Positive | CDDP | 10 | 16.56 ± 1.4592 | 5.75 | 0.0747 |
| | Control 1 | 2 mg/kg | | | | |
| 3 | Positive | CDDP | 10 | 15.93 ± 1.5762 | 9.33 | 0.0096 |
| | Control 2 | 6 mg/kg | | | | |
| 4 | Test Article 1 | SCV-07 | 10 | 17.67 ± 0.7469 | −0.57 | 0.7829 |
| | | 10 mg/kg | | | | |
| 5 | Test Article 2 | SCV-07 | 10 | 17.52 ± 0.3676 | 0.28 | 0.8662 |
| | | 20 mg/kg | | | | |
| 6 | Combination 1 | SCV-07, 10 mg/kg + | 10 | 16.65 ± 1.3277 | 5.24 | 0.0814 |
| | | CDDP 2 mg/kg | | | | |
| 7 | Combination 2 | SCV-07, 20 mg/kg + | 10 | 16.11 ± 0.9061 | 8.31 | 0.0016 |
| | | CDDP 2 mg/kg | | | | |

TABLE 3.13
Mean body weight (g) on Day 6

| | | | Number of | Tumor | | |
|---|---|---|---|---|---|---|
| Group | Group | | Survived | Volume | PI | P |
| Number | Name | Treatment | Animals | (Mean ± SD) | (BW) | Value |
| 1 | Vehicle | PBS | 10 | 18.09 ± 0.7795 | | |
| | Control | | | | | |
| 2 | Positive | CDDP | 10 | 17.52 ± 1.2639 | 3.15 | 0.2405 |
| | Control 1 | 2 mg/kg | | | | |
| 3 | Positive | CDDP | 10 | 16.20 ± 1.1402 | 10.45 | 0.0004 |
| | Control 2 | 6 mg/kg | | | | |
| 4 | Test Article 1 | SCV-07 | 10 | 18.12 ± 0.6250 | −0.17 | 0.9254 |
| | | 10 mg/kg | | | | |
| 5 | Test Article 2 | SCV-07 | 10 | 17.88 ± 0.4492 | 1.16 | 0.4700 |
| | | 20 mg/kg | | | | |
| 6 | Combination 1 | SCV-07, 10 mg/kg + | 10 | 17.51 ± 1.2512 | 3.21 | 0.2294 |
| | | CDDP 2 mg/kg | | | | |
| 7 | Combination 2 | SCV-07, 20 mg/kg + | 10 | 16.90 ± 1.0088 | 6.58 | 0.0085 |
| | | CDDP 2 mg/kg | | | | |

TABLE 3.14
Mean body weight (g) on Day 9

| | | | Number of | Tumor | | |
|---|---|---|---|---|---|---|
| Group | Group | | Survived | Volume | PI | P |
| Number | Name | Treatment | Animals | (Mean ± SD) | (BW) | Value |
| 1 | Vehicle | PBS | 10 | 18.40 ± 0.8511 | | |
| | Control | | | | | |
| 2 | Positive | CDDP | 10 | 17.74 ± 1.0926 | 3.59 | 0.1492 |
| | Control 1 | 2 mg/kg | | | | |
| 3 | Positive | CDDP | 10 | 15.73 ± 0.7558 | 14.51 | 0.0000 |
| | Control 2 | 6 mg/kg | | | | |
| 4 | Test Article 1 | SCV-07 | 10 | 18.59 ± 0.6173 | −1.03 | 0.5748 |
| | | 10 mg/kg | | | | |
| 5 | Test Article 2 | SCV-07 | 10 | 18.24 ± 0.5522 | 0.87 | 0.6240 |
| | | 20 mg/kg | | | | |
| 6 | Combination 1 | SCV-07, 10 mg/kg + | 10 | 16.80 ± 1.5463 | 8.70 | 0.0103 |
| | | CDDP 2 mg/kg | | | | |
| 7 | Combination 2 | SCV-07, 20 mg/kg + | 10 | 16.30 ± 0.7394 | 11.41 | 0.0000 |
| | | CDDP 2 mg/kg | | | | |

TABLE 3.15
Mean body weight (g) on Day 12

| | | | Number of | Tumor | | |
|---|---|---|---|---|---|---|
| Group | Group | | Survived | Volume | PI | P |
| Number | Name | Treatment | Animals | (Mean ± SD) | (BW) | Value |
| 1 | Vehicle | PBS | 10 | 19.24 ± 1.4789 | | |
| | Control | | | | | |
| 2 | Positive | CDDP | 10 | 18.23 ± 1.8233 | 5.25 | 0.1905 |
| | Control 1 | 2 mg/kg | | | | |
| 3 | Positive | CDDP | 10 | 16.62 ± 1.6164 | 13.62 | 0.0014 |
| | Control 2 | 6 mg/kg | | | | |
| 4 | Test Article 1 | SCV-07 | 10 | 18.89 ± 1.0429 | 1.82 | 0.5484 |
| | | 10 mg/kg | | | | |
| 5 | Test Article 2 | SCV-07 | 10 | 19.12 ± 0.8522 | 0.62 | 0.8266 |
| | | 20 mg/kg | | | | |
| 6 | Combination 1 | SCV-07, 10 mg/kg + | 10 | 18.29 ± 1.9116 | 4.94 | 0.2298 |
| | | CDDP 2 mg/kg | | | | |
| 7 | Combination 2 | SCV-07, 20 mg/kg + | 10 | 17.96 ± 1.1862 | 6.65 | 0.0468 |
| | | CDDP 2 mg/kg | | | | |

TABLE 3.16
Mean body weight (g) on Day 15

| | | | Number of | Tumor | | |
|---|---|---|---|---|---|---|
| Group | Group | | Survived | Volume | PI | P |
| Number | Name | Treatment | Animals | (Mean ± SD) | (BW) | Value |
| 1 | Vehicle | PBS | 10 | 20.06 ± 1.2057 | | |
| | Control | | | | | |
| 2 | Positive | CDDP | 10 | 17.55 ± 1.6216 | 12.51 | 0.0010 |
| | Control 1 | 2 mg/kg | | | | |
| 3 | Positive | CDDP | 10 | 15.17 ± 1.9810 | 24.38 | 0.0000 |
| | Control 2 | 6 mg/kg | | | | |
| 4 | Test Article 1 | SCV-07 | 10 | 19.01 ± 1.1939 | 5.23 | 0.0661 |
| | | 10 mg/kg | | | | |
| 5 | Test Article 2 | SCV-07 | 10 | 19.31 ± 0.8724 | 3.74 | 0.1284 |
| | | 20 mg/kg | | | | |
| 6 | Combination 1 | SCV-07, 10 mg/kg + | 10 | 17.97 ± 1.9647 | 10.42 | 0.0102 |
| | | CDDP 2 mg/kg | | | | |
| 7 | Combination 2 | SCV-07, 20 mg/kg + | 10 | 17.14 ± 1.1394 | 14.56 | 0.0000 |
| | | CDDP 2 mg/kg | | | | |

APPENDIX 3.1
Tumor measurements (cm) on Day 2

| | | F1 | | F2 | | F3 | | F4 | | F5 | | M1 | | M2 | | M3 | | M4 | | M5 | |
|---|---|---|---|---|---|---|---|---|---|---|---|---|---|---|---|---|---|---|---|---|---|
| Group | Treatment | L | W | L | W | L | W | L | W | L | W | L | W | L | W | L | W | L | W | L | W |
| 1 | Vehicle | — | — | — | — | — | — | — | — | — | — | — | — | — | — | — | — | — | — | — | — |
| | Control | | | | | | | | | | | | | | | | | | | | |
| 2 | CDDP 2 mg/kg | — | — | — | — | — | — | — | — | — | — | — | — | — | — | — | — | — | — | — | — |
| 3 | CDDP 6 mg/kg | — | — | — | — | — | — | — | — | — | — | — | — | — | — | — | — | — | — | — | — |
| 4 | SCV-07 10 mg/kg | — | — | — | — | — | — | — | — | — | — | — | — | — | — | — | — | — | — | — | — |
| 5 | SCV-07 20 mg/kg | — | — | — | — | — | — | — | — | — | — | — | — | — | — | — | — | — | — | — | — |
| 6 | SCV-07 10 mg/kg + | — | — | — | — | — | — | — | — | — | — | — | — | — | — | — | — | — | — | — | — |
| | CDDP 2 mg/kg | | | | | | | | | | | | | | | | | | | | |
| 7 | SCV-07 20 mg/kg + | — | — | — | — | — | — | — | — | — | — | — | — | — | — | — | — | — | — | — | — |
| | CDDP 2 mg/kg | | | | | | | | | | | | | | | | | | | | |

— Not measurable

APPENDIX 3.2
Tumor volumes* (cm³) on Day 2

| Group | Treatment | F1 | F2 | F3 | F4 | F5 | M1 | M2 | M3 | M4 | M5 |
|---|---|---|---|---|---|---|---|---|---|---|---|
| 1 | Vehicle | — | — | — | — | — | — | — | — | — | — |
| | Control | | | | | | | | | | |
| 2 | CDDP 2 mg/kg | — | — | — | — | — | — | — | — | — | — |
| 3 | CDDP 6 mg/kg | — | — | — | — | — | — | — | — | — | — |
| 4 | SCV-07 | — | — | — | — | — | — | — | — | — | — |
| | 10 mg/kg | | | | | | | | | | |
| 5 | SCV-07 | — | — | — | — | — | — | — | — | — | — |
| | 20 mg/kg | | | | | | | | | | |
| 6 | SCV-07 | — | — | — | — | — | — | — | — | — | — |
| | 10 mg/kg + | | | | | | | | | | |
| | CDP 2 mg/kg | | | | | | | | | | |
| 7 | SCV-07 | — | — | — | — | — | — | — | — | — | — |
| | 20 mg/kg + | | | | | | | | | | |
| | CDDP 2 mg/kg | | | | | | | | | | |

*Tumor volumes were calculated using the formula “Tumor Volume = Length × Width × Width/2” based on the data listed in Appendix 3.1.

APPENDIX 3.3
Tumor measurements (cm) on Day 4

| | | F1 | | F2 | | F3 | | F4 | | F5 | | M1 | |
|---|---|---|---|---|---|---|---|---|---|---|---|---|---|
| Group | Treatment | L | W | L | W | L | W | L | W | L | W | L | W |
| 1 | Vehicle | 0.2 | 0.2 | 0.2 | 0.2 | 0.2 | 0.2 | 0.26 | 0.26 | 0.26 | 0.26 | 0.26 | 0.26 |
| | Control | | | | | | | | | | | | |
| 2 | CDDP 2 mg/kg | — | — | — | — | 0.1 | 0.1 | — | — | 0.1 | 0.1 | 0.1 | 0.1 |
| 3 | CDDP 6 mg/kg | 0 | 0 | 0 | 0 | 0 | 0 | 0.1 | 0.1 | 0 | 0 | 0 | 0 |
| 4 | SCV-07 10 mg/kg | 0.2 | 0.2 | 0.3 | 0.3 | 0.2 | 0.2 | 0.2 | 0.2 | 0.2 | 0.2 | 0.3 | 0.3 |
| 5 | SCV-07 20 mg/kg | 0.2 | 0.2 | 0.2 | 0.2 | 0.2 | 0.2 | 0.26 | 0.26 | 0.2 | 0.2 | 0.2 | 0.2 |
| 6 | SCV-07 10 mg/kg + | 0.11 | 0.11 | 0.2 | 0.2 | 0.11 | 0.11 | 0.2 | 0.2 | 0.2 | 0.2 | 0.2 | 0.2 |
| | CDDP 2 mg/kg | | | | | | | | | | | | |
| 7 | SCV-07 20 mg/kg + | 0.15 | 0.15 | 0.2 | 0.2 | 0.2 | 0.2 | 0.2 | 0.2 | 0.2 | 0.2 | 0.2 | 0.2 |
| | CDDP 2 mg/kg | | | | | | | | | | | | |

| | | M2 | | M3 | | M4 | | M5 | |
|---|---|---|---|---|---|---|---|---|---|
| Group | Treatment | L | W | L | W | L | W | L | W |
| 1 | Vehicle | 0.26 | 0.26 | 0.26 | 0.26 | 0.27 | 0.27 | 0.26 | 0.26 |
| | Control | | | | | | | | |
| 2 | CDDP 2 mg/kg | 0.1 | 0.1 | 0.1 | 0.1 | 0.16 | 0.16 | — | — |
| 3 | CDDP 6 mg/kg | 0 | 0 | 0 | 0 | 0 | 0 | 0.1 | 0.1 |
| 4 | SCV-07 10 mg/kg | 0.24 | 0.24 | 0.2 | 0.2 | 0.2 | 0.2 | 0.2 | 0.2 |
| 5 | SCV-07 20 mg/kg | 0.17 | 0.17 | 0.2 | 0.2 | 0.2 | 0.2 | 0.2 | 0.2 |
| 6 | SCV-07 10 mg/kg + | 0.21 | 0.21 | 0.3 | 0.3 | 0.1 | 0.1 | 0.1 | 0.1 |
| | CDDP 2 mg/kg | | | | | | | | |
| 7 | SCV-07 20 mg/kg + | 0.2 | 0.2 | 0.2 | 0.2 | 0.2 | 0.2 | 0.1 | 0.1 |
| | CDDP 2 mg/kg | | | | | | | | |

— Not measurable

APPENDIX 3.4
Tumor volumes* (cm³) on Day 4

| Group | Treatment | F1 | F2 | F3 | F4 | F5 | M1 | M2 | M3 | M4 | M5 |
|---|---|---|---|---|---|---|---|---|---|---|---|
| 1 | Vehicle Control | 0.004 | 0.004 | 0.004 | 0.008788 | 0.008788 | 0.008788 | 0.008788 | 0.008788 | 0.0098415 | 0.008788 |
| 2 | CDDP 2 mg/kg | 0 | 0 | 0.0005 | 0 | 0.0005 | 0.0005 | 0.0005 | 0.0005 | 0.002048 | 0 |
| 3 | CDDP 6 mg/kg | 0 | 0 | 0 | 0.0005 | 0 | 0 | 0 | 0 | 0 | 0.0005 |
| 4 | SCV-07 10 mg/kg | 0.004 | 0.0135 | 0.004 | 0.004 | 0.004 | 0.0135 | 0.006912 | 0.004 | 0.004 | 0.004 |
| 5 | SCV-07 20 mg/kg | 0.004 | 0.004 | 0.004 | 0.008788 | 0.004 | 0.004 | 0.0024565 | 0.004 | 0.004 | 0.004 |
| 6 | SCV-07 10 mg/kg + | 0.0006655 | 0.004 | 0.0006655 | 0.004 | 0.004 | 0.004 | 0.0046305 | 0.0135 | 0.0005 | 0.0005 |
| | CDDP 2 mg/kg | | | | | | | | | | |
| 7 | SCV-07 20 mg/ | 0.0016875 | 0.004 | 0.004 | 0.004 | 0.004 | 0.004 | 0.004 | 0.004 | 0.004 | 0.0005 |
| | Ckg + DDP 2 mg/kg | | | | | | | | | | |

*Tumor volumes were calculated using the formula “Tumor Volume = Length × Width × Width/2” based on the data listed in Appendix 3.3.

APPENDIX 3.5
Tumor measurements (cm) on Day 6

| | | F1 | | F2 | | F3 | | F4 | | F5 | | M1 | | M2 | | M3 | | M4 | | M5 | |
|---|---|---|---|---|---|---|---|---|---|---|---|---|---|---|---|---|---|---|---|---|---|
| Group | Treatment | L | W | L | W | L | W | L | W | L | W | L | W | L | W | L | W | L | W | L | W |
| 1 | Vehicle | 0.54 | 0.54 | 0.5 | 0.5 | 0.4 | 0.4 | 0.5 | 0.5 | 0.5 | 0.5 | 0.53 | 0.53 | 0.52 | 0.52 | 0.5 | 0.5 | 0.59 | 0.59 | 0.6 | 0.6 |
| | Control | | | | | | | | | | | | | | | | | | | | |
| 2 | CDDP 2 mg/kg | 0.3 | 0.3 | 0.4 | 0.4 | 0.3 | 0.3 | 0.2 | 0.2 | 0.3 | 0.3 | 0.3 | 0.3 | 0.35 | 0.35 | 0.3 | 0.3 | 0.3 | 0.3 | 0.3 | 0.3 |
| 3 | CDDP 6 mg/kg | 0.18 | 0.18 | 0 | 0 | 0.2 | 0.2 | 0.25 | 0.25 | 0 | 0 | 0.2 | 0.2 | 0.2 | 0.2 | 0.2 | 0.2 | 0.2 | 0.2 | 0.3 | 0.3 |
| 4 | SCV-07 10 mg/kg | 0.5 | 0.5 | 0.6 | 0.6 | 0.6 | 0.6 | 0.5 | 0.5 | 0.6 | 0.6 | 0.6 | 0.6 | 0.5 | 0.5 | 0.7 | 0.7 | 0.6 | 0.6 | 0.5 | 0.5 |
| 5 | SCV-07 20 mg/kg | 0.5 | 0.5 | 0.5 | 0.5 | 0.6 | 0.6 | 0.5 | 0.5 | 0.4 | 0.4 | 0.5 | 0.5 | 0.5 | 0.5 | 0.6 | 0.6 | 0.5 | 0.5 | 0.5 | 0.5 |
| 6 | SCV-07 10 mg/kg + | 0.5 | 0.5 | 0.5 | 0.5 | 0.5 | 0.5 | 0.5 | 0.5 | 0.5 | 0.5 | 0.6 | 0.6 | 0.5 | 0.5 | 0.6 | 0.6 | 0.4 | 0.4 | 0.5 | 0.5 |
| | CDDP 2 mg/kg | | | | | | | | | | | | | | | | | | | | |
| 7 | SCV-07 20 mg/kg + | 0.4 | 0.4 | 0.5 | 0.5 | 0.5 | 0.5 | 0.6 | 0.6 | 0.4 | 0.4 | 0.5 | 0.5 | 0.5 | 0.5 | 0.5 | 0.5 | 0.4 | 0.4 | 0.4 | 0.4 |
| | CDDP 2 mg/kg | | | | | | | | | | | | | | | | | | | | |

APPENDIX 3.6
Tumor volumes* (cm³) on Day 6

| Group | Treatment | F1 | F2 | F3 | F4 | F5 | M1 | M2 | M3 | M4 | M5 |
|---|---|---|---|---|---|---|---|---|---|---|---|
| 1 | Vehicle Control | 0.078732 | 0.055296 | 0.032 | 0.0625 | 0.0625 | 0.0744385 | 0.070304 | 0.0625 | 0.1026895 | 0.108 |
| 2 | CDDP 2 mg/kg | 0.0135 | 0.032 | 0.0135 | 0.004 | 0.0135 | 0.0135 | 0.0214375 | 0.0135 | 0.0135 | 0.0135 |
| 3 | CDDP 6 mg/kg | 0.002916 | 0 | 0.004 | 0.0078125 | 0 | 0.004 | 0.004 | 0.004 | 0.004 | 0.0135 |
| 4 | SCV-07 10 mg/kg | 0.0625 | 0.108 | 0.108 | 0.0625 | 0.108 | 0.108 | 0.0625 | 0.1715 | 0.108 | 0.0625 |
| 5 | SCV-07 20 mg/kg | 0.0625 | 0.0625 | 0.108 | 0.0625 | 0.032 | 0.0625 | 0.0625 | 0.108 | 0.0625 | 0.0625 |
| 6 | SCV-07 10 | 0.0625 | 0.0625 | 0.0625 | 0.0625 | 0.0625 | 0.108 | 0.0625 | 0.108 | 0.032 | 0.0625 |
| | mg/kg + | | | | | | | | | | |
| | CDDP 2 mg/kg | | | | | | | | | | |
| 7 | SCV-07 | 0.032 | 0.0625 | 0.0625 | 0.108 | 0.032 | 0.0625 | 0.0625 | 0.0625 | 0.032 | 0.032 |
| | 20 mg/kg + | | | | | | | | | | |
| | CDDP 2 mg/kg | | | | | | | | | | |

*Tumor volumes were calculated using the formula “Tumor Volume = Length × Width × Width/2” based on the data listed in Appendix 3.5.

APPENDIX 3.7
Tumor measurements (cm) on Day 8

| | | F1 | | F2 | | F3 | | F4 | | F5 | | M1 | | M2 | | M3 | | M4 | | M5 | |
|---|---|---|---|---|---|---|---|---|---|---|---|---|---|---|---|---|---|---|---|---|---|
| Group | Treatment | L | W | L | W | L | W | L | W | L | W | L | W | L | W | L | W | L | W | L | W |
| 1 | Vehicle | 1 | 1 | 0.9 | 0.9 | 0.8 | 0.8 | 0.9 | 0.9 | 1 | 1 | 1 | 1 | 1 | 1 | 1 | 1 | 1 | 1 | 1 | 1 |
| | Control | | | | | | | | | | | | | | | | | | | | |
| 2 | CDDP 2 mg/kg | 0.5 | 0.5 | 0.5 | 0.5 | 0.5 | 0.5 | 0.5 | 0.5 | 0.6 | 0.6 | 0.5 | 0.5 | 0.5 | 0.5 | 0.5 | 0.5 | 0.7 | 0.7 | 0.7 | 0.7 |
| 3 | CDDP 6 mg/kg | 0.3 | 0.3 | 0 | 0 | 0.3 | 0.3 | 0.3 | 0.3 | 0 | 0 | 0.5 | 0.5 | 0.5 | 0.5 | 0.5 | 0.5 | 0.5 | 0.5 | 0.5 | 0.5 |
| 4 | SCV-07 10 mg/kg | 0.7 | 0.7 | 1 | 0.9 | 1 | 1 | 0.8 | 0.8 | 0.87 | 0.87 | 1 | 1 | 0.8 | 0.8 | 0.9 | 0.9 | 0.8 | 0.8 | 0.8 | 0.8 |
| 5 | SCV-07 20 mg/kg | 0.8 | 0.8 | 0.9 | 0.9 | 0.8 | 0.8 | 0.89 | 0.89 | 0.6 | 0.6 | 0.9 | 0.9 | 0.7 | 0.7 | 0.9 | 0.9 | 0.7 | 0.7 | 0.87 | 0.87 |
| 6 | SCV-07 10 | 0.7 | 0.7 | 0.7 | 0.7 | 0.7 | 0.7 | 0.7 | 0.7 | 0.7 | 0.7 | 0.8 | 0.8 | 0.7 | 0.7 | 0.9 | 0.8 | 0.6 | 0.6 | 0.7 | 0.7 |
| | mg/kg + | | | | | | | | | | | | | | | | | | | | |
| | CDDP 2 mg/kg | | | | | | | | | | | | | | | | | | | | |
| 7 | SCV-07 20 | 0.6 | 0.6 | 0.7 | 0.7 | 0.7 | 0.7 | 0.8 | 0.8 | 0.6 | 0.6 | 0.7 | 0.7 | 0.7 | 0.7 | 0.7 | 0.7 | 0.6 | 0.6 | 0.7 | 0.7 |
| | mg/kg + | | | | | | | | | | | | | | | | | | | | |
| | CDDP 2 mg/kg | | | | | | | | | | | | | | | | | | | | |

APPENDIX 3.8
Tumor volumes* (cm³) on Day 8

| Group | Treatment | F1 | F2 | F3 | F4 | F5 | M1 | M2 | M3 | M4 | M5 |
|---|---|---|---|---|---|---|---|---|---|---|---|
| 1 | Vehicle | 0.5 | 0.3645 | 0.256 | 0.3645 | 0.5 | 0.5 | 0.5 | 0.5 | 0.5 | 0.5 |
| | Control | | | | | | | | | | |
| 2 | CDDP 2 mg/kg | 0.0625 | 0.0625 | 0.0625 | 0.0625 | 0.108 | 0.0625 | 0.0625 | 0.0625 | 0.1715 | 0.1715 |
| 3 | CDDP 6 mg/kg | 0.0135 | 0 | 0.0135 | 0.0135 | 0 | 0.0625 | 0.0625 | 0.0625 | 0.0625 | 0.0625 |
| 4 | SCV-07 10 mg/kg | 0.1715 | 0.3969 | 0.5 | 0.256 | 0.3292515 | 0.5 | 0.256 | 0.3645 | 0.256 | 0.256 |
| 5 | SCV-07 20 mg/kg | 0.256 | 0.3292515 | 0.256 | 0.3524845 | 0.108 | 0.3645 | 0.1715 | 0.3645 | 0.1715 | 0.3292515 |
| 6 | SCV-07 10 mg/kg + | 0.1715 | 0.1715 | 0.1715 | 0.1715 | 0.1715 | 0.256 | 0.1715 | 0.288 | 0.108 | 0.1715 |
| | CDDP 2 mg/kg | | | | | | | | | | |
| 7 | SCV-07 20 mg/kg + | 0.108 | 0.1715 | 0.1715 | 0.256 | 0.108 | 0.1715 | 0.1715 | 0.1715 | 0.108 | 0.1715 |
| | CDDP 2 mg/kg | | | | | | | | | | |

*Tumor volumes were calculated using the formula “Tumor Volume = Length × Width × Width/2” based on the data listed in Appendix 3.7.

APPENDIX 3.9
Tumor measurements (cm) on Day 10

| | | F1 | | F2 | | F3 | | F4 | | F5 | | M1 | | M2 | | M3 | | M4 | | M5 | |
|---|---|---|---|---|---|---|---|---|---|---|---|---|---|---|---|---|---|---|---|---|---|
| Group | Treatment | L | W | L | W | L | W | L | W | L | W | L | W | L | W | L | W | L | W | L | W |
| 1 | Vehicle | 1.2 | 1.2 | 1.1 | 1.1 | 1.2 | 1.3 | 1.4 | 1.4 | 1.3 | 1.5 | 1.25 | 1.25 | 1.4 | 1.4 | 1.3 | 1.3 | 1.4 | 1.4 | 1.3 | 1.3 |
| | Control | | | | | | | | | | | | | | | | | | | | |
| 2 | CDDP 2 | 0.6 | 0.6 | 0.7 | 0.7 | 0.7 | 0.7 | 0.8 | 0.8 | 0.7 | 0.7 | 0.7 | 0.7 | 0.7 | 0.7 | 0.8 | 0.8 | 0.9 | 0.9 | 0.9 | 0.9 |
| | mg/kg | | | | | | | | | | | | | | | | | | | | |
| 3 | CDDP 6 | 0.5 | 0.5 | 0 | 0 | 0.5 | 0.5 | 0.5 | 0.5 | 0 | 0 | 0.6 | 0.6 | 0.6 | 0.6 | 0.7 | 0.7 | 0.7 | 0.7 | 0.7 | 0.7 |
| | mg/kg | | | | | | | | | | | | | | | | | | | | |
| 4 | SCV-07 | 1 | 1 | 1.1 | 1.1 | 1.2 | 1.2 | 1.2 | 1.2 | 1.2 | 1.2 | 1.2 | 1.2 | 1 | 1 | 1.23 | 1.23 | 1 | 1 | 0.9 | 0.9 |
| | 10 mg/kg | | | | | | | | | | | | | | | | | | | | |
| 5 | SCV-07 | 1.09 | 1.11 | 1.2 | 1.1 | 1.05 | 1.05 | 1.1 | 1.1 | 0.8 | 0.8 | 1.19 | 1.2 | 0.9 | 0.9 | 1.1 | 1.1 | 1 | 1.2 | 1.1 | 1.23 |
| | 20 mg/kg | | | | | | | | | | | | | | | | | | | | |
| 6 | SCV-07 | 0.9 | 0.9 | 0.8 | 0.8 | 0.9 | 0.9 | 0.97 | 1.1 | 0.9 | 0.9 | 1 | 1 | 0.9 | 0.9 | 1.1 | 1.1 | 0.8 | 0.8 | 0.9 | 0.9 |
| | 10 mg/ | | | | | | | | | | | | | | | | | | | | |
| | kg + | | | | | | | | | | | | | | | | | | | | |
| | CDDP 2 | | | | | | | | | | | | | | | | | | | | |
| | mg/kg | | | | | | | | | | | | | | | | | | | | |
| 7 | SCV-07 | 0.9 | 1.1 | 0.9 | 0.9 | 1 | 1 | 1 | 1.12 | 0.8 | 0.8 | 0.9 | 0.9 | 0.9 | 0.99 | 0.9 | 0.9 | 0.85 | 0.85 | 0.9 | 0.9 |
| | 20 mg/ | | | | | | | | | | | | | | | | | | | | |
| | kg + | | | | | | | | | | | | | | | | | | | | |
| | CDDP | | | | | | | | | | | | | | | | | | | | |
| | 2 mg/kg | | | | | | | | | | | | | | | | | | | | |

APPENDIX 3.10
Tumor volumes* (cm³) on Day 10

| Group | Treatment | F1 | F2 | F3 | F4 | F5 | M1 | M2 | M3 | M4 | M5 |
|---|---|---|---|---|---|---|---|---|---|---|---|
| 1 | Vehicle | 0.864 | 0.6655 | 0.936 | 1.372 | 1.2675 | 0.9765625 | 1.372 | 1.0985 | 1.372 | 1.0985 |
| | Control | | | | | | | | | | |
| 2 | CDDP 2 mg/kg | 0.108 | 0.1715 | 0.1715 | 0.256 | 0.1715 | 0.1715 | 0.1715 | 0.256 | 0.3645 | 0.3645 |
| 3 | CDDP 6 mg/kg | 0.0625 | 0 | 0.0625 | 0.0625 | 0 | 0.108 | 0.108 | 0.1715 | 0.1715 | 0.1715 |
| 4 | SCV-07 | 0.5 | 0.702464 | 0.864 | 0.864 | 0.864 | 0.864 | 0.5 | 0.9304335 | 0.5 | 0.3645 |
| | 10 mg/kg | | | | | | | | | | |
| 5 | SCV-07 | 0.6593955 | 0.7018 | 0.5788125 | 0.6655 | 0.256 | 0.84966 | 0.3645 | 0.6655 | 0.72 | 0.74415 |
| | 20 mg/kg | | | | | | | | | | |
| 6 | SCV-07 10 | 0.3645 | 0.256 | 0.3645 | 0.517495 | 0.3645 | 0.5 | 0.3645 | 0.6655 | 0.256 | 0.3645 |
| | mg/kg + | | | | | | | | | | |
| | CDDP 2 mg/kg | | | | | | | | | | |
| 7 | SCV-07 20 | 0.4455 | 0.3645 | 0.5 | 0.56 | 0.256 | 0.3645 | 0.40095 | 0.3645 | 0.3070625 | 0.3645 |
| | mg/kg + | | | | | | | | | | |
| | CDDP 2 mg/kg | | | | | | | | | | |

*Tumor volumes were calculated using the formula “Tumor Volume = Length × Width × Width/2” based on the data listed in Appendix 3.9.

APPENDIX 3.11
Tumor measurements (cm) on Day 12

| | | F1 | | F2 | | F3 | | F4 | | F5 | | M1 | | M2 | | M3 | | M4 | | M5 | |
|---|---|---|---|---|---|---|---|---|---|---|---|---|---|---|---|---|---|---|---|---|---|
| Group | Treatment | L | W | L | W | L | W | L | W | L | W | L | W | L | W | L | W | L | W | L | W |
| 1 | Vehicle | 1.4 | 1.4 | 1.3 | 1.4 | 1.4 | 1.5 | 1.6 | 1.6 | 1.5 | 1.5 | 1.5 | 1.5 | 1.5 | 1.5 | 1.6 | 1.6 | 1.3 | 1.3 | 1.4 | 1.4 |
| | Control | | | | | | | | | | | | | | | | | | | | |
| 2 | CDDP 2 mg/kg | 1 | 1 | 1 | 1 | 1 | 1 | 1 | 1 | 0.9 | 0.9 | 0.9 | 0.9 | 0.9 | 0.9 | 1 | 1 | 1 | 1.1 | 1.1 | 1.1 |
| 3 | CDDP 6 mg/kg | 0.7 | 0.7 | 0.3 | 0.3 | 0.7 | 0.7 | 0.6 | 0.6 | 0.1 | 0.1 | 0.8 | 0.8 | 0.8 | 0.8 | 0.8 | 0.8. | 0.8 | 0.8 | 0.9 | 0.9 |
| 4 | SCV-07 | 1.25 | 1.2 | 1.4 | 1.4 | 1.5 | 1.5 | 1.3 | 1.3 | 1.25 | 1.5 | 1.3 | 1.3 | 1.2 | 1.2 | 1.36 | 1.36 | 1.2 | 1.2 | 1 | 1 |
| | 10 mg/kg | | | | | | | | | | | | | | | | | | | | |
| 5 | SCV-07 | 1.29 | 1.2 | 1.2 | 1.2 | 1.2 | 1.1 | 1.2 | 1.2 | 1 | 1 | 1.1 | 1.1 | 1.1 | 1.1 | 1.3 | 1.3 | 1.5 | 1.5 | 1.25 | 1.3 |
| | 20 mg/kg | | | | | | | | | | | | | | | | | | | | |
| 6 | SCV-07 | 1 | 1 | 0.9 | 0.9 | 1 | 1 | 1.2 | 1.2 | 1.1 | 1.1 | 1.2 | 1.2 | 1.1 | 1.1 | 1.2 | 1.2 | 1.07 | 1 | 1.1 | 1.1 |
| | 10 mg/kg + | | | | | | | | | | | | | | | | | | | | |
| | CDDP 2 mg/kg | | | | | | | | | | | | | | | | | | | | |
| 7 | SCV-07 20 | 1.1 | 1.2 | 1.1 | 1 | 1.3 | 1.3 | 1.2 | 1.21 | 0.9 | 1 | 1 | 1 | 1.1 | 1.1 | 1 | 1 | 1.1 | 1.1 | 1 | 1 |
| | mg/kg + | | | | | | | | | | | | | | | | | | | | |
| | CDDP 2 mg/kg | | | | | | | | | | | | | | | | | | | | |

APPENDIX 3.12
Tumor volumes* (cm³) on Day 12

| Group | Treatment | F1 | F2 | F3 | F4 | F5 | M1 | M2 | M3 | M4 | M5 |
|---|---|---|---|---|---|---|---|---|---|---|---|
| 1 | Vehicle | 1.372 | 1.183 | 1.47 | 2.048 | 1.6875 | 1.6875 | 1.6875 | 2.048 | 1.0985 | 1.372 |
| | Control | | | | | | | | | | |
| 2 | CDDP 2 mg/kg | 0.5 | 0.5 | 0.5 | 0.5 | 0.3645 | 0.3645 | 0.3645 | 0.5 | 0.55 | 0.6655 |
| 3 | CDDP 6 mg/kg | 0.1715 | 0.0135 | 0.1715 | 0.108 | 0.0005 | 0.256 | 0.256 | 0.256 | 0.256 | 0.3645 |
| 4 | SCV-07 10 mg/kg | 0.9 | 1.372 | 1.6875 | 1.0985 | 1.171875 | 1.0985 | 0.864 | 1.257728 | 0.864 | 0.5 |
| 5 | SCV-07 20 mg/kg | 0.9288 | 0.864 | 0.726 | 0.864 | 0.5 | 0.6655 | 0.6655 | 1.0985 | 1.6875 | 1.015625 |
| 6 | SCV-07 10 mg/kg + | 0.5 | 0.3645 | 0.5 | 0.864 | 0.6655 | 0.864 | 0.6655 | 0.864 | 0.535 | 0.6655 |
| | CDDP 2 mg/kg | | | | | | | | | | |
| 7 | SCV-07 20 mg/kg + | 0.726 | 0.55 | 1.10019 | 0.8712 | 0.405 | 0.5 | 0.6655 | 0.5 | 0.6655 | 0.5 |
| | CDDP 2 mg/kg | | | | | | | | | | |

*Tumor volumes were calculated using the formula “Tumor Volume = Length × Width × Width/2” based on the data listed in Appendix 11.

APPENDIX 3.13
Tumor measurements (cm) on Day 14

| | | F1 | | F2 | | F3 | | F4 | | F5 | | M1 | | M2 | | M3 | | M4 | | M5 | |
|---|---|---|---|---|---|---|---|---|---|---|---|---|---|---|---|---|---|---|---|---|---|
| Group | Treatment | L | W | L | W | L | W | L | W | L | W | L | W | L | W | L | W | L | W | L | W |
| 1 | Vehicle | 1.7 | 1.7 | 1.6 | 1.6 | 1.6 | 1.7 | 1.9 | 1.9 | 1.9 | 1.8 | 1.6 | 1.6 | 1.7 | 1.7 | 1.6 | 1.6 | 1.5 | 1.5 | 1.7 | 1.7 |
| | Control | | | | | | | | | | | | | | | | | | | | |
| 2 | CDDP 2 mg/kg | 1.2 | 1.2 | 1.2 | 1.2 | 1.1 | 1.1 | 1.18 | 1.2 | 1.1 | 1.1 | 1.2 | 1.2 | 1.1 | 1.2 | 1.2 | 1.2 | 1.1 | 1.3 | 1.21 | 1.21 |
| 3 | CDDP 6 mg/kg | 0.8 | 0.8 | 0.6 | 0.6 | 0.8 | 0.8 | 0.8 | 0.8 | 0.4 | 0.4 | 1.1 | 1.1 | 1.1 | 1.1 | 1 | 1 | 1 | 1 | 1 | 1 |
| 4 | SCV-07 10 mg/kg | 1.4 | 1.4 | 1.5 | 1.5 | 1.6 | 1.7 | 1.5 | 1.5 | 1.38 | 1.4 | 1.55 | 1.5 | 1.36 | 1.36 | 1.4 | 1.4 | 1.33 | 1.33 | 1.2 | 1.2 |
| 5 | SCV-07 20 mg/kg | 1.3 | 1.3 | 1.4 | 1.4 | 1.3 | 1.3 | 1.4 | 1.4 | 1.2 | 1.2 | 1.3 | 1.3 | 1.3 | 1.3 | 1.5 | 1.5 | 1.5 | 1.6 | 1.3 | 1.3 |
| 6 | SCV-07 10 mg/ | 1.2 | 1.2 | 1 | 1 | 1.2 | 1.2 | 1.4 | 1.4 | 1.3 | 1.3 | 1.4 | 1.4 | 1.3 | 1.3 | 1.4 | 1.4 | 1.2 | 1.2 | 1.2 | 1.2 |
| | kg + CDDP 2 mg/kg | | | | | | | | | | | | | | | | | | | | |
| 7 | SCV-07 20 mg/kg + | 1.3 | 1.4 | 1.2 | 1.2 | 1.4 | 1.4 | 1.3 | 1.4 | 1 | 1.1 | 1.1 | 1.2 | 1.2 | 1.2 | 1.2 | 1 | 1.2 | 1.2 | 1.2 | 1.2 |
| | CDDP 2 mg/kg | | | | | | | | | | | | | | | | | | | | |

APPENDIX 3.14
Tumor volumes* (cm³) on Day 14

| Group | Treatment | F1 | F2 | F3 | F4 | F5 | M1 | M2 | M3 | M4 | M5 |
|---|---|---|---|---|---|---|---|---|---|---|---|
| 1 | Vehicle | 2.4565 | 2.048 | 2.176 | 3.4295 | 3.078 | 2.048 | 2.4565 | 2.048 | 1.6875 | 2.4565 |
| | Control | | | | | | | | | | |
| 2 | CDDP 2 mg/kg | 0.864 | 0.864 | 0.6655 | 0.83544 | 0.6655 | 0.8596854 | 0.726 | 0.864 | 0.7865 | 0.8857805 |
| 3 | CDDP 6 mg/kg | 0.256 | 0.108 | 0.256 | 0.256 | 0.032 | 0.6655 | 0.6655 | 0.5 | 0.5 | 0.5 |
| 4 | SCV-07 10 mg/kg | 1.372 | 1.6875 | 2.176 | 1.6875 | 1.33308 | 1.74375 | 1.257728 | 1.372 | 1.1763185 | 0.864 |
| 5 | SCV-07 20 mg/kg | 1.0985 | 1.372 | 1.0985 | 1.372 | 0.864 | 1.0985 | 1.0985 | 1.6875 | 1.8 | 1.0985 |
| 6 | SCV-07 10 mg/kg + | 0.864 | 0.5 | 0.864 | 1.372 | 1.0985 | 1.372 | 1.0985 | 1.372 | 0.864 | 0.864 |
| | CDDP 2 mg/kg | | | | | | | | | | |
| 7 | SCV-07 20 mg/kg + | 1.183 | 0.864 | 1.372 | 1.183 | 0.55 | 0.726 | 0.864 | 0.6 | 0.864 | 0.864 |
| | CDDP 2 mg/kg | | | | | | | | | | |

*Tumor volumes were calculated using the formula “Tumor Volume = Length × Width × Width/2” based on the data listed in Appendix 3.13.

APPENDIX 3.15
Tumor weights (g) on Day 16

| Group | Treatment | F1 | F2 | F3 | F4 | F5 | M1 | M2 | M3 | M4 | M5 |
|---|---|---|---|---|---|---|---|---|---|---|---|
| 1 | Vehicle | 4.75 | 5.1 | 3.9 | 4.3 | 2.98 | 3.83 | 4.6 | 3.68 | 3.73 | 4 |
| | Control | | | | | | | | | | |
| 2 | CDDP 2 mg/kg | 1.56 | 0.7 | 1.3 | 1.9 | 1.68 | 1.89 | 1.6 | 2 | 2.3 | 1.82 |
| 3 | CDDP 6 mg/kg | 0.3 | 0.5 | 0.9 | 0.4 | 0.78 | 1.1 | 1.2 | 1.9 | 1.2 | 0.9 |
| 4 | SCV-07 10 mg/kg | 3.38 | 4.1 | 4.4 | 3.9 | 3 | 3.4 | 3.02 | 3.27 | 3 | 2.49 |
| 5 | SCV-07 20 mg/kg | 2.54 | 3.3 | 2 | 3.2 | 2.49 | 2.55 | 2.46 | 1.85 | 3 | 2.18 |
| 6 | SCV-07 10 mg/kg + | 2 | 3.2 | 1.8 | 2.5 | 2 | 3.14 | 2.32 | 2.11 | 2.85 | 2.21 |
| | CDDP 2 mg/kg | | | | | | | | | | |
| 7 | SCV-07 20 mg/kg + | 1.62 | 2.4 | 2.7 | 2.7 | 1.48 | 1.59 | 1.32 | 1.23 | 1.47 | 1.42 |
| | CDDP 2 mg/kg | | | | | | | | | | |

APPENDIX 3.16
Body weights (g) on Day 0

| Group | Treatment | F1 | F2 | F3 | F4 | F5 | M1 | M2 | M3 | M4 | M5 |
|---|---|---|---|---|---|---|---|---|---|---|---|
| 1 | Vehicle | 17.4 | 18.2 | 16 | 16.7 | 16.8 | 18.2 | 18 | 18.5 | 17.2 | 17 |
| | Control | | | | | | | | | | |
| 2 | CDDP 2 mg/kg | 17.6 | 17.2 | 17.5 | 16.3 | 15.7 | 17.5 | 18.2 | 18.5 | 18.7 | 18 |
| 3 | CDDP 6 mg/kg | 17.9 | 16.2 | 17.5 | 17 | 16 | 18.1 | 18 | 18.7 | 18.3 | 18 |
| 4 | SCV-07 10 mg/kg | 17 | 16.7 | 17 | 17.3 | 16.3 | 18.4 | 17.9 | 18 | 17 | 18.3 |
| 5 | SCV-07 20 mg/kg | 16 | 17.2 | 17.3 | 17.5 | 16.7 | 17.8 | 16.8 | 17.1 | 17.3 | 17.8 |
| 6 | SCV-07 10 mg/kg + | 17.5 | 16.5 | 16 | 16 | 16.6 | 18.5 | 18 | 17.3 | 18.2 | 16.4 |
| | CDDP 2 mg/kg | | | | | | | | | | |
| 7 | SCV-07 20 mg/kg + | 16 | 17.2 | 16.4 | 16.5 | 16.8 | 16.7 | 17.2 | 17.6 | 18 | 17.3 |
| | CDDP 2 mg/kg | | | | | | | | | | |

APPENDIX 3.17
Body weights (g) on Day 3

| Group | Treatment | F1 | F2 | F3 | F4 | F5 | M1 | M2 | M3 | M4 | M5 |
|---|---|---|---|---|---|---|---|---|---|---|---|
| 1 | Vehicle | 17.6 | 18.4 | 16 | 16.9 | 17 | 18.4 | 18 | 18.8 | 17.4 | 17.2 |
| | Control | | | | | | | | | | |
| 2 | CDDP 2 mg/kg | 15 | 16.2 | 16.6 | 14 | 15.2 | 17.1 | 18.4 | 18.3 | 17.6 | 17.2 |
| 3 | CDDP 6 mg/kg | 14 | 14.1 | 15.5 | 15 | 14 | 17.2 | 16.9 | 17.5 | 17.9 | 17.2 |
| 4 | SCV-07 10 mg/kg | 17.6 | 16.8 | 16.8 | 18 | 16.7 | 18.6 | 18.2 | 18.3 | 17.2 | 18.5 |
| 5 | SCV-07 20 mg/kg | 17.4 | 17.6 | 17.5 | 17.9 | 17 | 18 | 17 | 17.3 | 17.5 | 18 |
| 6 | SCV-07 10 mg/kg + | 17.1 | 15 | 16.3 | 15 | 14.7 | 18.3 | 17.5 | 17.6 | 18 | 17 |
| | CDDP 2 mg/kg | | | | | | | | | | |
| 7 | SCV-07 20 mg/kg + | 15 | 17.1 | 15.2 | 15.4 | 16 | 15.1 | 16.4 | 16.4 | 17.4 | 17.1 |
| | CDDP 2 mg/kg | | | | | | | | | | |

APPENDIX 3.18
Body weights (g) on Day 6

| Group | Treatment | F1 | F2 | F3 | F4 | F5 | M1 | M2 | M3 | M4 | M5 |
|---|---|---|---|---|---|---|---|---|---|---|---|
| 1 | Vehicle | 18.3 | 19 | 16.5 | 17.8 | 18.5 | 18.6 | 18.2 | 19 | 17.6 | 17.4 |
| | Control | | | | | | | | | | |
| 2 | CDDP 2 mg/kg | 16.4 | 16.8 | 17.7 | 15.3 | 16.4 | 18.7 | 19 | 19 | 18.3 | 17.6 |
| 3 | CDDP 6 mg/kg | 15 | 15.2 | 16 | 16.4 | 15 | 17.9 | 15.3 | 17.4 | 16 | 17.8 |
| 4 | SCV-07 10 mg/kg | 18 | 17.4 | 18.2 | 18.4 | 17.1 | 18.8 | 18.4 | 18.7 | 17.4 | 18.8 |
| 5 | SCV-07 20 mg/kg | 17.9 | 18.5 | 17.7 | 18.3 | 17.4 | 18.3 | 17.2 | 17.5 | 17.7 | 18.3 |
| 6 | SCV-07 10 mg/kg + | 18 | 16 | 17.3 | 16.1 | 16.3 | 19 | 19 | 18.6 | 18.5 | 16.3 |
| | CDDP 2 mg/kg | | | | | | | | | | |
| 7 | SCV-07 20 mg/kg + | 16.4 | 18.4 | 16.6 | 16.6 | 17.1 | 16 | 17 | 16 | 18.9 | 16 |
| | CDDP 2 mg/kg | | | | | | | | | | |

APPENDIX 3.19
Body weights (g) on Day 9

| Group | Treatment | F1 | F2 | F3 | F4 | F5 | M1 | M2 | M3 | M4 | M5 |
|---|---|---|---|---|---|---|---|---|---|---|---|
| 1 | Vehicle | 19.1 | 19.2 | 16.7 | 17.8 | 18.2 | 19.2 | 18.5 | 19.3 | 18.4 | 17.6 |
| | Control | | | | | | | | | | |
| 2 | CDDP 2 mg/kg | 16.8 | 16.8 | 18 | 16 | 17 | 18.9 | 19.1 | 19 | 18.4 | 17.4 |
| 3 | CDDP 6 mg/kg | 15 | 15 | 15.5 | 16.2 | 15 | 16.4 | 15.2 | 17.3 | 16 | 15.7 |
| 4 | SCV-07 10 mg/kg | 19 | 18.5 | 18.6 | 18.8 | 17.1 | 19.1 | 18.8 | 19 | 18 | 19 |
| 5 | SCV-07 20 mg/kg | 18 | 18.5 | 17.1 | 18.9 | 18.7 | 18.7 | 17.7 | 18.1 | 18.1 | 18.6 |
| 6 | SCV-07 10 mg/kg + | 18.2 | 16.5 | 17.3 | 16.3 | 16.8 | 19 | 18.9 | 15 | 15 | 15 |
| | CDDP 2 mg/kg | | | | | | | | | | |
| 7 | SCV-07 20 mg/kg + | 16 | 17.1 | 16.4 | 16 | 17.4 | 15.7 | 17 | 15.8 | 15 | 16.6 |
| | CDDP 2 mg/kg | | | | | | | | | | |

APPENDIX 3.20
Body weights (g) on Day 12

| Group | Treatment | F1 | F2 | F3 | F4 | F5 | M1 | M2 | M3 | M4 | M5 |
|---|---|---|---|---|---|---|---|---|---|---|---|
| 1 | Vehicle | 19.4 | 20 | 17 | 19.2 | 16.7 | 20.4 | 20.5 | 21.2 | 18.4 | 19.6 |
| | Control | | | | | | | | | | |
| 2 | CDDP 2 mg/kg | 17.1 | 15.1 | 17.5 | 16.3 | 18 | 18.3 | 20 | 20 | 21 | 19 |
| 3 | CDDP 6 mg/kg | 13.1 | 17 | 17 | 16 | 16 | 18.9 | 16 | 16.1 | 18.3 | 17.8 |
| 4 | SCV-07 10 mg/kg | 19.5 | 19 | 19.8 | 18.8 | 16.6 | 20 | 19 | 17.7 | 19.7 | 18.8 |
| 5 | SCV-07 20 mg/kg | 18.5 | 18.6 | 17.8 | 19.2 | 20 | 18 | 20 | 19.7 | 19.3 | 20.1 |
| 6 | SCV-07 10 mg/kg + | 18.3 | 16.7 | 17.5 | 16.9 | 19.8 | 21.4 | 20 | 16.8 | 20 | 15.5 |
| | CDDP 2 mg/kg | | | | | | | | | | |
| 7 | SCV-07 20 mg/kg + | 16.1 | 17.4 | 17.6 | 17.2 | 19 | 17.9 | 20.6 | 17.9 | 18.2 | 17.7 |
| | CDDP 2 mg/kg | | | | | | | | | | |

APPENDIX 3.21
Body weights (g) on Day 15

| Group | Treatment | F1 | F2 | F3 | F4 | F5 | M1 | M2 | M3 | M4 | M5 |
|---|---|---|---|---|---|---|---|---|---|---|---|
| 1 | Vehicle | 19.8 | 21 | 18.3 | 20.5 | 18.3 | 21.2 | 21.2 | 21.4 | 18.9 | 20 |
| | Control | | | | | | | | | | |
| 2 | CDDP 2 mg/kg | 15.2 | 15 | 17.6 | 16.8 | 17.5 | 17.1 | 19.3 | 18.5 | 20 | 18.5 |
| 3 | CDDP 6 mg/kg | 11.1 | 16.3 | 14.4 | 16.3 | 13.6 | 18.4 | 14.6 | 14.7 | 15.8 | 16.5 |
| 4 | SCV-07 10 mg/kg | 20.3 | 18.6 | 19.1 | 18.9 | 16 | 20.2 | 19.4 | 16.7 | 20 | 19.6 |
| 5 | SCV-07 20 mg/kg | 19 | 18.8 | 19.1 | 18.1 | 19.8 | 18.3 | 20.5 | 18.9 | 20 | 20.6 |
| 6 | SCV-07 10 mg/kg + | 18.1 | 16.4 | 17 | 15.8 | 19.1 | 21.4 | 20.1 | 16.8 | 19.4 | 15.6 |
| | CDDP 2 mg/kg | | | | | | | | | | |
| 7 | SCV-07 20 mg/kg + | 15.8 | 16.4 | 17.6 | 17.4 | 18.8 | 16.3 | 17.9 | 15.7 | 18.8 | 16.7 |
| | CDDP 2 mg/kg | | | | | | | | | | |

### Example 4

### In Vitro Effects of SCV-07 on Proliferation of B16, LLC, and RenCa Cell Lines

### Abbreviations

5-Fu 5-Fluorouracil

CV Coefficient of Variation

DMEM Dulbecco's modified Eagle's medium

DMF N,N-dimethyl formamide

DTIC 5-(3,3-Dimethyl-1-triazenyl)imidazole-4-carboxamide

FBS Fetal bovine serum

LLC Lewis lung carcinoma

MTT Methylthiazolyldiphenyl-tetrazolium bromide

NA Not applicable

OD Optical density

PBS Phosphate buffered saline

RPMI Roswell Park Memorial Institute

SD Standard Deviation

SDS Sodium dodecyl sulphate

SOP Standard operation procedure

VBI Vital Bridge (China), Inc.

### Vs Versus

### Summary

The study was undertaken to evaluate the in vitro cytotoxic effect of SCV-07 on B16, LLC, and RenCa cells.

B16, LLC, or RenCa cells were cultured in 96-well plates in the presence of SCV-07 or a positive control drug (i.e., DTIC, 5-Fu, and Cisplatin) at 12 different concentrations including the blank control. The concentrations of SCV-07 were chosen based on the plasma concentration approximated from the efficacious dose of the previous in vivo studies. The concentrations of 5-Fu and Cisplatin were selected per their respective IC₅₀ values reported in the literature. The incubation time of SCV-07 and positive control drugs varied from 24 to 72 hours. The inhibitory effects of the drugs on cell proliferation were determined by the MTT method.

The treatment of 5-Fu and Cisplatin resulted in significant cytotoxic effects in the corresponding cell lines. The IC₅₀ values for 5-Fu to inhibit B16 cell proliferation were estimated to be 0.26, 0.38, and 0.26 μg/mL in three assays. In RenCa cells, the IC₅₀ values for 5-Fu were estimated to be 0.03, 0.04, and 0.04 μg/mL in three assays. The IC₅₀ values for Cisplatin to inhibit LLC cell proliferation were estimated to be 3.26, 3.07, and 3.10 μg/mL in three assays. SCV-07 at all test concentrations (0.05 to 100 μg/mL) did not inhibit cell proliferation in the cultured B16, LLC, and RenCa cells. The IC₅₀ values for SCV-07 were not obtained due to the lack of fit of its concentration-inhibition curves.

In conclusion, this study demonstrated that SCV-07 did not have in vitro cytotoxic effects on cultured B16, LLC, and RenCa tumor cells.

### Introduction

The objective of the study was to evaluate in vitro cytotoxic effects of SCV-07 on B16, LLC, and RenCa cells.

SCV-07 is an immunomodulator. It has been demonstrated in the previous in vivo studies to inhibit the growth of tumor cells (B16, LLC, or RenCa) subcutaneously implanted in mice⁽¹⁻³⁾. In this study, the in vitro cytotoxic effects of SCV-07 on these tumor cell lines were evaluated.

B16, LLC, and RenCa cells were cultured in 96-well plates in the presence of SCV-07 or a positive control drug (DTIC, 5-Fu, or Cisplatin). The incubation time of the drugs in different cell lines varied from 24 to 72 hours. The MTT assay was used for the assessment of the inhibition of cell proliferation.

### Materials and Methods

### Materials

### SCV-07

SCV-07 (Lot # RR002101) was provided by the Sponsor. A stock solution of SCV-07 at the concentration of 0.5 mg/mL was prepared by dissolving 4.2 mg SCV-07 in 8.4 mL of sterile Dulbecco's PBS (Invitrogen, Cat# 14190-144). The stock solution was then sterile filtered, stored at 2-8° C., and protected from light with tin foil. Prior to use, the stock solution was further diluted to various concentrations with culture media.

### DTIC

DTIC was purchased from Sigma (Cat. # D2390, Lot # 026K1363). A stock solution of 10 mg/mL was prepared by dissolving 8.8 mg of DTIC in 500 μL of 0.1 N HCl, followed by the addition of 380 μL of Milli-Q water. Once prepared, the stock solution was sterile filtered, stored at 2-8° C., and protected from light with tin foil. Prior to use, the stock solution was further diluted to various concentrations with culture media.

### 5-FU

5-Fu was purchased from Sigma (Cat. # F6627, Lot # 125K1499). A stock solution of 0.5 mg/mL was prepared by dissolving 4.8 mg of 5-Fu in 9.6 mL of sterile Dulbecco's PBS (Invitrogen, Cat. # 14190-144). Once prepared, the stock solution was sterile filtered, stored at 2-8° C., and protected from light with tin foil. Prior to use, the stock solution was further diluted to various concentrations with culture media.

### Cisplatin

Cisplatin was purchased from Qilu Pharmaceutical Co. LTD. A stock solution of 1 mg/mL was prepared by dissolving 10 mg of cisplatin in 10 mL of sterile Dulbecco's PBS (Invitrogen, Cat. # 14190-144). Once prepared, the stock solution was sterile filtered and stored at 2-8° C. Prior to use, the stock solution was further diluted to various concentrations with culture media.

### Other Materials

MTT was purchased from Sigma (Cat. # M2128). FBS, Penicillin-Streptomycin, DMEM and RPMI-1640 media were purchased from Invitrogen. Falcon® 96-well flat-bottom plates (BD, Cat. # 353072) were purchased from Fisher Scientific.

### Test Systems

### B16 Cell Culture

B16 melanoma cell line was obtained from Shanghai Cell Bank, Chinese Academy of Sciences. The cells were cultured in RPMI-1640 medium supplemented with 10% FBS, 100 units/mL Penicillin, and 100 μg/mL Streptomycin.

### LLC Cell Culture

LLC cell line was obtained from Shanghai Cell Bank, Chinese Academy of Sciences. The cells were cultured in DMEM supplemented with 10% FBS, 100 units/mL Penicillin, and 100 μg/mL Streptomycin.

### RenCa Cell Culture

RenCa cell line was obtained from Chinese Military Academy of Sciences. The cells were cultured in RPMI-1640 medium supplemented with 10% FBS, 100 units/mL Penicillin, and 100 μg/mL Streptomycin.

### MTT Assay

MTT assay was conducted according to VBI SOP 65.026. Briefly, the suspensions of B16, LLC, or RenCa cells were prepared with the corresponding culture media described above. One hundred μL of the cell suspensions were seeded into each well of Falcon® 96-well flat-bottom plates. The seeding density was 10000 cells per well (for B16 and LLC cell lines) or 7000 cells per well (for RenCa cell line). Drug treatment was performed by adding 25 μL of drugs to the plates and then incubating the plates at 37° C. with 5% CO₂ for a pre-defined period of time (see Table 4.1 for details on the drug treatment). The drugs were treated with 12 concentrations including the blank control, with each concentration tested in quadruplicates. The concentrations of SCV-07 were chosen based on the plasma concentrations approximated from the efficacious dose of the previous in vivo studies. The concentrations of 5-Fu and Cisplatin were selected per their respective IC₅₀ values reported in the literature for the corresponding cell lines. After the drug treatment, MTT was added to each well at a final concentration of 1 mg/mL, and the cell incubation was continued for 4 hours. At the end of MTT incubation, the extraction buffer consisting of SDS and DMF was added to the plates to solubilize formazan converted from MTT by viable cells. OD of each well was then measured with Tecan Infinite M200 plate reader at 570 nm.

TABLE 4.1
Drug Treatment Design

| Cell | | Duration | | | | | | | | | | | | |
|---|---|---|---|---|---|---|---|---|---|---|---|---|---|---|
| Line | Drug | (hours) | Final Drug Concentration (μg/mL) | | | | | | | | | | | |
| B16 | SCV-07 | 24 | 0 | 0.05 | 0.1 | 0.2 | 0.5 | 1 | 2 | 5 | 10 | 20 | 50 | 100 |
| | DTIC | 24 | 0 | 0.25 | 0.5 | 1 | 2.5 | 5 | 10 | 25 | 50 | 100 | 250 | 500 |
| | 5-Fu | 24 | 0 | 0.05 | 0.1 | 0.2 | 0.5 | 1 | 2 | 5 | 10 | 20 | 50 | 100 |
| LLC | SCV-07 | 48 | 0 | 0.05 | 0.1 | 0.2 | 0.5 | 1 | 2 | 5 | 10 | 20 | 50 | 100 |
| | Cisplatin | 48 | 0 | 0.1 | 0.2 | 0.5 | 1 | 2 | 5 | 10 | 20 | 50 | 100 | 200 |
| RenCa | SCV-07 | 72 | 0 | 0.05 | 0.1 | 0.2 | 0.5 | 1 | 2 | 5 | 10 | 20 | 50 | 100 |
| | 5-Fu | 72 | 0 | 0.05 | 0.1 | 0.2 | 0.5 | 1 | 2 | 5 | 10 | 20 | 50 | 100 |

### Data Analyses

### Calculation of Mean and SD

Raw OD data were imported into Microsoft Excel for the calculation of mean and

SD.

### Calculation of IC₅₀

The IC₅₀ for inhibiting cell proliferation was calculated with Prism 5.01 (GraphPad Software, Inc.). The concentrations of a drug that resulted in cell killing were excluded from determination of the IC₅₀. The IC₅₀ was estimated by using the formula below:

$$Y = Bottom + \frac{Top - Bottom}{1 + {10}^{( X - Log IC 50 )}}$$

Herein, X stands for concentration of a drug, and Y for corresponding OD. Bottom stands for theoretically lowest OD (corresponding to the maximal inhibition of cell growth), while Top represents the theoretical highest OD. The IC₅₀ represents the concentration of the drug producing 50% response. Upon entering all X and Y values, the values of Bottom, Top, and IC₅₀ were automatically determined via the program by fitting to the built-in inhibition model (i.e., Log [Inhibitor] vs Response model).

### Results

### Effects of SCV-07 on B16 Cells

The effects of SCV-07 and positive control drugs on B16 cell proliferation were measured. The calculated IC₅₀ values are listed in Table 4.2. Raw data and the calculated mean and SD are tabulated in Appendices 4.1-4.4. The concentration-inhibition curve of SCV-07 was essentially flat, indicating the absence of a cytotoxic effect (i.e., inhibition of cell proliferation) of SCV-07 on B16 cells. DTIC was initially used as the positive control. However, the cytotoxic effect was noted only at higher concentrations (i.e., 250 and 500 μg/mL), and IC₅₀ was not established due to the failure of the curve to converge. This is probably due to the lack of hepatocyte-dependent activation of DTIC. The inhibition of cell growth was observed for 5-Fu at a range of concentrations from 0.2 to 20 μg/mL. Cell killing was induced at 50 and 100 μg/mL. The two concentrations were thereby excluded from the IC₅₀ analysis. The IC₅₀ values were determined to be 0.26, 0.38, and 0.26 μg/mL for 5-Fu in three assays. On the contrary, IC₅₀ values were not obtained for SCV-07 due to the lack of fit of its concentration-inhibition curves.

TABLE 4.2
IC₅₀ for Growth Inhibition of B16 Cells

| | IC₅₀ (μg/mL) | | |
|---|---|---|---|
| Assay ID | SCV-07 | DTIC | 5-Fu |
| 11280701 | — | — | NT |
| 12050701 | — | NT | 0.26 |
| 12050702 | — | NT | 0.38 |
| 12050703 | — | NT | 0.26 |

“—” indicates that IC₅₀ value was not obtained due to lack of fit.
NT: not tested

### Effects of SCV-07 on LLC Cells

The effects of SCV-07 and Cisplatin (positive control drug) on LLC cell proliferation were measured. The calculated values of IC₅₀ are listed in Table 4.3. Raw data and the calculated mean and SD are tabulated in Appendices 4.5-4.7. The concentration-inhibition curve of SCV-07 was essentially flat, indicating the absence of the cytotoxicity in LLC cells. On the contrary, the cytotoxic effect was observed for Cisplatin at concentrations of 1.0 μg/mL and above. The IC₅₀ values for Cisplatin were 3.26, 3.07, and 3.10 μg/mL in three assays, while IC₅₀ values could not be established for SCV-07 due to the lack of fit of its concentration-inhibition curves.

TABLE 4.3
IC₅₀ for Growth Inhibition of LLC Cells

IC₅₀ (μg/mL)

| Assay ID | SCV-07 | Cisplatin |
|---|---|---|
| 12060704 | — | 3.26 |
| 12060705 | — | 3.07 |
| 12060706 | — | 3.10 |

“—” indicates that IC50 value was not obtained due to lack of fit.

### Effects of SCV-07 on RenCa Cells

The effects of SCV-07 and 5-Fu (positive control drug) on RenCa cell proliferation were measured. The calculated IC₅₀ values are listed in Table 4.4. Raw data and the calculated mean and SD are tabulated in Appendices 4.8-4.10. The concentration-inhibition curve of SCV-07 was essentially flat, indicating the absence of the cytotoxicity in RenCa cells. On the contrary, the cytotoxic effects were observed for 5-Fu at a range of concentrations from 0.05 to 10 μg/mL. Cell killing was induced at 20, 50, and 100 μg/mL. The three concentrations were thereby excluded from the IC₅₀ analysis. The IC₅₀ values for 5-Fu were 0.03, 0.04, and 0.04 μg/mL in three assays. By contrast, the IC₅₀ values were not obtained for SCV-07 due to the lack of fit of its concentration-inhibition curves.

TABLE 4.4
IC₅₀ for Growth Inhibition of RenCa Cells

IC₅₀ (μg/mL)

| Assay ID | SCV-07 | 5-Fu |
|---|---|---|
| 12070704 | — | 0.03 |
| 12070705 | — | 0.04 |
| 12070706 | — | 0.04 |

“—” indicates that IC₅₀ value was not obtained due to lack of fit.

### Conclusion and Discussion

The treatment of 5-Fu and Cisplatin resulted in significant inhibition of cell proliferation in the corresponding cell lines, validating this assay for use in determining the potential cytotoxicity of the test compounds. The IC₅₀ values for 5-Fu to inhibit B16 cell proliferation were estimated to be 0.26, 0.38, and 0.26 μg/mL in three assays. In RenCa cells, the IC₅₀ values for 5-Fu were estimated to be 0.03, 0.04, and 0.04 μg/mL in three assays. The IC₅₀ values for Cisplatin to inhibit LLC cell proliferation were estimated to be 3.26, 3.07, and 3.10 μg/mL in three assays. The cytotoxicity was noted for DTIC at higher concentrations (i.e., 250 and 500 μg/mL). The lack of appreciable cytotoxicity at lower concentrations of DTIC is consistent with the requirement of metabolic conversion of DTIC to more toxic metabolites by hepatocytes, which were not included in the assay.

In sharp contrast to significant cytotoxicity by positive control drugs, SCV-07 did not result in the inhibition of cell proliferation in the cultured B16, LLC, or RenCa cells. The IC₅₀ values for SCV-07 were not obtained due to the lack of fit of its concentration-inhibition curves.

The absence of a cytotoxic effect of SCV-07 is in agreement with the mechanism of action for a typical immunomodulator which exerts its effects though activation of the immune system. However, it should be noted that cytotoxic effects of metabolites derived from SCV-07 remain to be determined. Like DTIC, hepatocytes-mediated metabolic activation of SCV-07 might be a prerequisite for a cytotoxic effect. Studies with SCV-07 metabolite(s) or with a cell culture system consisting of hepatocytes and tumor cells may help further define the role of cytotoxicity in the mechanism of action of SCV-07 for tumor therapy.

In conclusion, this study demonstrated that SCV-07 did not have in vitro cytotoxic effects on cultured B16, LLC, and RenCa tumor cells under the present experimental conditions.

APPENDIX 4.1
Raw Data and Calculated Mean and SD of MTT Assay 11280701
Cell line B16 (10000 cells/well)
Drug treatment (SCV-07 vs DTIC)
Drug treatment time 24 hours

| | | | | | | | |
|---|---|---|---|---|---|---|---|
| Conc. | | | | | | | |
| (ug/mL) | OD1 | OD2 | OD3 | OD4 | Mean | SD | CV |

SCV-07

| | | | | | | | |
|---|---|---|---|---|---|---|---|
| 0 | 1.0873 | 1.1178 | 1.1631 | 1.2261 | 1.15 | 0.060 | 5.25% |
| 0.05 | 1.1569 | 1.1254 | 1.1413 | 1.1492 | 1.14 | 0.013 | 1.18% |
| 0.1 | 1.176 | 1.1741 | 1.1855 | 1.085 | 1.16 | 0.047 | 4.07% |
| 0.2 | 1.1172 | 1.1665 | 1.1437 | 1.1428 | 1.14 | 0.020 | 1.76% |
| 0.5 | 1.1495 | 1.0958 | 1.0733 | 1.1737 | 1.12 | 0.046 | 4.14% |
| 1 | 1.1579 | 1.2365 | 1.1515 | 1.2437 | 1.20 | 0.049 | 4.13% |
| 2 | 1.2077 | 1.198 | 1.1919 | 1.2058 | 1.20 | 0.007 | 0.61% |
| 5 | 1.211 | 1.1803 | 1.1288 | 1.1662 | 1.17 | 0.034 | 2.91% |
| 10 | 1.1513 | 1.1447 | 1.137 | 1.2029 | 1.16 | 0.030 | 2.58% |
| 20 | 1.1496 | 1.2003 | 1.0976 | 1.148 | 1.15 | 0.042 | 3.65% |
| 50 | 1.0027 | 1.1377 | 1.1044 | 1.1857 | 1.11 | 0.078 | 7.00% |
| 100 | 1.0813 | 1.2025 | 1.0823 | 1.1933 | 1.14 | 0.067 | 5.89% |

DTIC

| | | | | | | | |
|---|---|---|---|---|---|---|---|
| 0 | 1.1309 | 1.2231 | 1.2149 | 1.0987 | 1.17 | 0.062 | 5.28% |
| 0.25 | 1.1613 | 1.1378 | 1.0642 | 1.0816 | 1.11 | 0.046 | 4.12% |
| 0.5 | 1.2172 | 1.1962 | 1.0476 | 1.0961 | 1.14 | 0.081 | 7.09% |
| 1 | 1.1808 | 1.2467 | 1.1306 | 1.1605 | 1.18 | 0.049 | 4.17% |
| 2.5 | 1.1187 | 1.1669 | 1.2015 | 0.9904 | 1.12 | 0.092 | 8.26% |
| 5 | 1.2193 | 1.2789 | 1.2814 | 1.1601 | 1.23 | 0.058 | 4.66% |
| 10 | 1.1866 | 1.163 | 1.2257 | 1.1542 | 1.18 | 0.032 | 2.70% |
| 25 | 1.1756 | 1.138 | 1.2473 | 1.1683 | 1.18 | 0.046 | 3.92% |
| 50 | 1.1646 | 1.1502 | 1.204 | 1.0905 | 1.15 | 0.047 | 4.09% |
| 100 | 1.1285 | 1.04 | 1.1651 | 1.0657 | 1.10 | 0.057 | 5.20% |
| 250 | 0.9685 | 0.9461 | 0.8378 | 0.8835 | 0.91 | 0.060 | 6.55% |
| 500 | 0.6193 | 0.5894 | 0.615 | 0.6246 | 0.61 | 0.016 | 2.55% |

APPENDIX 4.2
Raw Data and Calculated Mean and SD of MTT Assay 12050701
Cell line B16 (10000 cell/well)
Drug treatment (SCV-07 vs 5-Fu)
Drug treatment time 24 hours

| | | | | | | | |
|---|---|---|---|---|---|---|---|
| Conc. (ug/ mL) | OD1 | OD2 | OD3 | OD4 | Mean | SD | CV |

SCV-07

| | | | | | | | |
|---|---|---|---|---|---|---|---|
| 0 | 0.6845 | 0.6673 | 0.6631 | 0.708 | 0.6807 | 0.0204 | 3.00% |
| 0.05 | 0.6626 | 0.6924 | 0.7006 | 0.7459 | 0.7004 | 0.0345 | 4.92% |
| 0.2 | 0.7661 | 0.7629 | 0.7463 | 0.755 | 0.7576 | 0.0088 | 1.17% |
| 0.5 | 0.744 | 0.7374 | 0.7929 | 0.6887 | 0.7408 | 0.0426 | 5.75% |
| 1 | 0.7415 | 0.7574 | 0.7718 | 0.7476 | 0.7546 | 0.0132 | 1.75% |
| 1 | 0.7591 | 0.6618 | 0.7503 | 0.739 | 0.7276 | 0.0446 | 6.13% |
| 2 | 0.7616 | 0.7472 | 0.7613 | 0.7359 | 0.7515 | 0.0124 | 1.65% |
| 5 | 0.7604 | 0.7323 | 0.7637 | 0.7508 | 0.7518 | 0.0141 | 1.88% |
| 10 | 0.7405 | 0.7439 | 0.7174 | 0.6915 | 0.7233 | 0.0243 | 3.35% |
| 20 | 0.7291 | 0.6819 | 0.7529 | 0.7262 | 0.7225 | 0.0296 | 4.10% |
| 50 | 0.5887 | 0.6665 | 0.7022 | 0.677 | 0.6586 | 0.0489 | 7.43% |
| 100 | 0.6107 | 0.5747 | 0.6334 | 0.6176 | 0.6091 | 0.0248 | 4.08% |

5-Fu

| | | | | | | | |
|---|---|---|---|---|---|---|---|
| 0 | 0.6025 | 0.6102 | 0.5967 | 0.5826 | 0.5980 | 0.0117 | 1.95% |
| 0.05 | 0.6949 | 0.6641 | 0.6086 | 0.5543 | 0.6305 | 0.0621 | 9.85% |
| 0.1 | 0.6278 | 0.6051 | 0.6123 | 0.6158 | 0.6153 | 0.0095 | 1.54% |
| 0.2 | 0.5906 | 0.5763 | 0.55 | 0.5112 | 0.5570 | 0.0349 | 6.26% |
| 0.5 | 0.4997 | 0.4868 | 0.4703 | 0.466 | 0.4807 | 0.0155 | 3.23% |
| 1 | 0.4645 | 0.4544 | 0.4483 | 0.4631 | 0.4576 | 0.0076 | 1.67% |
| 2 | 0.4425 | 0.4342 | 0.4424 | 0.4546 | 0.4434 | 0.0084 | 1.90% |
| 5 | 0.4366 | 0.4313 | 0.4241 | 0.4194 | 0.4279 | 0.0076 | 1.78% |
| 10 | 0.4033 | 0.4083 | 0.401 | 0.4063 | 0.4047 | 0.0032 | 0.80% |
| 20 | 0.3805 | 0.3811 | 0.3807 | 0.3761 | 0.3796 | 0.0023 | 0.62% |
| 50 | 0.3363 | 0.3357 | 0.3322 | 0.3286 | 0.3332 | 0.0036 | 1.07% |
| 100 | 0.2875 | 0.2919 | 0.2906 | 0.2729 | 0.2857 | 0.0087 | 3.06% |

APPENDIX 4.3
Raw Data and Calculated Mean and SD of MTT Assay 12050702
Cell line B16 (10000 cell/well)
Drug treatment (SCV-07 vs 5-Fu)
Drug treatment time 24 hours

| | | | | | | | |
|---|---|---|---|---|---|---|---|
| Conc. (ug/mL) | OD1 | OD2 | OD3 | OD4 | Mean | SD | CV |

SCV-07

| | | | | | | | |
|---|---|---|---|---|---|---|---|
| 0 | 0.695 | 0.5837 | 0.6308 | 0.5974 | 0.6267 | 0.0496 | 7.92% |
| 0.05 | 0.6386 | 0.6994 | 0.7424 | 0.7349 | 0.7038 | 0.0474 | 6.73% |
| 0.1 | 0.6938 | 0.7456 | 0.7138 | 0.6847 | 0.7095 | 0.0270 | 3.80% |
| 0.2 | 0.711 | 0.7734 | 0.6973 | 0.6892 | 0.7177 | 0.0382 | 5.32% |
| 0.5 | 0.7112 | 0.7642 | 0.7546 | 0.7006 | 0.7327 | 0.0314 | 4.29% |
| 1 | 0.7452 | 0.7924 | 0.7406 | 0.7118 | 0.7475 | 0.0334 | 4.47% |
| 2 | 0.7075 | 0.7205 | 0.7687 | 0.6784 | 0.7188 | 0.0377 | 5.24% |
| 5 | 0.6359 | 0.7209 | 0.7446 | 0.6954 | 0.6992 | 0.0467 | 6.68% |
| 10 | 0.6511 | 0.7422 | 0.7219 | 0.7425 | 0.7144 | 0.0433 | 6.06% |
| 20 | 0.694 | 0.6401 | 0.6941 | 0.7006 | 0.6822 | 0.0282 | 4.14% |
| 50 | 0.6777 | 0.6159 | 0.5761 | 0.5922 | 0.6155 | 0.0446 | 7.24% |
| 100 | 0.5076 | 0.5969 | 0.5342 | 0.5324 | 0.5428 | 0.0381 | 7.01% |

5-Fu

| | | | | | | | |
|---|---|---|---|---|---|---|---|
| 0 | 0.6183 | 0.6048 | 0.6115 | 0.4979 | 0.5831 | 0.0571 | 9.79% |
| 0.05 | 0.6181 | 0.6008 | 0.5703 | 0.5218 | 0.5778 | 0.0422 | 7.31% |
| 0.1 | 0.6282 | 0.6158 | 0.5688 | 0.5398 | 0.5882 | 0.0412 | 7.00% |
| 0.2 | 0.5831 | 0.5388 | 0.526 | 0.5072 | 0.5388 | 0.0323 | 5.99% |
| 0.5 | 0.4926 | 0.4553 | 0.4541 | 0.4382 | 0.4601 | 0.0231 | 5.01% |
| 1 | 0.4534 | 0.4464 | 0.4319 | 0.4357 | 0.4419 | 0.0098 | 2.23% |
| 2 | 0.4307 | 0.4249 | 0.4251 | 0.3913 | 0.4180 | 0.0180 | 4.31% |
| 5 | 0.4054 | 0.4086 | 0.4028 | 0.3941 | 0.4027 | 0.0062 | 1.54% |
| 10 | 0.3888 | 0.367 | 0.3859 | 0.3523 | 0.3735 | 0.0171 | 4.58% |
| 20 | 0.3756 | 0.3672 | 0.3688 | 0.3629 | 0.3686 | 0.0053 | 1.43% |
| 50 | 0.3154 | 0.3314 | 0.3323 | 0.309 | 0.3220 | 0.0116 | 3.62% |
| 100 | 0.2735 | 0.2699 | 0.2734 | 0.2604 | 0.2693 | 0.0062 | 2.29% |

APPENDIX 4.4
Raw Data and Calculated Mean and SD of MTT Assay 12050703
Cell line B16 (10000 cell/well)
Drug treatment (SCV-07 vs 5-Fu)
Drug treatment time 24 hours

| | | | | | | | |
|---|---|---|---|---|---|---|---|
| Conc. (ug/mL) | OD1 | OD2 | OD3 | OD4 | Mean | SD | CV |

SCV-07

| | | | | | | | |
|---|---|---|---|---|---|---|---|
| 0 | 0.5745 | 0.6092 | 0.5949 | 0.6477 | 0.6066 | 0.0309 | 5.09% |
| 0.05 | 0.6809 | 0.675 | 0.6089 | 0.6452 | 0.6525 | 0.0330 | 5.06% |
| 0.2 | 0.6696 | 0.7292 | 0.6947 | 0.6956 | 0.6973 | 0.0245 | 3.51% |
| 0.5 | 0.7292 | 0.7111 | 0.7482 | 0.7192 | 0.7269 | 0.0160 | 2.20% |
| 1 | 0.6555 | 0.6848 | 0.711 | 0.6885 | 0.6850 | 0.0228 | 3.33% |
| 1 | 0.6734 | 0.7213 | 0.6829 | 0.6622 | 0.6850 | 0.0257 | 3.75% |
| 2 | 0.7587 | 0.7358 | 0.7332 | 0.7322 | 0.7400 | 0.0126 | 1.70% |
| 5 | 0.7289 | 0.7114 | 0.7598 | 0.7487 | 0.7372 | 0.0214 | 2.91% |
| 10 | 0.7394 | 0.7117 | 0.6772 | 0.7079 | 0.7091 | 0.0255 | 3.59% |
| 20 | 0.6577 | 0.6828 | 0.6062 | 0.6144 | 0.6403 | 0.0363 | 5.66% |
| 50 | 0.629 | 0.6333 | 0.6409 | 0.708 | 0.6528 | 0.0371 | 5.69% |
| 100 | 0.5753 | 0.5682 | 0.5656 | 0.6062 | 0.5788 | 0.0187 | 3.23% |

5-Fu

| | | | | | | | |
|---|---|---|---|---|---|---|---|
| 0 | 0.6025 | 0.5566 | 0.5621 | 0.5446 | 0.5665 | 0.0251 | 4.43% |
| 0.05 | 0.6739 | 0.5901 | 0.5928 | 0.5259 | 0.5957 | 0.0606 | 10.18% |
| 0.2 | 0.569 | 0.5109 | 0.5079 | 0.4717 | 0.5149 | 0.0402 | 7.82% |
| 0.5 | 0.4684 | 0.4738 | 0.4973 | 0.4692 | 0.4772 | 0.0136 | 2.86% |
| 1 | 0.4535 | 0.4405 | 0.4448 | 0.435 | 0.4435 | 0.0078 | 1.76% |
| 1 | 0.5876 | 0.5856 | 0.6025 | 0.5704 | 0.5865 | 0.0131 | 2.24% |
| 2 | 0.435 | 0.4246 | 0.4211 | 0.4037 | 0.4211 | 0.0130 | 3.09% |
| 5 | 0.4194 | 0.4031 | 0.4156 | 0.3885 | 0.4067 | 0.0140 | 3.43% |
| 10 | 0.4113 | 0.394 | 0.3915 | 0.3873 | 0.3960 | 0.0106 | 2.66% |
| 20 | 0.3864 | 0.3903 | 0.3819 | 0.3743 | 0.3832 | 0.0069 | 1.79% |
| 50 | 0.3367 | 0.3291 | 0.3243 | 0.3047 | 0.3237 | 0.0137 | 4.22% |
| 100 | 0.2669 | 0.2823 | 0.2876 | 0.272 | 0.2772 | 0.0094 | 3.41% |

APPENDIX 4.5
Raw Data and Calculated Mean and SD of MTT Assay 12060704
Cell line LLC (10000 cells/well)
Drug treatment (SCV-07 vs Cisplatin)
Drug treatment time 48 hours

| | | | | | | | |
|---|---|---|---|---|---|---|---|
| Conc. (ug/mL) | OD1 | OD2 | OD3 | OD4 | Mean | SD | CV |

SCV-07

| | | | | | | | |
|---|---|---|---|---|---|---|---|
| 0 | 1.4131 | 1.3671 | 1.4141 | 1.4641 | 1.4146 | 0.0396 | 2.80% |
| 0.05 | 1.4385 | 1.4471 | 1.3818 | 1.4299 | 1.4243 | 0.0292 | 2.05% |
| 0.1 | 1.4923 | 1.5682 | 1.4111 | 1.3321 | 1.4509 | 0.1019 | 7.03% |
| 0.2 | 1.5528 | 1.5654 | 1.4445 | 1.3831 | 1.4865 | 0.0877 | 5.90% |
| 0.5 | 1.4889 | 1.5723 | 1.4563 | 1.4307 | 1.4871 | 0.0616 | 4.14% |
| 1 | 1.4774 | 1.6092 | 1.4985 | 1.4398 | 1.5062 | 0.0728 | 4.83% |
| 2 | 1.4785 | 1.5705 | 1.4299 | 1.4039 | 1.4707 | 0.0734 | 4.99% |
| 5 | 1.4811 | 1.5539 | 1.4662 | 1.4039 | 1.4763 | 0.0616 | 4.17% |
| 10 | 1.4506 | 1.5419 | 1.4917 | 1.4419 | 1.4815 | 0.0457 | 3.09% |
| 20 | 1.4742 | 1.5821 | 1.4846 | 1.4122 | 1.4883 | 0.0702 | 4.72% |
| 50 | 1.4266 | 1.5641 | 1.6047 | 1.5048 | 1.5251 | 0.0774 | 5.08% |
| 100 | 1.3986 | 1.4666 | 1.4381 | 1.4162 | 1.4299 | 0.0293 | 2.05% |

Cisplatin

| | | | | | | | |
|---|---|---|---|---|---|---|---|
| 0 | 1.4438 | 1.3406 | 1.4 | 1.4602 | 1.4112 | 0.0535 | 3.79% |
| 0.1 | 1.2981 | 1.2868 | 1.4543 | 1.4465 | 1.3714 | 0.0914 | 6.66% |
| 0.2 | 1.2665 | 1.2446 | 1.351 | 1.3193 | 1.2954 | 0.0486 | 3.75% |
| 0.5 | 1.2295 | 1.1064 | 1.2561 | 1.2975 | 1.2224 | 0.0822 | 6.73% |
| 1 | 1.1076 | 1.0239 | 1.0597 | 1.151 | 1.0856 | 0.0555 | 5.11% |
| 2 | 0.9158 | 0.8338 | 0.8627 | 0.8629 | 0.8688 | 0.0342 | 3.93% |
| 5 | 0.6476 | 0.6367 | 0.654 | 0.6724 | 0.6527 | 0.0150 | 2.29% |
| 10 | 0.4029 | 0.4246 | 0.4569 | 0.4347 | 0.4298 | 0.0224 | 5.22% |
| 20 | 0.2586 | 0.2343 | 0.257 | 0.2308 | 0.2452 | 0.0147 | 5.98% |
| 50 | 0.19 | 0.1874 | 0.1826 | 0.1887 | 0.1872 | 0.0032 | 1.73% |
| 100 | 0.1641 | 0.1489 | 0.159 | 0.1593 | 0.1578 | 0.0064 | 4.05% |
| 200 | 0.1241 | 0.1219 | 0.1218 | 0.1242 | 0.1230 | 0.0013 | 1.08% |

APPENDIX 4.6
Raw Data and Calculated Mean and SD of MTT Assay 12060705
Cell line LLC (10000 cells/well)
Drug treatment (SCV-07 vs Cisplatin)
Drug treatment time 48 hours

| | | | | | | | |
|---|---|---|---|---|---|---|---|
| Conc. (ug/mL) | OD1 | OD2 | OD3 | OD4 | Mean | SD | CV |

SCV-07

| | | | | | | | |
|---|---|---|---|---|---|---|---|
| 0 | 1.4272 | 1.3239 | 1.4266 | 1.4842 | 1.4155 | 0.0668 | 4.72% |
| 0.05 | 1.456 | 1.3736 | 1.4003 | 1.4518 | 1.4204 | 0.0402 | 2.83% |
| 0.1 | 1.4119 | 1.3813 | 1.4015 | 1.3273 | 1.3805 | 0.0377 | 2.73% |
| 0.2 | 1.3869 | 1.3807 | 1.4118 | 1.3288 | 1.3771 | 0.0349 | 2.53% |
| 0.5 | 1.3689 | 1.3883 | 1.3435 | 1.312 | 1.3532 | 0.0330 | 2.44% |
| 1 | 1.3774 | 1.4727 | 1.3709 | 1.3566 | 1.3944 | 0.0529 | 3.80% |
| 2 | 1.3888 | 1.3787 | 1.3023 | 1.3347 | 1.3511 | 0.0401 | 2.97% |
| 5 | 1.3882 | 1.4287 | 1.3784 | 1.3639 | 1.3898 | 0.0278 | 2.00% |
| 10 | 1.3838 | 1.3646 | 1.4306 | 1.3676 | 1.3867 | 0.0305 | 2.20% |
| 20 | 1.3617 | 1.4192 | 1.4016 | 1.3575 | 1.3850 | 0.0302 | 2.18% |
| 50 | 1.3541 | 1.4293 | 1.4801 | 1.4914 | 1.4387 | 0.0625 | 4.35% |
| 100 | 1.3945 | 1.3994 | 1.411 | 1.4409 | 1.4115 | 0.0208 | 1.47% |

Cisplatin

| | | | | | | | |
|---|---|---|---|---|---|---|---|
| 0 | 1.339 | 1.3719 | 1.4185 | 1.4699 | 1.3998 | 0.0570 | 4.07% |
| 0.1 | 1.3265 | 1.2827 | 1.4548 | 1.4766 | 1.3852 | 0.0951 | 6.87% |
| 0.2 | 1.1914 | 1.2543 | 1.3664 | 1.3314 | 1.2859 | 0.0785 | 6.10% |
| 0.5 | 1.2099 | 1.072 | 1.2569 | 1.2888 | 1.2069 | 0.0956 | 7.92% |
| 1 | 1.0624 | 0.9971 | 1.0372 | 1.1493 | 1.0615 | 0.0644 | 6.07% |
| 2 | 0.857 | 0.8196 | 0.858 | 0.8878 | 0.8556 | 0.0279 | 3.26% |
| 5 | 0.6211 | 0.6123 | 0.6541 | 0.6823 | 0.6425 | 0.0321 | 4.99% |
| 10 | 0.3884 | 0.4067 | 0.4585 | 0.4381 | 0.4229 | 0.0314 | 7.42% |
| 20 | 0.2526 | 0.2307 | 0.258 | 0.233 | 0.2436 | 0.0137 | 5.64% |
| 50 | 0.186 | 0.1812 | 0.1837 | 0.1882 | 0.1848 | 0.0030 | 1.63% |
| 100 | 0.1609 | 0.1473 | 0.159 | 0.1601 | 0.1568 | 0.0064 | 4.08% |
| 200 | 0.1228 | 0.1226 | 0.1224 | 0.1237 | 0.1229 | 0.0006 | 0.47% |

APPENDIX 4.7
Raw Data and Calculated Mean and SD of MTT Assay 12060706
Cell line LLC (10000 cells/well)
Drug treatment (SCV-07 vs Cisplatin)
Drug treatment time 48 hours

| | | | | | | | |
|---|---|---|---|---|---|---|---|
| Conc. (ug/mL) | OD1 | OD2 | OD3 | OD4 | Mean | SD | CV |

SCV-07

| | | | | | | | |
|---|---|---|---|---|---|---|---|
| 0 | 1.4351 | 1.3309 | 1.4312 | 1.4827 | 1.4200 | 0.0638 | 4.50% |
| 0.05 | 1.4728 | 1.3554 | 1.4340 | 1.4665 | 1.4322 | 0.0539 | 3.77% |
| 0.1 | 1.4237 | 1.3684 | 1.3839 | 1.3602 | 1.3841 | 0.0282 | 2.04% |
| 0.2 | 1.3918 | 1.3477 | 1.4257 | 1.3061 | 1.3678 | 0.0521 | 3.81% |
| 0.5 | 1.3643 | 1.3710 | 1.3840 | 1.2949 | 1.3536 | 0.0399 | 2.95% |
| 1 | 1.3714 | 1.4547 | 1.4033 | 1.3810 | 1.4026 | 0.0372 | 2.65% |
| 2 | 1.3762 | 1.3586 | 1.3582 | 1.3631 | 1.3640 | 0.0084 | 0.62% |
| 5 | 1.3890 | 1.3812 | 1.3867 | 1.3997 | 1.3892 | 0.0078 | 0.56% |
| 10 | 1.3536 | 1.3438 | 1.4203 | 1.3918 | 1.3774 | 0.0353 | 2.56% |
| 20 | 1.3870 | 1.3834 | 1.4179 | 1.3917 | 1.3950 | 0.0156 | 1.12% |
| 50 | 1.3991 | 1.4275 | 1.4750 | 1.5036 | 1.4513 | 0.0469 | 3.23% |
| 100 | 1.4015 | 1.3835 | 1.4165 | 1.4369 | 1.4096 | 0.0227 | 1.61% |

Cisplatin

| | | | | | | | |
|---|---|---|---|---|---|---|---|
| 0 | 1.3262 | 1.3668 | 1.4249 | 1.4716 | 1.3974 | 0.0639 | 4.58% |
| 0.1 | 1.3274 | 1.3206 | 1.4551 | 1.4797 | 1.3957 | 0.0834 | 5.98% |
| 0.2 | 1.2235 | 1.2814 | 1.3661 | 1.3362 | 1.3018 | 0.0629 | 4.83% |
| 0.5 | 1.2338 | 1.1176 | 1.2645 | 1.2875 | 1.2259 | 0.0754 | 6.15% |
| 1 | 1.0775 | 1.0392 | 1.0629 | 1.1371 | 1.0792 | 0.0417 | 3.87% |
| 2 | 0.8508 | 0.8616 | 0.8627 | 0.8901 | 0.8663 | 0.0168 | 1.93% |
| 5 | 0.6228 | 0.6287 | 0.6673 | 0.6841 | 0.6507 | 0.0297 | 4.57% |
| 10 | 0.3873 | 0.4067 | 0.4589 | 0.4376 | 0.4226 | 0.0318 | 7.53% |
| 20 | 0.2473 | 0.2358 | 0.2583 | 0.2328 | 0.2436 | 0.0117 | 4.78% |
| 50 | 0.1833 | 0.1825 | 0.1841 | 0.1885 | 0.1846 | 0.0027 | 1.45% |
| 100 | 0.1612 | 0.1493 | 0.1591 | 0.1599 | 0.1574 | 0.0055 | 3.46% |
| 200 | 0.1238 | 0.1223 | 0.1222 | 0.1239 | 0.1231 | 0.0009 | 0.75% |

APPENDIX 4.8
Raw Data and Calculated Mean and SD of MTT Assay 12070704
Cell line RenCa (7000 cells/well)
Drug treatment (SCV-07 vs 5-Fu)
Drug treatment time 72 hours

| | | | | | | | |
|---|---|---|---|---|---|---|---|
| Conc. (ug/mL) | OD1 | OD2 | OD3 | OD4 | Mean | SD | CV |

SCV-07

| | | | | | | | |
|---|---|---|---|---|---|---|---|
| 0 | 1.1512 | 1.0942 | 1.1050 | 1.0829 | 1.1083 | 0.0300 | 2.70% |
| 0.05 | 1.0374 | 1.0135 | 1.0011 | 0.9291 | 0.9953 | 0.0466 | 4.68% |
| 0.1 | 1.0565 | 1.0203 | 1.0421 | 1.0147 | 1.0334 | 0.0194 | 1.88% |
| 0.2 | 1.0777 | 1.0217 | 0.9710 | 1.0184 | 1.0222 | 0.0437 | 4.27% |
| 0.5 | 1.0957 | 1.0963 | 1.0017 | 1.0202 | 1.0535 | 0.0497 | 4.72% |
| 1 | 1.0492 | 1.0211 | 0.9666 | 1.0728 | 1.0274 | 0.0457 | 4.45% |
| 2 | 1.0209 | 1.0683 | 1.0650 | 1.0673 | 1.0554 | 0.0230 | 2.18% |
| 5 | 1.0664 | 1.1032 | 0.9598 | 1.0856 | 1.0538 | 0.0644 | 6.11% |
| 10 | 0.9690 | 1.0370 | 1.0541 | 1.0158 | 1.0190 | 0.0368 | 3.61% |
| 20 | 1.1018 | 1.1023 | 0.9306 | 1.0288 | 1.0409 | 0.0812 | 7.80% |
| 50 | 1.0275 | 1.0561 | 0.9939 | 0.9547 | 1.0081 | 0.0437 | 4.34% |
| 100 | 0.9803 | 0.9369 | 1.0102 | 0.9290 | 0.9641 | 0.0381 | 3.95% |

5-Fu

| | | | | | | | |
|---|---|---|---|---|---|---|---|
| 0 | 1.0886 | 1.0952 | 1.0514 | 1.0915 | 1.0817 | 0.0204 | 1.88% |
| 0.05 | 0.6485 | 0.6341 | 0.6411 | 0.6063 | 0.6325 | 0.0184 | 2.91% |
| 0.1 | 0.4897 | 0.4751 | 0.5134 | 0.5262 | 0.5011 | 0.0230 | 4.59% |
| 0.2 | 0.4176 | 0.3867 | 0.3894 | 0.3800 | 0.3934 | 0.0166 | 4.22% |
| 0.5 | 0.3363 | 0.3221 | 0.3346 | 0.3203 | 0.3283 | 0.0083 | 2.52% |
| 1 | 0.3088 | 0.2917 | 0.2998 | 0.3067 | 0.3018 | 0.0077 | 2.56% |
| 2 | 0.2875 | 0.2766 | 0.2880 | 0.3072 | 0.2898 | 0.0127 | 4.39% |
| 5 | 0.3027 | 0.2948 | 0.2819 | 0.2933 | 0.2932 | 0.0086 | 2.92% |
| 10 | 0.2745 | 0.2829 | 0.2743 | 0.2925 | 0.2811 | 0.0086 | 3.07% |
| 20 | 0.2639 | 0.2577 | 0.2471 | 0.2730 | 0.2604 | 0.0109 | 4.18% |
| 50 | 0.2221 | 0.2255 | 0.2151 | 0.2263 | 0.2223 | 0.0051 | 2.30% |
| 100 | 0.2036 | 0.1959 | 0.1882 | 0.2070 | 0.1987 | 0.0084 | 4.22% |

APPENDIX 4.9
Raw Data and Calculated Mean and SD of MTT Assay 12070705
Cell line RenCa (7000 cells/well)
Drug treatment (SCV-07 vs 5-Fu)
Drug treatment time 72 hours

| | | | | | | | |
|---|---|---|---|---|---|---|---|
| Conc. (ug/mL) | OD1 | OD2 | OD3 | OD4 | Mean | SD | CV |

SCV-07

| | | | | | | | |
|---|---|---|---|---|---|---|---|
| 0 | 1.1302 | 1.0936 | 1.1067 | 1.0937 | 1.1061 | 0.0172 | 1.56% |
| 0.05 | 1.0943 | 1.0399 | 0.9717 | 1.0023 | 1.0271 | 0.0528 | 5.14% |
| 0.1 | 1.0329 | 1.0464 | 0.9478 | 1.0264 | 1.0134 | 0.0445 | 4.39% |
| 0.2 | 1.0809 | 1.0599 | 1.0848 | 0.9881 | 1.0534 | 0.0449 | 4.26% |
| 0.5 | 1.0460 | 1.0517 | 0.9973 | 1.0159 | 1.0277 | 0.0257 | 2.50% |
| 1 | 1.0780 | 1.1082 | 1.0618 | 0.9667 | 1.0537 | 0.0611 | 5.80% |
| 2 | 1.0990 | 1.1076 | 1.1703 | 1.0328 | 1.1024 | 0.0563 | 5.10% |
| 5 | 1.0603 | 1.0883 | 1.1130 | 1.0614 | 1.0808 | 0.0251 | 2.32% |
| 10 | 1.0470 | 1.1484 | 1.1144 | 1.0625 | 1.0931 | 0.0468 | 4.28% |
| 20 | 1.1738 | 1.1348 | 1.0721 | 1.0460 | 1.1067 | 0.0582 | 5.26% |
| 50 | 1.0781 | 1.1069 | 1.0368 | 1.0795 | 1.0753 | 0.0289 | 2.69% |
| 100 | 1.0145 | 1.0008 | 1.0017 | 0.9641 | 0.9953 | 0.0217 | 2.18% |

5-Fu

| | | | | | | | |
|---|---|---|---|---|---|---|---|
| 0 | 1.1518 | 1.1850 | 1.1659 | 1.2038 | 1.1766 | 0.0227 | 1.93% |
| 0.05 | 0.6016 | 0.6238 | 0.5999 | 0.6511 | 0.6191 | 0.0240 | 3.87% |
| 0.1 | 0.4858 | 0.4996 | 0.5300 | 0.5037 | 0.5048 | 0.0185 | 3.66% |
| 0.2 | 0.3779 | 0.4014 | 0.3843 | 0.4218 | 0.3964 | 0.0197 | 4.96% |
| 0.5 | 0.3096 | 0.2969 | 0.3027 | 0.3181 | 0.3068 | 0.0091 | 2.98% |
| 1 | 0.3073 | 0.2830 | 0.2847 | 0.2981 | 0.2933 | 0.0115 | 3.93% |
| 2 | 0.2980 | 0.2903 | 0.2744 | 0.2933 | 0.2890 | 0.0102 | 3.54% |
| 5 | 0.3027 | 0.2859 | 0.2876 | 0.2945 | 0.2927 | 0.0076 | 2.61% |
| 10 | 0.2929 | 0.2816 | 0.2719 | 0.2777 | 0.2810 | 0.0089 | 3.15% |
| 20 | 0.2585 | 0.2576 | 0.2555 | 0.2560 | 0.2569 | 0.0014 | 0.54% |
| 50 | 0.2216 | 0.2162 | 0.2188 | 0.2141 | 0.2177 | 0.0032 | 1.49% |
| 100 | 0.1911 | 0.1943 | 0.1915 | 0.2014 | 0.1946 | 0.0048 | 2.45% |

APPENDIX 4.10
Raw Data and Calculated Mean and SD of MTT Assay 12070706
Cell line RenCa (7000 cells/well)
Drug treatment (SCV-07 vs 5-Fu)
Drug treatment time 72 hours

| | | | | | | | |
|---|---|---|---|---|---|---|---|
| Conc. (ug/mL) | OD1 | OD2 | OD3 | OD4 | Mean | SD | CV |

SCV-07

| | | | | | | | |
|---|---|---|---|---|---|---|---|
| 0 | 1.1688 | 1.1034 | 1.1105 | 1.1218 | 1.1261 | 0.0294 | 2.61% |
| 0.05 | 1.0531 | 1.0261 | 0.9896 | 1.0755 | 1.0361 | 0.0370 | 3.57% |
| 0.1 | 1.0397 | 1.0312 | 1.0266 | 1.0184 | 1.0290 | 0.0089 | 0.86% |
| 0.2 | 1.0400 | 1.0216 | 0.9879 | 1.0103 | 1.0150 | 0.0218 | 2.15% |
| 0.5 | 1.0939 | 1.0756 | 0.9939 | 1.0552 | 1.0547 | 0.0435 | 4.12% |
| 1 | 1.0625 | 1.0415 | 0.9779 | 1.0328 | 1.0287 | 0.0361 | 3.51% |
| 2 | 1.0392 | 1.0737 | 1.0311 | 1.0346 | 1.0447 | 0.0196 | 1.88% |
| 5 | 1.0714 | 1.0735 | 0.9572 | 1.0544 | 1.0391 | 0.0553 | 5.32% |
| 10 | 0.9808 | 1.0795 | 1.0532 | 1.0185 | 1.0330 | 0.0428 | 4.15% |
| 20 | 1.0890 | 1.0875 | 0.9739 | 1.0470 | 1.0494 | 0.0539 | 5.14% |
| 50 | 1.0359 | 1.0173 | 0.9625 | 0.9875 | 1.0008 | 0.0324 | 3.24% |
| 100 | 0.9768 | 0.9460 | 1.0235 | 0.9533 | 0.9749 | 0.0350 | 3.59% |

5-Fu

| | | | | | | | |
|---|---|---|---|---|---|---|---|
| 0 | 1.1193 | 1.1103 | 1.0773 | 1.1065 | 1.1034 | 0.0182 | 1.65% |
| 0.05 | 0.6672 | 0.6226 | 0.6392 | 0.6112 | 0.6351 | 0.0243 | 3.83% |
| 0.1 | 0.4884 | 0.4797 | 0.5191 | 0.5259 | 0.5033 | 0.0227 | 4.50% |
| 0.2 | 0.4135 | 0.3988 | 0.4097 | 0.3818 | 0.4010 | 0.0142 | 3.54% |
| 0.5 | 0.3272 | 0.3209 | 0.3339 | 0.3217 | 0.3259 | 0.0060 | 1.84% |
| 1 | 0.3059 | 0.3016 | 0.2980 | 0.3086 | 0.3035 | 0.0047 | 1.54% |
| 2 | 0.2928 | 0.2838 | 0.2900 | 0.3075 | 0.2935 | 0.0100 | 3.42% |
| 5 | 0.2995 | 0.2954 | 0.2865 | 0.2940 | 0.2939 | 0.0054 | 1.85% |
| 10 | 0.2794 | 0.2851 | 0.2808 | 0.2921 | 0.2844 | 0.0057 | 2.01% |
| 20 | 0.2652 | 0.2630 | 0.2465 | 0.2710 | 0.2614 | 0.0105 | 4.02% |
| 50 | 0.2299 | 0.2252 | 0.2186 | 0.2268 | 0.2251 | 0.0048 | 2.12% |
| 100 | 0.2079 | 0.1980 | 0.1936 | 0.2078 | 0.2018 | 0.0072 | 3.56% |

1. A method of treatment for treating, at least partly preventing, inhibiting or reducing lung cancer, a metastasis thereof, or a metastasis in a lung from a cancer outside the lung, or for treating, at least partly preventing, inhibiting or reducing growth of lung cancer cells, a metastasis thereof, or a metastasis of cancer cells in a lung from cancer cells outside the lung, in a subject, comprising administering to the subject an effective amount of an immunomodulator compound of formula A 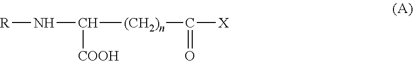
wherein, n is 1 or 2, R is hydrogen, acyl, alkyl or a peptide fragment, and X is an aromatic or heterocyclic amino acid or a derivative thereof, so as to treat, at least partly prevent, inhibit or reduce said lung cancer, said metastasis thereof, or said metastasis in a lung from a cancer outside the lung in the subject, or so as to treat, at least partly prevent, inhibit or reduce growth of said lung cancer cells, said metastasis thereof, or said metastasis of cancer cells in said lung from cancer cells outside the lung, in the subject.

2. The method of claim 1, wherein X is L-tryptophan or D-tryptophan.

3. The method of claim 1 wherein said compound is SCV-07.

4. The method of claim 1 wherein said compound is administered at a dosage within a range of about 0.001-1000 mg.

5. The method of claim 1 wherein said compound is administered at a dosage within a range of about 0.01-100 mg.

6. The method of claim 1 wherein said compound is administered at a dosage within a range of about 0.00001-1000 mg/kg subject body weight.

7. The method of claim 1 wherein said compound is administered at a dosage within a range of about 0.01-100 mg/kg subject body weight.

8. The method of claim 1 wherein said compound is SCV-07, and is administered at a dosage within a range of about 0.001-1000 mg.

9. The method of claim 1 wherein said compound is SCV-07, and administered at a dosage within a range of about 0.00001-1000 mg/kg subject body weight.

10. The method of claim 3 wherein said treatment is for primary lung cancer.

11. The method of claim 10 wherein said compound is administered at a dosage within a range of about 0.001-1000 mg.

12. The method of claim 10 wherein said compound is administered at a dosage within a range of about 0.1-100 mg.

13. The method of claim 10 wherein said compound is administered at a dosage within a range of about 0.00001-1000 mg/kg subject body weight.

14. The method of claim 10 wherein said compound is administered at a dosage within a range of about 0.01-100 mg/kg subject body weight.

15. The method of claim 3 wherein said treatment is for a lung cancer metastasis.

16. The method of claim 15 wherein said compound is administered at a dosage within a range of about 0.001-1000 mg.

17. The method of claim 15 wherein said compound is administered at a dosage within a range of about 0.1-100 mg.

18. The method of claim 15 wherein said compound is administered at a dosage within a range of about 0.00001-1000 mg/kg subject body weight.

19. The method of claim 15 wherein said compound is administered at a dosage within a range of about 0.01-100 mg/kg subject body weight.

20. The method of claim 12 wherein said dosage is about 10 mg.

21. The method of claim 17 wherein said dosage is about 10 mg.

22. The method of claim 1 wherein said compound of formula A is administered in a treatment regimen additionally including administration of at least one of radiation or a chemotherapy agent.

23. The method of claim 22 wherein said chemotherapy agent comprises at least one of cisplatin, 5-Fu or DTIC.
